(19) United States
(12) Patent Application Publication
(10) Pub. No.: US 2025/0041346 A1
(43) Pub. Date: Feb. 6, 2025

(54) THYMIC EPITHELIAL STEM CELLS

(71) Applicant: THE FRANCIS CRICK INSTITUTE LIMITED, London (GB)

(72) Inventors: Paola Bonfanti, London (GB); Roberta Ragazzini, London (GB)

(21) Appl. No.: 18/847,886

(22) PCT Filed: Mar. 17, 2023
(86) PCT No.: PCT/EP2023/056975

§ 371 Date: Sep. 17, 2024

(30) Foreign Application Priority Data
Mar. 17, 2022 (GB) 2203756.8

(51) Int. Cl. A61K 35/26; C12N 5/078

(52) U.S. Cl. CPC A61K 35/26; C12N 5/065

(57) ABSTRACT

The present invention relates to a newly identified population of thymic epithelial stem cells, methods for their isolation, culture and differentiation, and uses of the cells, in particular their use in therapy, creation of therapeutic thymic constructs and drug screening.

### FIELD OF THE INVENTION

The present invention relates to a newly identified population of thymic epithelial stem cells, methods for their isolation, culture and differentiation, and uses of the cells, in particular their use in therapy, creation of therapeutic thymic constructs and drug screening.

### BACKGROUND

Epithelia are the paradigm of tissues that constantly renew during development, homeostasis, and regeneration. These processes are driven by self-renewing epithelial stem cells (SC), the only bona fide stem cells that can be extensively expanded in vitro [1,2,3,4,5,6,7].

Stemness maintenance largely relies on the crosstalk of SC with their specialized in vivo microenvironment, a.k.a. niche. Co-culture with irradiated feeder fibroblasts for stratified epithelial SC or Paneth cells for intestinal SC, provide evidence of the importance of the niche signals to expand functional SC for several generations in vitro [8,9]. With rare exceptions (i.e. LGR5 in the gut crypt epithelium), most of the ‘professional’ epithelial SC in the human body do not present unique markers and are defined by multiple phenotypic and functional features [10,11]. Nonetheless, high expression of the TP63 transcription factor (TF)—especially of its ΔNTP63α isoform—has been correlated with epithelial stemness in culture [12,13,14]. More recently, a transcriptional signature has been reported for the epidermal ‘holoclone’—the keratinocyte clonogenic SC capable of self-renewal in vitro and in vivo [15].

The thymus stands out among organs for the unique three-dimensional (3D) morphological complexity of its epithelium, and for undergoing progressive atrophy during postnatal life [16]. The thymus is the primary lymphoid organ essential for T cell development and is uniquely necessary for the generation and selection of a diverse yet self-tolerant T cell repertoire during foetal development and early postnatal life. This reflects critical spatial and temporal interactions of developing thymocytes with the thymic stroma, which is composed of different types of thymic epithelial cells (medullary (m)TEC and cortical (c)TEC), myoid (MC) and neuroendocrine cells (NEC), thymic interstitial cells (TIC), endothelial cells (EC), and haematopoietic subtypes such as dendritic cells (DCs), B cells and macrophages (M0). Haematopoietic cells colonise the epithelial-interstitial thymic anlagen during development and promote lympho-stroma crosstalk that orchestrates both thymocyte development and epithelial differentiation and morphogenesis. Failure of thymic epithelial specification during development results in congenital thymic agenesis and leads to severe immunodeficiency and autoimmunity [17,18]. Embryonic thymic epithelial progenitors and their differentiation into cTEC and mTEC have been extensively studied, e.g. using lineage tracing, reporter systems or transplantation in mouse models [19,20].

In contrast, postnatal epithelial stem/progenitor cells able to maintain thymus homeostasis and the mechanisms contributing to thymus repair and regeneration are still poorly understood [20,21,22,23,44]. This gap in understanding has led to a paucity of therapeutic strategies to address diseases and disorders associated with thymus. Greater insight into the cellular origins of thymic tissue has the potential to reveal new therapeutic strategies applicable to a broad range of medical conditions, including primary and acquired immunodeficiencies, autoimmune diseases and thymic atrophy or athymia.

To address this gap in understanding, the inventors have performed in vivo and in vitro single cell analysis at high resolution combined with prospective isolation and differentiation assays, that have allowed the identification and characterisation of stem cells of the postnatal human thymus. The inventors have further identified methods for the isolation and culture of these thymic epithelial stem cells (TESCs), as well as methods for downstream differentiation of TESCs into heterogenous thymic epithelial cell types.

### SUMMARY OF THE INVENTION

In a first aspect, the invention provides an isolated thymic epithelial stem cell, wherein the isolated thymic epithelial stem cell is BCAM^{pos}, CD49F^{pos}, CD90^{pos} and CD24^{neg}.

In a second aspect, the invention provides an isolated thymic epithelial stem cell, wherein the isolated thymic epithelial stem cell is CD49F^{pos} and CD90^{pos}.

In a third aspect, the invention provides an isolated thymic epithelial stem cell, wherein the isolated thymic epithelial stem cell is BCAM^{pos}.

In a fourth aspect, the invention provides an isolated thymic epithelial stem cell, wherein the isolated thymic epithelial stem cell is capable of ex vivo self-renewal.

In a fifth aspect, the invention provides an isolated thymic epithelial stem cell, wherein the isolated thymic epithelial stem cell exhibits long-term expansion capacity in vitro.

In a sixth aspect, the invention provides an isolated thymic epithelial stem cell, wherein the isolated thymic epithelial stem cell is capable of differentiating into cortical thymic epithelial cells and/or medullary epithelial cells.

In a seventh aspect, the invention provides a method for isolating a thymic epithelial stem cell from thymus, the method comprising:
(a) obtaining a thymic tissue sample,
(b) isolating thymic epithelial cells from the thymic tissue sample to obtain a thymic epithelial cell fraction, and
(c) isolating BCAM^{pos}CD49F^{pos}CD90^{pos}CD24^{neg} thymic epithelial cells from the thymic epithelial cell fraction to obtain isolated thymic epithelial stem cells.

In an eighth aspect, the invention provides a method of culturing a thymic epithelial stem cell comprising:
(a) providing at least one isolated thymic epithelial stem cell, and
(b) culturing the at least one isolated thymic epithelial stem cell under conditions suitable for maintenance and expansion of the at least one isolated thymic epithelial stem cell.

In a ninth aspect, the invention provides a method of culturing a cortical thymic epithelial cell derived from a thymic epithelial stem cell, comprising:
(a) providing a thymic epithelial stem cell; and
(b) culturing the thymic epithelial stem cell under conditions suitable for obtaining cortical thymic epithelial cells.

In a tenth aspect, the invention provides a method of differentiating an isolated thymic epithelial stem cell, comprising:
(a) providing at least one isolated thymic epithelial stem cell,
(b) contacting the at least one isolated thymic epithelial stem cell to a membrane, wherein the at least one isolated thymic epithelial stem cell is in contact with an upper surface of the membrane,
(c) providing a cell culture medium, wherein the cell culture medium is positioned below a lower surface of the membrane.

In an eleventh aspect, the invention provides a thymic construct suitable for implantation into a subject comprising an isolated thymic epithelial stem cell as described herein.

In a twelfth aspect, the invention provides a method of treating a disease or disorder in a subject, comprising administering to the subject an isolated thymic epithelial stem cell or a thymic construct as described herein.

In a thirteenth aspect, the invention provides a method of producing a thymic construct suitable for implantation into a subject, the method comprising the steps of:
(a) providing an acellular scaffold;
(b) seeding the acellular scaffold with an isolated thymic epithelial stem cell as described herein; and
(c) culturing the seeded scaffold to produce said construct.

In some embodiments, the isolated thymic epithelial stem cell of the invention is capable of ex vivo self-renewal.

In some embodiments, the isolated thymic epithelial stem cell of the invention exhibits long-term expansion capacity in vitro.

### BRIEF DESCRIPTION OF THE DRAWINGS

FIG. 1—Human postnatal thymic epithelial cells (TEC) are highly heterogenous and contain a Polykeratin cluster. (A) UMAP plot visualisation of thymic epithelial cells coloured by cell cluster group. (B) UMAP visualisation of log 10 expression of cytokeratin genes (KRT13, KRT14, KRT15, KRT17) expressed in the Polykeratin cluster (C) Average expression of Polykeratin cluster markers identified by linear regression in orange against expression in all the other cells. (D) UMAP category feature view plot of Polykeratin cluster marker genes log 10 expression for the following: CEBPD, CLU, FN1, IFITM3, TIMP1, VCAM1, CTFG, TAGLN and CH25H. (E) UMAP category feature view plots and dot plots showing gene category cortical TEC, (F) mTEC Myo, and (H) and mTEC Neuroendocrine; the genes list defining each of these categories are indicated on the left side of the plots. (G) UMAP plot showing holoclone signature expressed by polykeratin cells (thymic epithelial stem cells); (I) Category enrichment analysis of DEGs of Polykeratin cluster versus all the other clusters. Each pathway is specified on the Y axis; −log(FDR) is represented in the X axis. Color code illustrates ten mostly upregulated categories in Polykeratin cluster (orange) and downregulated ones in gray. Hypergeometric test was performed on the top up- and downregulated genes to identify overrepresented gene categories.

FIG. 2—Single-cell trajectory analysis reveals that mTEC and cTEC compartments differentiate from Polykeratin cells. (A) UMAP plot of cells coloured according to Pseudotime analysis subtrajectories run with Monocle software: Polykeratin cells differentiate along trajectory towards mTEC-Myo (left panel) and mTEC-Neuro (middle panel); Polykeratin differentiate towards cortical clusters (right panel), cells. (B, C) Pseudotime Heatmap depicting the most time-variable genes along the single-cell trajectory from Polykeratin to mTEC-differentiating and mTEC-Neuro (B) and mTEC-Myo (C). Cluster colours and Pseudotime are indicated at the top of X axes and variable genes categories are listed on the Y axes. (D) Ouija Pseudotime heatmap indicating trajectory from Polykeratin to cTEC clusters. Highlighted genes along the trajectory as well as category of genes are indicated on the left and right side of the graph respectively. Clusters, pseudotime value and log 10 expression are indicated in the legends on the right. Cluster colors and Pseudotime are indicated at the top of x axes. (E) Single gene log 10 expression plots are showed for relevant genes categories, expressed along Ouija Pseudotime trajectory: Polykeratin (CLU), cTEC differentiating (ATF3, CCL5) and mature cTEC (CTSV, PRSS16, PSMB111).

FIG. 3—Prospective isolation of mTEC and cTEC Polykeratin stem cells. (A) Representative FACS analysis of dissociated and enriched thymic cells for N=5 human postnatal thymic (3 days to 5 years old donors). cTEC and mTEC cells were gated for CD49F^{pos} expression and further subdivided and sorted according to CD24 expression. A total of four mTEC populations were isolated: CD49F^{pos}CD90^{pos}CD24^{neg}, CD49F^{pos}CD90^{neg}CD24^{pos}, CD49F^{neg}CD90^{pos}CD24^{neg}, CD49F^{neg}CD90^{neg}CD24^{pos}; and two cTEC populations: CD49F^{pos}CD90^{pos}CD24^{neg} and CD49F^{neg}CD90^{int}CD24^{neg}. (B) Mean fluorescence intensity quantification of CD90 in CD24^{pos} and CD24neg cells. (C, D) Rhodamine-B staining of each mTEC and cTEC sorted population after two passages in culture: 500 cells were seeded in a dish for colony-forming efficiency (CFE) assay; the dish was fixed and stained with Rhodamine-B after 12 days of culture. Cells gave rise to colonies of variable sizes that stained either strongly or deem with Rhodamine-B. Different Number of colonies indicated diverse clonogenic potential of each population (N=4).

FIG. 4—Single-cell analysis demonstrates heterogeneity of TEC in vitro and defines a thymic-specific cell signature. (A) UMAP plot visualization of cultivated epithelial cells coloured by cell cluster group per each representative sample. (B) Thymic Polykeratin stem cell markers were expressed by Cluster 1 present only in thymic cultures: average expression of Cluster 1 genes is plotted against average expression in all the other clusters and upregulated genes are displayed in pink. (C) UMAP plot visualisation (log 10 expression) of marker genes across clusters indicate heterogenous transcriptional profiles in cultivated TEC upregulating hybrid epithelial-mesenchymal features (THY1, C1), stratification (C2) and cornification/terminal differentiation (C3) markers. (D, upper panel) Representative FACS sorting plots of cultivated thymic epithelial cells (N=5). Feederneg/CD49F^{pos} thymic epithelial cells were sorted for EpCAM^{neg}, EpCAM^{pos}CD24^{neg} and EpCAM^{pos}CD24^{pos} (mid and right panels). (D, lower panel) Rhodamine-B staining of EpCAM^{neg}, EpCAM^{pos}CD24^{neg} and EpCAM^{pos}CD24^{pos} sorted populations (N=3). 500 sorted cells were plated in a dish for colony-forming efficiency (CFE); the dish was stained after 12 days of culture. Cells gave rise to colonies of variable size that stained differently with Rhodamine-B.

FIG. 5—Refractive-edges TEC represent thymic stem cells expanding in vitro. (A) Schematic visualising single-cell cloning of cultured TEC. (B) Phase contrast images of individual TEC colonies. TEC were classified according to their cell morphology and colony pattern as Refractive-edges, Scattered/Refractive-edges and Stratified. Keratinocytes are classified only as Stratified colonies. (C) Immunofluorescence labelling against epithelial and mesenchymal markers on expanding thymic refractive-edges (left top panels), scattered (right top panels), stratified (left bottom panels) and keratinocytes (right bottom panels) colonies. Most of TEC in culture co-expressed cytokeratin (KRT)5/14 (yellow) and KRT8 (cyan). While TE-7 (Magenta) was expressed by refractive-edges and scattered colonies, but not by stratified and keratinocyte colonies (each first row). In contrast, E-Cadherin/CDH1 (Magenta) and EpCAM (yellow) were highly expressed by cells in stratified colonies and keratinocytes. CD49F (cyan) was clearly detected in all cells. Nuclei counterstained with DAPI. N=4, Scale bar, 50 μm. (D) Immunofluorescence staining for CD90 (THY1), IFITM3 and FN1 on expanding thymic epithelial (KRT5/14) colonies. IFITM3 or FN1 (Magenta) and CD90 (THY1, Cyan) were expressed by refractive-edges and scattered colonies, but not by stratified or keratinocyte colonies; FN1 was detected also in mouse feeder cells. N=4, Scale bar, 50 μm.

FIG. 6—In vitro expanded Polykeratin stem cells differentiate into both cortical and medullary fates. (A) Immunofluorescence staining of differentiated cTEC and mTEC Polykeratin cells showed differentiation potency toward both cortex (CD205, green) and medulla (KRT5/14, red); N=5. (B) Immunostaining of cells upon differentiation (top panels: left low magnification and right high magnification) and human postnatal thymus 7 mM section (bottom panels, left low magnification and right high magnification) showing in red KRT5-14 and in gray KRT10. White arrows indicate KRT7 positive cells in green. (N=5 assays and N=3 Human Postnatal thymi). (C) RT-qPCR analysis of cultivated TEC in 2D expansion (black dots) versus TEC after differentiation: cortical genes (CD205, KNCIP3, CD74 and CSTV) in green and medullary genes (CLDN4, MYOG, SOX2 and SYP) in red. Relative gene expression to HPRT housekeeping is shown in Y axis; significance: Mann-Whitney test, non-parametric; *p<0.05; **p<0.01, N≥4, mean±S.E.M.

FIG. 7—Schematic workflow for thymic SC differentiation assay from a single clone, created with Biorender.com. Differentiated clones show cells positive for KRT5 (red), cortical cells (LY75-positive, green) on the left and areas with Hassall's Bodies (HB) structures positive for KRT10 (cyan) on the right. Nuclei counterstained with DAPI. Scale bar, 50 μm (representative image, N=5, independent clones).

FIG. 8—Immunofluorescent images of thymic scaffold grafts (16 weeks post transplantation, wpt) sections (7 μm) stained for cortical and HB region: left panels LY75 in green for cortical cells, KRT5 (red) and KRT10 (gray) for medullary cells. Right panels Immunofluorescence staining of 16wpt grafts sections: ASCL1 (magenta) and KRT18 (gray) for medullary transition cells and KRT18 (gray) SOX2 (cyan) for neuroendocrine cells. Nuclei are counterstained with DAPI. Scale bar, 50 μm (N=8, repopulated scaffolds per time point).

FIG. 9—RT-qPCR analysis of expanded thymic cells in 2D expansion (black dots) versus differentiation showing upregulation of cortical genes (LY75, KCNIP3, CD74, CSTV, CD274, FOXN1) in green (top panel) and medullary genes (CLDN4, MYOG, SOX2, SYP, ASCL1, CLDN3, SOX11) in red (bottom panel). RT-qPCR analysis of thymic clones in 2D expansion (black dots) versus thymic clones after differentiation indicated upregulation of cortical genes (LY75, KCNIP3) in green dots and medullary genes (CLDN4, MYOG, SOX2) in red squares. Relative gene expression to HPRT housekeeping is shown in y axis; significance: Mann-Whitney test, non-parametric; *p<0.05; **p<0.01, N=5, independent clones; mean±S.E.M.

FIG. 10—Polykeratin cells reside within subcapsular and perivascular niches in vivo. (A) Low and High magnification images showing immunofluorescence labelling of thymic epithelial cells in human thymi co-stained with anti-ITGA6 (CD49f) antibody (yellow), FN1 (magenta) and EpCAM (cyan). Left panels show co-staining in the subcapsular region at low and high magnification; right panels show co-staining in medullary area. Arrows (white) highlight individual triple positive cells. (N=4, human thymi). (B) Immunofluorescence labelling of human post-natal thymic sections by co-staining of anti-KRT15 antibody (yellow), KRT13 (magenta) or KRT8-18 (cyan) at high magnification. Left panel shows co-staining in subcapsular region and right panel in medulla. Arrows (white) highlight individual cells with triple co-staining (N=4).

FIG. 11—Prospective isolation of clonogenic mTEC and cTEC. (A) Volcano plot analysis of freshly sorted clonogenic versus nonclonogenic thymic epithelial cells. All genes present on the Stem Cell NanoString nCounter Panel-Plus have been plotted. Each dot represents one gene. The log fold-change value of the sorted BCAM^{pos} versus BCAM^{neg} cells is represented on the x-axis. The y-axis shows the log 10 of the adjusted p-value. A p-value of 0.05 and a fold change of 2 are indicated by gray highlighting the most significantly upregulated (red) and downregulated (blue) genes (N=3, human thymic samples). (B) Single gene expression profiles of adhesion molecules and surface markers expressed by BCAM^{pos} and BCAM^{neg} TEC via nCounter NanoString Technologies (N=3, human thymic samples).

### DETAILED DESCRIPTION OF THE INVENTION

The thymus is critical for T cell development and establishment of the vast T cell repertoire necessary for effective immune function. Thymopoiesis (the process of T cell development within the thymus) depends crucially on interactions with the thymic stroma, mainly composed of thymic epithelial cells and thymic interstitial cells. Currently available systems to study thymopoiesis, and thymic function in general, are hampered by a lack of understanding of the origins of thymic epithelial cells. In particular, the population of multipotent stem cells that gives rise to multiple thymic epithelial cell lineages has, so far, not been identified.

For the first time, the present invention describes this novel population of thymic epithelial stem cells: a clonogenic population of self-renewing cells that are able to give rise to multiple different epithelial cell lineages within the thymus, including both cortical and medullary thymic epithelial cell lineages.

The thymic epithelial stem cells identified by the inventors are characterised by their unique protein expression pattern and genetic signature. Of note, the thymic epithelial stem cells may be prospectively isolated based on the expression (or lack thereof) of surface molecules such as BCAM, CD49F, CD90 and CD24. Thymic epithelial stem cells also uniquely demonstrate a “polykeratin” signature, wherein the cells co-express cytokeratin genes that, individually, are typically restricted to specific cell types and are not co-expressed with other cytokeratins.

Methods for isolation of the thymic epithelial stem cell population from thymic tissue are described herein, for the first time. Once isolated from thymus, the described thymic epithelial stem cells are capable of long-term expansion ex vivo. The thymic epithelial stem cells are multipotent and are able to differentiate into multiple specialised thymic cells in vitro.

The described methods for isolating thymic epithelial stem cells, and use of the cells themselves, provides a powerful new research tool for understanding thymus development and for drug screening, for example for agents that can impact cell senescence or proliferation. Isolated thymic epithelial stem cell populations also hold enormous potential as a therapeutic agent, and may find utility in a range of applications, particularly those associated with immune dysfunction, or other disorders associated with impaired or altered thymus activity.

### Definitions

Below are provided certain definitions of terms, technical means, and embodiments used herein.

As used herein, the term “administration” refers to the administration of a composition to a subject. Administration to an animal subject (e.g., to a human) may be by any appropriate route. For example, in some embodiments, administration may be bronchial (including by bronchial instillation), buccal, enteral, intra-arterial, intra-dermal, intra-gastric, intra-medullary, intra-muscular, intra-nasal, intra-peritoneal, intra-thecal, intra-venous, intra-ventricular, within a specific organ or tissue (e.g. intra-hepatic, intra-tumoral, peri-tumoral, etc), mucosal, nasal, oral, rectal, subcutaneous, sublingual, topical, tracheal (including by intra-tracheal instillation), transdermal, vaginal and vitreal. The administration may involve intermittent dosing. Alternatively, administration may involve continuous dosing (e.g., perfusion) for at least a selected period of time.

In some embodiments, the invention provides a cell culture composition. For example, methods of isolating and/or culturing the thymic epithelial stem cells may provide a population of cultured cells. The population of cultured cells may be stored (e.g. frozen) for future use. In some embodiments, the invention provides a method of cell banking. The invention also provides a method of making a cell bank comprising thymic epithelial stem cells. For example, a method of cell banking may include the steps of isolating the thymic epithelial stem cells from thymic tissue, culturing or expanding the cells to obtain a suitable number of cells, and storing (e.g. freezing) the cells for future use. The method may also comprise culturing thymic epithelial stem cells that have been previously isolated (e.g. cells that have been previously isolated and stored in a cell bank). The cell bank comprising thymic epithelial stem cells may provide a source for making a pharmaceutical composition. Thymic epithelial stem cells may be used directly from the bank, or expanded prior to use.

As used herein, the term “cell replacement therapy” refers to . . . e.g. thymic removal early in life associated with negative outcomes, replacement could improve this

As used herein, the term “clonogenic” refers to a cell or cells that is/are capable of single cell cloning, i.e. the ability of a single cell to grow into a colony. The isolated thymic epithelial stem cells of the invention may be clonogenic cells. For example, isolated CD49F^{pos}CD90^{pos}CD24^{neg} thymic epithelial stem cells give rise to expanding colonies in vitro, as described in the Examples.

Clonogenicity may be assessed by any suitable methods as described herein or known in the art, for example by clonogenic assay or colony formation assay.

### Cytokeratins

Cytokeratins or keratins (KRTs) are the intermediate filament proteins that under physiological conditions define the specific lineage differentiation of simple, stratified, or glandular epithelial cell types in different tissues. Cells of the invention surprisingly express multiple cytokeratins, and may be referred to as “polykeratins” or a “polykeratin cell”. As discussed in the Examples, thymic epithelial stem cells were surprisingly found to co-express cytokeratins which would typically only be expressed by one specific cell type. For example, thymic epithelial stem cells were surprisingly found to co-express both KRT8 (typically expressed by simple epithelia) and KRT5 (typically expressed by stratified epithelia).

In some embodiments, the isolated thymic epithelial stem cell expresses at least one, at least two, at least three, at least four, at least five, at least six, at least seven, at least eight, at least nine, or at least ten cytokeratin genes. In some embodiments, the isolated thymic epithelial stem cell expresses at least one cytokeratin gene. In some embodiments, the isolated thymic epithelial stem cell expresses at least two cytokeratin genes. In some embodiments, the isolated thymic epithelial stem cell expresses at least three cytokeratin genes. In some embodiments, the isolated thymic epithelial stem cell expresses at least four cytokeratin genes. In some embodiments, the isolated thymic epithelial stem cell expresses at least five cytokeratin genes. In some embodiments, the isolated thymic epithelial stem cell expresses at least six cytokeratin genes. In some embodiments, the isolated thymic epithelial stem cell expresses at least seven cytokeratin genes. In some embodiments, the isolated thymic epithelial stem cell expresses at least eight cytokeratin genes. In some embodiments, the isolated thymic epithelial stem cell expresses at least nine cytokeratin genes. In some embodiments, the isolated thymic epithelial stem cell expresses at least ten cytokeratin genes.

Cytokeratin or keratin genes expressed by the cells of the invention may include KRT5, KRT8, KRT13, KRT14, KRT15, KRT17, KRT18, and KRT19, or combinations thereof.

In some embodiments, the isolated epithelial stem cell expresses KRT5. In some embodiments, the isolated epithelial stem cell expresses KRT8. In some embodiments, the isolated epithelial stem cell expresses KRT13. In some embodiments, the isolated epithelial stem cell expresses KRT14. In some embodiments, the isolated epithelial stem cell expresses KRT15. In some embodiments, the isolated epithelial stem cell expresses KRT17. In some embodiments, the isolated epithelial stem cell expresses KRT18. In some embodiments, the isolated epithelial stem cell expresses KRT19.

In some embodiments, the isolated epithelial stem cell expresses KRT13, KRT18 and KRT15. In some embodiments, the isolated epithelial stem cell expresses KRT13, KRT18 and KRT17. In some embodiments, the isolated epithelial stem cell expresses KRT5, KRT14, KRT18 and KRT13. In some embodiments, the isolated epithelial stem cell expresses KRT5, KRT18 and KRT13. In some embodiments, the isolated epithelial stem cell expresses, KRT14, KRT18 and KRT13. In some embodiments, the isolated epithelial stem cell expresses KRT14, KRT17 and KRT13. In some embodiments, the isolated epithelial stem cell expresses KRT14, KRT17 and KRT18. In some embodiments, the isolated epithelial stem cell expresses KRT15, KRT17 and KRT13. In some embodiments, the isolated epithelial stem cell expresses KRT15, KRT17 and KRT18.

In some embodiments, the isolated epithelial stem cell does not express KRT7, KRT1, KRT10, KRT4, KRT16, KRT23, KRT6A, or combinations thereof. In some embodiments, the isolated epithelial stem cell does not express KRT7. In some embodiments, the isolated epithelial stem cell does not express KRT1. In some embodiments, the isolated epithelial stem cell does not express KRT10. In some embodiments, the isolated epithelial stem cell does not express KRT4. In some embodiments, the isolated epithelial stem cell does not express KRT16. In some embodiments, the isolated epithelial stem cell does not express KRT23. In some embodiments, the isolated epithelial stem cell does not express KRT6A.

Determining the expression of cytokeratin genes may be performed by any suitable method known in the art and as discussed herein.

As used herein, the term “differentiation” refers to the ability of a dividing cell (for example, an isolated thymic epithelial stem cell) to change its functional or phenotypical type, to give rise to a different cell type.

The isolated thymic epithelial stem cells have been surprisingly shown to be capable of differentiation into both cortical and medullary thymic epithelial cells, irrespective of whether the initial cell was a cortical thymic epithelial stem cell or a medullary thymic epithelial stem cell.

In some embodiments, the isolated thymic epithelial stem cell is capable of differentiating into a medullary thymic epithelial cell. Exemplary medullary thymic epithelial cells (mTEC) include mTEC progenitors, neuroendocrine cells, myoid cells, ionocytes and Hassall body region cells. In some embodiments, mTECs express CLDN3, CLDN4 and/or ASCL1, or combinations thereof.

In some embodiments, the isolated thymic epithelial stem cell is capable of differentiating into a cortical thymic epithelial cell. Cortical thymic epithelial cells include cells that express CD205, CTSV, FOXN1, KCNIP13, CD274 (PDL-1) or combinations thereof.

The invention further provides methods of differentiating thymic epithelial stem cells. In some embodiments, the methods are in vitro methods. In some embodiments, the method of differentiating an isolated thymic epithelial stem cell comprises:
(a) providing at least one isolated thymic epithelial stem cell,
(b) contacting the at least one isolated thymic epithelial stem cell to a membrane, wherein the at least one isolated thymic epithelial stem cell is in contact with an upper surface of the membrane,
(c) providing a cell culture medium, wherein the cell culture medium is positioned below a lower surface of the membrane.

The cell culture medium is any cell culture medium suitable for supporting growth and maintenance of cell cultures in vitro. Suitable cell culture media are known in the art. In some embodiments, the cell culture medium is Pneumacult Maintenance Medium™. In some embodiments, the cell culture medium is Neurocult™ Base, for example a cell culture medium comprising Neurobasal™ Medium Gibco B-27™ Plus Supplement (50×) supplemented with B-27™ Plus Supplement (Gibco, 50×); N-2 Supplement (Gibco, 100×); DAPT 10 uM. In another example, a suitable cell culture medium comprises DMEM-F12 (3:1); Penicillin/streptomycin (P/S), 10% Serum; Insulin and Triiodothyronine. In some embodiments, the cell culture medium is any basal culture medium (for example DMEM:F12) which can be complemented with serum (2-10%) and other supplements favouring cell survival and proliferation (for example growth factors and/or hormones). Alternatively basal medium, can be combined with supplements that provide also basic nutrients (for example, serum-free media).

In some embodiments, the at least one isolated thymic epithelial stem cell is expanded or cultured as described herein prior to differentiating.

In one aspect, the present invention provides a cell culture comprising at least one isolated thymic epithelial stem cell of the invention.

In some embodiments, the invention provides a method of differentiating an isolated thymic epithelial stem cell, comprising:
(a) providing at least one isolated thymic epithelial stem cell,
(b) contacting the at least one isolated thymic epithelial stem cell to a membrane, wherein the at least one isolated thymic epithelial stem cell is in contact with an upper surface of the membrane,
(c) contacting the at least one isolated thymic epithelial stem cell and the membrane with a first cell culture medium,
(d) maintaining the at least one isolated thymic epithelial stem cell under conditions suitable for expansion of the isolated thymic epithelial stem cell to provide an expanded population of thymic epithelial stem cells,
(d) removing the first cell culture medium,
(e) providing a second cell culture medium, wherein the second cell culture medium is positioned below a lower surface of the membrane, and
(f) maintaining the expanded population of thymic epithelial stem cells under conditions suitable for differentiation of the expanded population of thymic epithelial stem cells.

In some embodiments, the first cell culture medium is any medium suitable to support the growth of epithelial cells in culture. In some embodiments, the first cell culture medium is serum-free medium. In some embodiments, the first cell culture medium is cFAD medium. In some embodiments the second cell culture medium is Pneumacult Maintenance Medium™. In some embodiments, the second cell culture medium is Neurocult™ Base, as described above.

As used herein, the term “self-renewal” or “ex vivo self-renewal” refers to the capability of a cell, for example a thymic epithelial stem cell, to give rise to more cells of the same type. Generally, self-renewal indicates that a cell is able to self-renew by dividing. Ex vivo self-renewal describes a cell that is capable of self-renewal once isolated or otherwise removed from the in vivo environment, for example once placed in culture.

### Gene Expression

The expression of a gene or gene set may be used to detect, identify or distinguish cell types from one another. The inventors have surprisingly discovered a novel gene expression profile that can distinguish thymic epithelial stem cells from other cell types. This gene expression profile can be used to identify thymic epithelial stem cells as well as in methods of isolating and culturing thymic epithelial stem cells.

In some embodiments, isolated thymic epithelial stem cells express at least one gene selected from the group consisting of EPCAM, CD49F, FN1, TIMP1, IFITM3, VCAM1, BCAM, LIFR, CEPBD, CLU, CCL19, CH25H, COL7A1, CTGF, APOE, FGFR2, BOC, ITGA5, SOX17, YAP1, PTGDS, CD34, VWF, SPARC, CAV-1, EPAS-1, TIMP3, COL4A2, COL5A1, COL6A3, TP63 (for example ΔNTP63α) and cMYC and combinations thereof. In some embodiments, isolated thymic epithelial stem cells express EPCAM. In some embodiments, isolated thymic epithelial stem cells express CD49F. In some embodiments, isolated thymic epithelial stem cells express FN1. In some embodiments, isolated thymic epithelial stem cells express IFITM3. In some embodiments, isolated thymic epithelial stem cells express VCAM1. In some embodiments, isolated thymic epithelial stem cells express BCAM. In some embodiments, isolated thymic epithelial stem cells express CEPBD. In some embodiments, isolated thymic epithelial stem cells express CLU. In some embodiments, isolated thymic epithelial stem cells express CCL19. In some embodiments, isolated thymic epithelial stem cells express CH25H. In some embodiments, isolated thymic epithelial stem cells express COL7A1. In some embodiments, isolated thymic epithelial stem cells express CTGF. In some embodiments, isolated thymic epithelial stem cells express APOE. In some embodiments, isolated thymic epithelial stem cells express PTGDS. In some embodiments, isolated thymic epithelial stem cells express FGFR2. In some embodiments, isolated thymic epithelial stem cells express BOC. In some embodiments, isolated thymic epithelial stem cells express ITGA5. In some embodiments, isolated thymic epithelial stem cells express SOX17. In some embodiments, isolated thymic epithelial stem cells express LIFR. In some embodiments, isolated thymic epithelial stem cells express YAP1. In some embodiments, isolated thymic epithelial stem cells express PTGDS. In some embodiments, isolated thymic epithelial stem cells express CD34. In some embodiments, isolated thymic epithelial stem cells express VWF. In some embodiments, isolated thymic epithelial stem cells express SPARC. In some embodiments, isolated thymic epithelial stem cells express CAV-1. In some embodiments, isolated thymic epithelial stem cells express EPAS-1. In some embodiments, isolated thymic epithelial stem cells express TIMP3. In some embodiments, isolated thymic epithelial stem cells express COL4A2. In some embodiments, isolated thymic epithelial stem cells express COL5A1. In some embodiments, isolated thymic epithelial stem cells express COL6A3. In some embodiments, isolated thymic epithelial stem cells express TP63. In some embodiments, isolated thymic epithelial stem cells express ΔNTP63α.

In some embodiments, isolated thymic epithelial stem cells express EPCAM, CD49F and FN1. In some embodiments, isolated thymic epithelial stem cells express EPCAM, CD49F and IFITM3. In some embodiments, isolated thymic epithelial stem cells express CD49F, FN1 and TIMP1. In some embodiments, isolated thymic epithelial stem cells express COL7A1 and CTGF. In some embodiments, isolated thymic epithelial stem cells express FN1, IFITM3 and TIMP1. In some embodiments, isolated thymic epithelial stem cells express EPCAM and CD49F. In some embodiments, isolated thymic epithelial stem cells express EPCAM and BCAM. In some embodiments, isolated thymic epithelial stem cells express a cytokeratin (KRT) and CD49F. In some embodiments, isolated thymic epithelial stem cells express a cytokeratin (KRT) and BCAM. In some embodiments, isolated thymic epithelial stem cells express at least one gene selected from the group consisting of TP63 (for example ΔNTP63α) and KRT15, or combinations thereof. In some embodiments, isolated thymic epithelial stem cells express a cytokeratin (KRT) and TP63. In some embodiments, isolated thymic epithelial stem cells express a cytokeratin (KRT) and ΔNTP63α. In some embodiments, isolated thymic epithelial stem cells express a KRT15 and TP63. In some embodiments, isolated thymic epithelial stem cells express KRT15 and ΔNTP63α. In some embodiments, isolated thymic epithelial stem cells express a KRT15 and BCAM. In some embodiments, isolated thymic epithelial stem cells express a KRT15, BCAM and TP63. In some embodiments, isolated thymic epithelial stem cells express KRT15, BCAM and ΔNTP63α. In some embodiments, isolated thymic epithelial stem cells express a KRT8 and TP63. In some embodiments, isolated thymic epithelial stem cells express KRT8 and ΔNTP63α. In some embodiments, isolated thymic epithelial stem cells express a KRT8 and BCAM. In some embodiments, isolated thymic epithelial stem cells express a KRT8, BCAM and TP63. In some embodiments, isolated thymic epithelial stem cells express KRT8, BCAM and ΔNTP63α. In some embodiments, isolated thymic epithelial stem cells express a KRT5 and TP63. In some embodiments, isolated thymic epithelial stem cells express KRT5 and ΔNTP63α. In some embodiments, isolated thymic epithelial stem cells express a KRT5 and BCAM. In some embodiments, isolated thymic epithelial stem cells express a KRT5, BCAM and TP63. In some embodiments, isolated thymic epithelial stem cells express KRT5, BCAM and ΔNTP63α.

In some embodiments, isolated thymic epithelial stem cells express at least one gene selected from the group consisting of FN1, TIMP1, IFITM3, VCAM1, BCAM, LIFR, CEPBD, CLU, or combinations thereof. In some embodiments, isolated thymic epithelial stem cells express at least one gene selected from the group consisting of FN1 and TIMP1 or combinations thereof. In some embodiments, isolated thymic epithelial stem cells express at least one gene selected from the group consisting of FN1 and IFITM3 or combinations thereof. In some embodiments, isolated thymic epithelial stem cells express at least one gene selected from the group consisting of FN1 and VCAM1 or combinations thereof. In some embodiments, isolated thymic epithelial stem cells express at least one gene selected from the group consisting of FN1 and BCAM or combinations thereof. In some embodiments, isolated thymic epithelial stem cells express at least one gene selected from the group consisting of FN1 and LIFR or combinations thereof. In some embodiments, isolated thymic epithelial stem cells express at least one gene selected from the group consisting of FN1 and CEPBD or combinations thereof. In some embodiments, isolated thymic epithelial stem cells express at least one gene selected from the group consisting of FN1 and CLU or combinations thereof. In some embodiments, isolated thymic epithelial stem cells express at least one gene selected from the group consisting of TIMP1 and IFITM3 or combinations thereof. In some embodiments, isolated thymic epithelial stem cells express at least one gene selected from the group consisting of TIMP1 and VCAM1 or combinations thereof. In some embodiments, isolated thymic epithelial stem cells express at least one gene selected from the group consisting of TIMP1 and BCAM or combinations thereof. In some embodiments, isolated thymic epithelial stem cells express at least one gene selected from the group consisting of TIMP1 and LIFR or combinations thereof. In some embodiments, isolated thymic epithelial stem cells express at least one gene selected from the group consisting of TIMP1 and CEPBD or combinations thereof. In some embodiments, isolated thymic epithelial stem cells express at least one gene selected from the group consisting of TIMP1 and CLU or combinations thereof. In some embodiments, isolated thymic epithelial stem cells express at least one gene selected from the group consisting of IFITM3 and VCAM1 or combinations thereof. In some embodiments, isolated thymic epithelial stem cells express at least one gene selected from the group consisting of IFITM3 and BCAM or combinations thereof. In some embodiments, isolated thymic epithelial stem cells express at least one gene selected from the group consisting of IFITM3 and LIFR or combinations thereof. In some embodiments, isolated thymic epithelial stem cells express at least one gene selected from the group consisting of IFITM3 and CEPBD or combinations thereof. In some embodiments, isolated thymic epithelial stem cells express at least one gene selected from the group consisting of IFITM3 and CLU or combinations thereof. In some embodiments, isolated thymic epithelial stem cells express at least one gene selected from the group consisting of VCAM1 and BCAM or combinations thereof. In some embodiments, isolated thymic epithelial stem cells express at least one gene selected from the group consisting of VCAM1 and LIFR or combinations thereof. In some embodiments, isolated thymic epithelial stem cells express at least one gene selected from the group consisting of VCAM1 and CEPBD or combinations thereof. In some embodiments, isolated thymic epithelial stem cells express at least one gene selected from the group consisting of VCAM1 and CLU or combinations thereof. In some embodiments, isolated thymic epithelial stem cells express at least one gene selected from the group consisting of BCAM and LIFR or combinations thereof. In some embodiments, isolated thymic epithelial stem cells express at least one gene selected from the group consisting of BCAM and CEPBD or combinations thereof. In some embodiments, isolated thymic epithelial stem cells express at least one gene selected from the group consisting of BCAM and CLU or combinations thereof. In some embodiments, isolated thymic epithelial stem cells express at least one gene selected from the group consisting of LIFR and CEPBD or combinations thereof. In some embodiments, isolated thymic epithelial stem cells express at least one gene selected from the group consisting of LIFR and CLU or combinations thereof. In some embodiments, isolated thymic epithelial stem cells express at least one gene selected from the group consisting of CEPBD and CLU or combinations thereof.

In some embodiments, thymic epithelial stem cells express one or more cytokeratin genes, as discussed above.

In some embodiments, isolated thymic epithelial stem cells express at least one gene selected from the group consisting of KRT15, FN1 and TIMP1 or combinations thereof. In some embodiments, isolated thymic epithelial stem cells express at least one gene selected from the group consisting of KRT15, FN1 and IFITM3 or combinations thereof. In some embodiments, isolated thymic epithelial stem cells express at least one gene selected from the group consisting of KRT15, FN1 and VCAM1 or combinations thereof. In some embodiments, isolated thymic epithelial stem cells express at least one gene selected from the group consisting of KRT15, FN1 and BCAM or combinations thereof. In some embodiments, isolated thymic epithelial stem cells express at least one gene selected from the group consisting of KRT15, FN1 and LIFR or combinations thereof. In some embodiments, isolated thymic epithelial stem cells express at least one gene selected from the group consisting of KRT15, FN1 and CEPBD or combinations thereof. In some embodiments, isolated thymic epithelial stem cells express at least one gene selected from the group consisting of KRT15, FN1 and CLU or combinations thereof. In some embodiments, isolated thymic epithelial stem cells express at least one gene selected from the group consisting of KRT15, TIMP1 and IFITM3 or combinations thereof. In some embodiments, isolated thymic epithelial stem cells express at least one gene selected from the group consisting of KRT15, TIMP1 and VCAM1 or combinations thereof. In some embodiments, isolated thymic epithelial stem cells express at least one gene selected from the group consisting of KRT15, TIMP1 and BCAM or combinations thereof. In some embodiments, isolated thymic epithelial stem cells express at least one gene selected from the group consisting of KRT15, TIMP1 and LIFR or combinations thereof. In some embodiments, isolated thymic epithelial stem cells express at least one gene selected from the group consisting of KRT15, TIMP1 and CEPBD or combinations thereof. In some embodiments, isolated thymic epithelial stem cells express at least one gene selected from the group consisting of KRT15, TIMP1 and CLU or combinations thereof. In some embodiments, isolated thymic epithelial stem cells express at least one gene selected from the group consisting of KRT15, IFITM3 and VCAM1 or combinations thereof. In some embodiments, isolated thymic epithelial stem cells express at least one gene selected from the group consisting of KRT15, IFITM3 and BCAM or combinations thereof. In some embodiments, isolated thymic epithelial stem cells express at least one gene selected from the group consisting of KRT15, IFITM3 and LIFR or combinations thereof. In some embodiments, isolated thymic epithelial stem cells express at least one gene selected from the group consisting of KRT15, IFITM3 and CEPBD or combinations thereof. In some embodiments, isolated thymic epithelial stem cells express at least one gene selected from the group consisting of KRT15, IFITM3 and CLU or combinations thereof. In some embodiments, isolated thymic epithelial stem cells express at least one gene selected from the group consisting of KRT15, VCAM1 and BCAM or combinations thereof. In some embodiments, isolated thymic epithelial stem cells express at least one gene selected from the group consisting of KRT15, VCAM1 and LIFR or combinations thereof. In some embodiments, isolated thymic epithelial stem cells express at least one gene selected from the group consisting of KRT15, VCAM1 and CEPBD or combinations thereof. In some embodiments, isolated thymic epithelial stem cells express at least one gene selected from the group consisting of KRT15, VCAM1 and CLU or combinations thereof. In some embodiments, isolated thymic epithelial stem cells express at least one gene selected from the group consisting of KRT15, BCAM and LIFR or combinations thereof. In some embodiments, isolated thymic epithelial stem cells express at least one gene selected from the group consisting of KRT15, BCAM and CEPBD or combinations thereof. In some embodiments, isolated thymic epithelial stem cells express at least one gene selected from the group consisting of KRT15, BCAM and CLU or combinations thereof. In some embodiments, isolated thymic epithelial stem cells express at least one gene selected from the group consisting of KRT15, LIFR and CEPBD or combinations thereof. In some embodiments, isolated thymic epithelial stem cells express at least one gene selected from the group consisting of KRT15, LIFR and CLU or combinations thereof. In some embodiments, isolated thymic epithelial stem cells express at least one gene selected from the group consisting of KRT15, CEPBD and CLU or combinations thereof.

In some embodiments, isolated thymic epithelial stem cells as described herein express genes associated with a “holoclone” signature. A “holoclone” signature represents an activated/proliferating, long-term expanding stem cell, as defined in the art [6]. In some embodiments, isolated thymic epithelial stem cells as described herein express at least one gene selected from the group consisting of CCNA2, AURKB, FOXM1, ANLN, LMNB1, HMGB2, or combinations thereof. In some embodiments, isolated thymic epithelial stem cells as described herein may be activated. In some embodiments, activated isolated thymic epithelial stem cells as described herein express at least one selected from the group consisting of CCNA2, AURKB, FOXM1, ANLN, LMNB1, HMGB2, or combinations thereof.

Gene expression may be detected or measured by any suitable method known in the art. For example, gene expression may be measured by polymerase chain reaction (PCR), fluorescent in situ hybridisation (FISH), single cell RNA sequencing (scRNA-seq), spatial transcriptomics, RNA-scope and HiPLEX.

### Kits

The invention also provides a kit for identifying a thymic epithelial stem cell as described herein. In some embodiments, the kit comprises at least one binding molecule that binds to BCAM, CD49F, CD90 or CD24.

In some embodiments, the kit comprises a binding molecule that binds to BCAM. In some embodiments, the kit comprises a binding molecule that binds to CD49F. In some embodiments, the kit comprises a binding molecule that binds to CD90. In some embodiments, the kit comprises a binding molecule that binds to CD24. In some embodiments, the kit comprises a first binding molecule that binds to BCAM and a second binding molecule that binds to CD49F. In some embodiments, the kit comprises a first binding molecule that binds to BCAM and a second binding molecule that binds to CD90. In some embodiments, the kit comprises a first binding molecule that binds to BCAM and a second binding molecule that binds to CD24. In some embodiments, the kit comprises a first binding molecule that binds to CD49F and a second binding molecule that binds to CD90. In some embodiments, the kit comprises a first binding molecule that binds to CD49F and a second binding molecule that binds to CD24. In some embodiments, the kit comprises a first binding molecule that binds to CD90 and a second binding molecule that binds to CD24. In some embodiments, the kit comprises a first binding molecule that binds to BCAM, a second binding molecule that binds to CD49F, and a third binding molecule that binds to CD90. In some embodiments, the kit comprises a first binding molecule that binds to BCAM, a second binding molecule that binds to CD49F, and a third binding molecule that binds to CD24. In some embodiments, the kit comprises a first binding molecule that binds to CD49F, a second binding molecule that binds to CD90, and a third binding molecule that binds to CD24. In some embodiments, the kit comprises a first binding molecule that binds to BCAM, a second binding molecule that binds to CD49F, a third binding molecule that binds to CD90, and a fourth binding molecule that binds to CD24.

In some embodiments, the binding molecule is an antibody or fragment thereof. In some embodiments, the binding molecule is conjugated with or labelled with a detectable agent. In some embodiments, the detectable agent is a fluorophore.

The kit may be a kit for identifying a thymic epithelial stem cell as described herein using fluorescence activated cell sorting (FACS). Exemplary binding molecules and fluorophores are provided in Table 1 below.

TABLE 1
Antigens and fluorochromes

| Antigen | Fluorochrome | Dilution | Clone |
|---|---|---|---|
| BCAM | BV421 | 1:100 | MSK19 |
| CD24 | APC | 1:100 | ML5 |
| CD24 | BV605 | 1:100 | ML5 |
| CD205 | PE | 1:400 | HD30 |
| CD235ab (glycophorin | Biotin | 1:100 | HIR2 |
| A and B) | | | |
| CD45 | Biotin | 1:100 | a30-F11 |
| CD45 | APC | 1:200 | HI30 |
| CD49F | AF488 | 1:200 | GoH3 |
| CD49F | BV421 | 1:200 | GoH3 |
| CD90 | AF700 | 1:200 | 5E10 |
| CD90 | FITC | 1:200 | 5E10 |
| EpCAM (CD326) | PE-Cy7 | 1:100 | 9C4 |
| VCAM1 (CD106) | BV421 | 1:100 | STA |
| Feeder | PE | 1:100 | RMV-7 |
| Zombie Aqua ™ | | 1:200 | |
| Fixable Viability Kit | | | |

In some embodiments, the kit comprises instructions for identifying a thymic epithelial stem cell as described herein.

### Long-Term Expansion Capacity

Cell culture generally involves serial passaging of cells in order to grow a cell line or cell population to a particular number of cells. Expansion (i.e. the increase in cell number) generally occurs through division of the cells in culture. Long-term expansion capacity, as used herein, describes the ability of thymic epithelial stem cells to expand in vitro for a number of passages or population doublings, before reaching senescence, for example, without losing their stem cell phenotype or ability to divide.

In some embodiments, the isolated thymic epithelial stem cell is capable of at least 5 population doublings in vitro. In some embodiments, the isolated thymic epithelial stem cell is capable of at least population doublings in vitro. In some embodiments, the isolated thymic epithelial stem cell is capable of at least 15 population doublings in vitro. In some embodiments, the isolated thymic epithelial stem cell is capable of at least 20 population doublings in vitro. In some embodiments, the isolated thymic epithelial stem cell is capable of at least 25 population doublings in vitro. In some embodiments, the isolated thymic epithelial stem cell is capable of at least 30 population doublings in vitro.

### Methods of Treatment

The isolated thymic epithelial stem cells as described herein, as well as related cell culture compositions, pharmaceutical compositions and thymic constructs, may be useful in methods of treating a disease or disorder in a subject.

In some embodiments, the invention provides a method of treating a disease or disorder in a subject, comprising administering to the subject an isolated thymic epithelial stem cell, a cell culture composition, a pharmaceutical composition or a thymic construct, as described herein.

### Surface Markers

In some embodiments, the cell surface markers or genes discussed herein comprise or consist of sequences as defined in the art, for example in sequence accession databases. In some embodiments, the cell surface markers or genes discussed herein comprise or consist of one or more amino acid sequences as set out in Table 2 below. Further details of exemplary identities and information available in the art for the surface markers is set out in Table 2.

TABLE 2
Cell surface markers or genes, descriptions and accession numbers

| | | Human protein | Mouse protein |
|---|---|---|---|
| Cell surface | | Refseq | Refseq |
| marker | | accession | accession |
| or gene | Description | number(s) | number(s) |
| BCAM | Also known as | NP_001013275 | NP_065232 |
| | CD239 | NP_005572 | |
| CD24 | Also known as | NP_001278666 | N/A |
| | heat stable | NP_001278667 | |
| | antigen (HSA) | NP_001278668 | |
| | CD24 | NP_037362 | |
| | | NP_001346013 | |
| CD205 | Also known as | NP_001185688 | NP_038853 |
| | LY75 | NP_001185689 | |
| CD235ab | Also known as | NP_001295116 | |
| | glycophorin A | NP_001295119 | |
| | and B | NP_002090 | |
| CD45 | Also known as | NP_001254727 | NP_001104786 |
| | PTPRC or LCA | NP_002829 | NP_001255215 |
| | | NP_563578 | NP_035340 |
| | | NP_563578.2 | |
| | | NP_002829.3 | |
| CD49F | Also known as | NP_000201 | NP_001264899 |
| | ITGA6, VLA-6, | NP_001073286 | NP_032423 |
| | integrin subunit | NP_001303235 | |
| | alpha 6 | NP_001352458 | |
| | | NP_001352459 | |
| CD90 | Also known as | NP_001298089 | NP_033408 |
| | THY1, CDw90, | NP_001298091 | |
| | Thy-1 cell surface | NP_006279 | |
| | antigen | NP_001358979 | |
| EpCAM | Also known as | NP_002345 | NP_032558 |
| (CD326) | DIAR5, EGP-2, | | |
| | EGP314, EGP40, | | |
| | ESA, HNPCC8, | | |
| | KS1/4, KSA, | | |
| | M4S1, MIC18, | | |
| | MK-1, TACSTD1, | | |
| | TROP1, epithelial | | |
| | cell adhesion | | |
| | molecule, | | |
| | BerEp4, MOC-31, | | |
| | Ber-Ep4 | | |
| VCAM1 | Also known as | NP_001069 | NP_035823 |
| (CD106) | INCAM-100, | NP_001186763 | |
| | vascular cell | NP_542413 | |
| | adhesion | | |
| | molecule 1 | | |
| FN1 | Also known as | NP_001293058 | NP_001263337 |
| | CIG, ED-B, FINC, | NP_001293059 | NP_001263338 |
| | FN, FNZ, GFND, | NP_001293060 | NP_001263339 |
| | GFND2, LETS, | NP_001293061 | NP_001263340 |
| | MSF, fibronectin | NP_002017 | NP_001263341 |
| | 1, SMDCF | | |
| TIMP1 | Also known as | NP_003245 | NP_001037849 |
| | CLGI, EPA, EPO, | NP_003245.1 | NP_001281209 |
| | HCI, TIMP, TIMP | | NP_035723 |
| | metallopeptidase | | |
| | inhibitor 1 | | |
| IFITM3 | Also known as | NP_066362 | NP_079654 |
| | IFITM3, 1-8U, | | |
| | DSPA2b, IP15, | | |
| | interferon induced | | |
| | transmembrane | | |
| | protein 3 | | |
| CEPBD | CCAAT enhancer | NP_005186.2 | |
| | binding protein | | |
| | delta | | |
| CLU | Also known as | NP_001822 | NP_038520 |
| | AAG4, APO-J, | | |
| | APOJ, CLI, | | |
| | CLU1, CLU2, | | |
| | KUB1, NA1/NA2, | | |
| | SGP-2, SGP2, | | |
| | SP-40, TRPM-2, | | |
| | TRPM2, clusterin | | |
| CCL19 | Also known as | NP_006265 | |
| | CKb11, ELC, | | |
| | MIP-3b, MIP3B, | | |
| | SCYA19, C-C | | |
| | motif chemokine | | |
| | ligand 19 | | |
| CH25H | Cholesterol 25- | NP_003947.1 | |
| | hydroxylase | | |
| COL7A1 | Also known as | NP_000085 | NP_031764 |
| | EBD1, EBDCT, | | |
| | EBR1, NDNC8, | | |
| | collagen type VII | | |
| | alpha 1, collagen | | |
| | type VII alpha 1 | | |
| | chain | | |
| CTGF | Also known as | NP_001892 | NP_034347 |
| | CCN2, HCS24, | | |
| | IGFBP8, NOV2, | | |
| | connective tissue | | |
| | growth factor, | | |
| | cellular | | |
| | communication | | |
| | network factor 2, | | |
| | CTGF | | |
| APOE | Also known as | NP_000032 | NP_001292748 |
| | AD2, APO-E, | NP_001289617 | NP_001292772 |
| | LDLCQ5, LPG, | NP_001289618 | NP_001292773 |
| | apolipoprotein E, | NP_001289619 | NP_033826 |
| | ApoE4 | NP_001289620 | |
| FGFR2 | Also known as | NP_000132 | NP_001334567 |
| | BBDS, BEK, | NP_001138385 | NP_034337 |
| | BFR-1, CD332, | NP_001138386 | NP_963895 |
| | CEK3, CFD1, | NP_001138387 | |
| | ECT1, JWS, K- | NP_001138388 | |
| | SAM, KGFR, | | |
| | TK14, TK25, | | |
| | fibroblast growth | | |
| | factor receptor 2 | | |
| BOC | Also known as | NP_001288790 | NP_766094 |
| | CDON2, BOC cell | NP_001288796 | |
| | adhesion | NP_150279 | |
| | associated, | NP_001365002 | |
| | oncogene | NP_001365003 | |
| | regulated, Boi | | |
| ITGA5 | Also known as | NP_002196 | NP_001300970 |
| | CD49e, FNRA, | | NP_034707 |
| | VLA5A, VLA-5, | | |
| | integrin subunit | | |
| | alpha 5 | | |
| SOX17 | Also known as | NP_071899 | NP_001276393 |
| | VUR3, SRY-box | | NP_001276394 |
| | 17, SRY-box | | NP_001276395 |
| | transcription | | NP_001276396 |
| | factor 17 | | NP_035571 |
| LIFR | Also known as | NP_001121143 | NP_001106857 |
| | CD118, LIF-R, | NP_002301 | NP_038612 |
| | SJS2, STWS, | NP_001351226 | NP_001345522 |
| | SWS, leukemia | NP_001351227 | |
| | inhibitory factor | | |
| | receptor alpha, | | |
| | LIF receptor | | |
| | alpha, LIF | | |
| | receptor subunit | | |
| | alpha | | |
| PTGDS | Also known as | NP_000945 | NP_032989 |
| | L-PGDS, LPGDS, | | |
| | PDS, PGD2, | | |
| | PGDS, PGDS2, | | |
| | prostaglandin D2 | | |
| | synthase | | |
| CCNA2 | Also known as | NP_001228 | NP_033958 |
| | CCN1, CCNA, | | |
| | cyclin A2 | | |
| YAP1 | Also known as | NP_001123617 | NP_001164618 |
| | COB1, YAP, | NP_001181973 | NP_033560 |
| | YAP2, YAP65, | NP_001181974 | |
| | YKI, Yes | NP_001269026 | |
| | associated | NP_001269027 | |
| | protein 1, Yap, | | |
| | Yes1 associated | | |
| | transcriptional | | |
| | regulator | | |
| AURKB | Also known as | NP_001243763 | NP_035626 |
| | AIK2, AIM-1, | NP_001271455 | |
| | AIM1, ARK2, | NP_001300879 | |
| | AurB, IPL1, | NP_001300880 | |
| | PPP1R48, | NP_001300881 | |
| | STK12, STK5, | | |
| | aurkb-sv1, aurkb- | | |
| | sv2, aurora | | |
| | kinase B, STK-1, | | |
| | ARK-2 | | |
| FOXM1 | Also known as | NP_001230017 | |
| | FKHL16, | NP_001230018 | |
| | FOXM1B, HFH-11, | NP_068772 | |
| | HFH11, HNF-3, | NP_973731 | |
| | INS-1, | NP_973732 | |
| | MPHOSPH2, | | |
| | MPP-2, MPP2, | | |
| | PIG29, TGT3, | | |
| | TRIDENT, | | |
| | forkhead box M1, | | |
| | FOXM1A, | | |
| | FOXM1C | | |
| ANLN | Also known as | NP_001271230 | NP_082666 |
| | Scraps, scra, | NP_001271231 | |
| | FSGS8, anillin | NP_061155 | |
| | actin binding | | |
| | protein | | |
| LMNB1 | Also known as | NP_001185486 | NP_034851 |
| | ADLD, LMN, | NP_005564 | |
| | LMN2, LMNB, | | |
| | lamin B1, | | |
| | MCPH26 | | |
| HMGB2 | Also known as | NP_001124160 | NP_032278 |
| | HMG2, high | NP_001124161 | NP_001350372 |
| | mobility group | NP_002120 | NP_001350373 |
| | box 2 | | NP_001350374 |
| KRT5 | Also known as | NP_000415 | NP_081287 |
| | CK5, DDD, | | |
| | DDD1, EBS2, K5, | | |
| | KRT5A, keratin 5, | | |
| | EBS2E, EBS2B, | | |
| | EBS2C, EBS1, | | |
| | EBS2A, EBS2F, | | |
| | EBS2D | | |
| KRT14 | Also known as | NP_000517 | NP_001300885 |
| | CK14, EBS3, | | NP_001300886 |
| | EBS4, K14, NFJ, | | NP_058654 |
| | keratin 14, | | |
| | EBS1D, EBS1, | | |
| | EBS1B, EBS1A, | | |
| | EBS1C | | |
| CD34 | Also known as | NP_001020280 | NP_001104529 |
| | Hematopoietic | NP_001764 | NP_598415 |
| | progenitor cell | | |
| | antigen CD34, | | |
| | CD34 sialomucin | | |
| VWF | Also known as | NP_000543 | NP_035838 |
| | F8VWD, von | | |
| | Willebrand factor | | |
| SPARC | Also known as | NP_001296372 | NP_001277746 |
| | Osteonectin (ON), | NP_001296373 | NP_033268 |
| | secreted protein | NP_003109 | |
| | acidic and rich in | | |
| | cysteine (SPARC), | | |
| | basement-membrane | | |
| | protein 40 (BM- | | |
| | 40), O117, | | |
| | secreted protein | | |
| | acidic and | | |
| | cysteine rich, | | |
| | ONT | | |
| CAV-1 | Also known as | NP_001166366 | NP_001229993 |
| | Caveolin-1, | NP_001166367 | NP_031642 |
| | BSCL3, CGL3, | NP_001166368 | |
| | LCCNS, | NP_001744 | |
| | MSTP085, PPH3, | | |
| | VIP21, Caveolin 1 | | |
| EPAS-1 | Also known as | NP_001421 | NP_034267 |
| | Endothelial PAS | | |
| | domain- | | |
| | containing protein | | |
| | 1, ECYT4, HIF2A, | | |
| | HLF, MOP2, | | |
| | PASD2, | | |
| | bHLHe73, | | |
| | endothelial PAS | | |
| | domain protein 1, | | |
| | Hypoxia-inducible | | |
| | factor-2alpha | | |
| TIMP3 | Also known as | NP_000353 | NP_000353 |
| | Metalloproteinase | | |
| | inhibitor 3, | | |
| | HSMRK222, | | |
| | K222, K222TA2, | | |
| | SFD, TIMP | | |
| | metallopeptidase | | |
| | inhibitor 3 | | |
| COL4A2 | Also known as | NP_001837 | NP_034062 |
| | Collagen alpha- | | |
| | 2(IV) chain, ICH, | | |
| | POREN2, | | |
| | collagen type IV | | |
| | alpha 2, collagen | | |
| | type IV alpha 2 | | |
| | chain, BSVD2 | | |
| COL5A1 | Also known as | NP_000084 | NP_056549 |
| | Collagen alpha- | NP_001265003 | |
| | 1(V) chain, | | |
| | EDSC, collagen | | |
| | type V alpha 1, | | |
| | collagen type V | | |
| | alpha 1 chain, | | |
| | EDSCL1, FMDMF | | |
| COL6A3 | Also known as | NP_004360 | N/A |
| | Collagen alpha- | NP_476505 | |
| | 3(VI) chain, | NP_476506 | |
| | DYT27, BTHLM1, | NP_476507 | |
| | UCMD1, collagen | NP_476508 | |
| | type VI alpha 3, | | |
| | collagen type VI | | |
| | alpha 3 chain | | |
| cMYC | Also known as | NP_002458 | NP_001170823 |
| | MYC, MRTL, | NP_001341799 | NP_001170824 |
| | MYCC, bHLHe39, | | NP_001170825 |
| | c-Myc, v-myc | | NP_034979 |
| | avian | | |
| | myelocytomatosis | | |
| | viral oncogene | | |
| | homolog, MYC | | |
| | proto-oncogene, | | |
| | bHLH | | |
| | transcription | | |
| | factor | | |
| TP63 | Also known as | NP_001108450 | NP_001120731 |
| | Tumor protein | NP_001108451 | NP_001120732 |
| | p63, | NP_001108452 | NP_001120733 |
| | transformation- | NP_001108453 | NP 001120734 |
| | related protein 63 | NP_001108454 | NP_001120735 |
| | AIS, B(p51A), | | |
| | B(p51B), EEC3, | | |
| | KET, LMS, NBP, | | |
| | OFC8, RHS, | | |
| | SHFM4, TP53CP, | | |
| | TP53L, TP73L, | | |
| | p40, p51, p53CP, | | |
| | p63, p73H, p73L, | | |
| | tumor protein p63 | | |
| ANTP63a | Also known as | NP_001108452 | NP_035771.1 |
| | the delta isoform | | |
| | of TP63 | | |

### Immunodeficiency

The invention also provides a method of treating immunodeficiency in a subject. Immunodeficiency refers to a condition or state in which one or more components of the immune response is impaired, resulting in an inability to resolve infection or disease effectively. Immunodeficiency disorders include primary immunodeficiency (PID), inherited conditions resulting from genetic mutations, and secondary immunodeficiency (SID), in which immunodeficiency is acquired as a result of disease or environmental factors (for example, chemotherapy).

In some embodiments, the method comprises administering to the subject an isolated thymic epithelial stem cell as described herein, or related cell culture compositions, pharmaceutical compositions or thymic constructs as also described herein.

In some embodiments, the invention provides a method of treating a disease or disorder in a subject, wherein the disease or disorder is a primary immunodeficiency. In some embodiments, the invention provides a method of treating a disease or disorder in a subject, wherein the disease or disorder is a secondary immunodeficiency.

### Central Tolerance Dysfunction

Central tolerance refers to the mechanism by which the T cell repertoire is selectively modified during development to remove T cells with high affinity for self-antigens. Failure or dysfunction of central tolerance can lead to the development and maintenance of T cells which bind self-antigens with high affinity and cause autoimmune disease.

In some embodiments, the invention provides a method of treating a disease or disorder in a subject, wherein the disease or disorder is a disease or disorder associated with dysfunctional central tolerance.

### Autoimmune Disease

In some embodiments, the invention provides a method of treating a disease or disorder in a subject, wherein the disease or disorder is autoimmune polyendocrinopathy candidiasis ectodermal dystrophy (APECED).

In some embodiments, the invention provides a method of treating a disease or disorder in a subject, wherein the disease or disorder is an autoimmune disease. In some embodiments, the autoimmune disease is selected from myasthenia gravis, type 1 diabetes, an autoimmune myopathy, and a connective tissue disease. In some embodiments, the autoimmune disease is myasthenia gravis. In some embodiments, the autoimmune disease is type 1 diabetes. In some embodiments, the autoimmune disease is an autoimmune myopathy. In some embodiments, the autoimmune disease is a connective tissue disease.

### Cancer

In some embodiments, the invention provides a method of treating a disease or disorder in a subject, wherein the disease or disorder is a cancer, for example a thymoma, a thymic carcinoma, a sarcoma, or a neuroendocrine tumour of the thymus. In some embodiments, the cancer is a thymoma. In some embodiments, the cancer is a thymic carcinoma. In some embodiments, the cancer is a sarcoma. In some embodiments, the cancer is a neuroendocrine tumour of the thymus.

### Thymic Atrophy

The invention also provides a method of preventing or reversing atrophy of the thymus in a subject. Thymic atrophy occurs naturally throughout life by a process of involution, in which the thymus undergoes reductions in thymic mass, loss of thymic structure and changes to thymic architecture. Thymic atrophy or involution may lead to impaired T cell development, including reduced T cell repertoire and diminished T cell output, with consequent detriment to immune responses. Thymic involution may particularly impact immune function in the aged [24]. In addition, thymic atrophy may also occur following cancer treatment or in conditions such as infection or pregnancy [24]. The invention provides a method of preventing or reversing atrophy of the thymus in a subject, comprising administering to the subject an isolated thymic epithelial stem cell as described herein, or a cell culture composition, a pharmaceutical composition, or a thymic construct as also described.

### Athymia

The invention also provides a method of treating athymia in a subject, as well as methods of treating diseases or disorders associated with or caused by athymia. Congenital athymia is a fatal condition as absence of T cell results in a severe primary immune deficiency (PID) with consequent opportunistic infections that cause death in early life. DiGeorge syndrome (deletions in chromosome 22) is the first and most frequent cause of athymia, however Foxn1 loss of function (nude phenotype, thymic epithelium agenesis) or Tbx1 mutations, have been reported in several cases. In addition, there are emerging reports of congenital immune deficiencies that may be related to thymic stroma dysfunction.

In some embodiments, the invention provides a method of treating athymia in a subject, comprising administering to the subject an isolated epithelial stem cell, a cell culture composition, a pharmaceutical composition or a thymic construct as described herein.

In some embodiments, the subject referred to in the methods described herein is a mammal. In some embodiments, the subject is a human.

The present invention also encompasses the isolated thymic epithelial stem cells for use in the methods of treatment described above and herein. The invention also encompasses the cell culture compositions comprising isolated thymic epithelial stem cells, pharmaceutical compositions, or thymic constructs for use in the methods described herein. For example, the invention also provides an isolated thymic epithelial stem cell as described herein for use in a method of treating a disease or disorder in a subject.

### Methods of Drug Screening

The invention also provides a method of drug screening using the thymic epithelial stem cells as described herein. As already described, isolated thymic epithelial stem cells are capable of self-renewal and long-term expansion in vitro, which makes them a suitable model for screening a wide range of drugs or agents, for example, drugs or agents that impact proliferation, multipotency, senescence, or differentiation.

Many studies have shown that thymus involution is reversible. Several approaches have been proposed to increase proliferation and these include IL15, IL22, KGF, IGF or factors able to target RANKL signalling1. However, these conclusions have either been based on experiments in mice, with uncertain relevance to human biology, or are inferred from clinical data with potentially confounding complexities. The thymic epithelial stem cell population (TECSP) described herein has demonstrated regenerative capacity in vitro, and has utility as a complementary and unbiased approach to reversing thymus involution. As an example, TECSP may be challenged with well characterised small molecules (i.e. those with significant target annotation [25]. The cells may be fixed at optimised time points and immunostained to reveal a range of phenotypic outputs. In some embodiments, these may include Ki67+Hoechst+ (proliferation), p16 and gH2AX (senescence), AIRE or Fzf2 (functional medulla) and FOXN1 or b5T (functional cortex). Thymic epithelial stem cells may be imaged, for example using an Opera-Phenix automated microscope platform. Image analysis may be performed with the Harmony-image package. Drug screening as described herein may include the assessment of multiple parameters leading to further analysis of particular hits, including the magnitude of any drug effect and any dose-dependent effects of the drug/small molecule.

In some embodiments, thymic epithelial stem cells may be used for screening drugs that increase proliferation or proliferation markers, where a proliferation marker is any gene or molecule expressed by a cell undergoing proliferation. In some embodiments, thymic epithelial stem cells may be used for screening drugs that decrease proliferation or proliferation markers. Proliferation markers may include PCNA, MKI67, AURKB, and/or TP63 (for example ΔNTP63α). Cell cycle analysis may also be performed by FACS using viability dyes.

In some embodiments, thymic epithelial stem cells may be used for screening drugs that increase cellular senescence or senescence markers, where a senescence marker is any gene or molecule expressed by a senescent cell. In some embodiments, thymic epithelial stem cells may be used for screening drugs that decrease cellular senescence or senescence markers. Drug screening as described herein may include screening for drugs that can activate or inhibit cellular signalling pathways such as a p16-pRB axis or a p53-p21 axis.

In some embodiments, thymic epithelial stem cells may be used for screening drugs that induce or increase the expression of functional markers, where a functional marker is any gene or molecule expressed by a cell that is associated with a particular cellular function or type. For example, a functional marker may include FOXN1, which, generally, indicates a cortical thymic epithelial cell. In some embodiments, thymic epithelial stem cells may be used for screening drugs that prevent or reduce the expression of functional markers. Other functional markers include AIRE, PSMB11, TBATA, PRSS16 and FEZF2.

In some embodiments, methods of drug screening as described herein may use an isolated thymic epithelial stem cell or related cell culture compositions, pharmaceutical compositions or thymic constructs.

### Methods of Isolating a Thymic Epithelial Stem Cell

The invention provides methods of isolating or obtaining a thymic epithelial stem cell. In some embodiments, the invention provides methods of isolating a thymic epithelial stem cell from thymus, for example, for isolating or obtaining a thymic epithelial stem cell from a sample of thymus tissue. In some embodiments, the thymus is human thymus and the thymic epithelial stem cell is a human thymic epithelial stem cell.

In some embodiments, the method comprises a step of obtaining a thymic tissue sample. Alternatively, the method may comprise providing a thymic tissue sample that has been previously obtained from a subject.

The method may comprise a step of isolating thymic epithelial cells from a thymic tissue sample, in order to obtain a thymic epithelial cell fraction.

In some embodiments, the method may include a step of dissociating the thymic tissue sample to obtain single cells. The step of dissociating the thymic tissue sample may comprise treating the tissue sample with one or more enzymes. Exemplary enzymes that may be used in the method include, for example, collagenase D, dispase II, DNAse I, and combinations thereof.

In some embodiments, the method may include a step of separating single cells from a thymic tissue sample to obtain a single cell fraction.

In some embodiments, the method may include a step of substantially depleting the single cell fraction of haematopoietic cells. In some embodiments, the method may include a step of substantially depleting the single cell fraction of CD45+ cells. In some embodiments, the method may include a step of substantially depleting the single cell fraction of red blood cells. In some embodiments, the method may include a step of substantially depleting the single cell fraction of CD235+ cells. In some embodiments, the method may include a step of substantially depleting the single cell fraction of haematopoietic cells and red blood cells. In some embodiments, the method may include a step of substantially depleting the single cell fraction of CD45+ and CD235+ cells. Depletion of CD45+ and/or CD235+ cells may be achieved by any suitable method known in the art, for example, antibody-based depletion methods, magnetic separation, immunopanning, and others.

In some embodiments, the method may include a step of isolating cells that express an epithelial cell marker from a cell fraction as described herein, in order to obtain a thymic epithelial cell fraction. In some embodiments, the method may include a step of isolating EPCAM+ cells from a cell fraction as described herein, to obtain a thymic epithelial cell fraction. Isolating EPCAM+ cells may comprise labelling or staining cells for EPCAM. Any suitable methods known in the art for labelling or staining cells may be used. Isolating EPCAM+ cells may comprise separating or sorting EPCAM labelled or stained cells by methods known in the art, for example, by FACS. In some embodiments, isolating thymic epithelial stem cells comprises sorting EPCAM^{pos} cells and EPCAM^{neg}CD205^{pos} cells.

In some embodiments, the method may include a step of labelling thymic epithelial cells for CD49F, CD90, CD24, BCAM, or combinations thereof. Any suitable methods known in the art for labelling cells may be used. In some embodiments, the method may include a step of labelling thymic epithelial cells for CD49F. In some embodiments, the method may include a step of labelling thymic epithelial cells for CD90. In some embodiments, the method may include a step of labelling thymic epithelial cells for CD24. In some embodiments, the method may include a step of labelling thymic epithelial cells for BCAM. In some embodiments, the method may include a step of labelling thymic epithelial cells for CD49F and CD90. In some embodiments, the method may include a step of labelling thymic epithelial cells for CD49F and CD24. In some embodiments, the method may include a step of labelling thymic epithelial cells for CD49F and BCAM. In some embodiments, the method may include a step of labelling thymic epithelial cells for CD90 and CD24. In some embodiments, the method may include a step of labelling thymic epithelial cells for CD90 and BCAM.

In some embodiments, the method may include a step of labelling thymic epithelial cells for CD24 and BCAM. In some embodiments, the method may include a step of labelling thymic epithelial cells for CD49F, CD90 and CD24. In some embodiments, the method may include a step of labelling thymic epithelial cells for CD49F, CD90 and BCAM. In some embodiments, the method may include a step of labelling thymic epithelial cells for CD49F, CD24 and BCAM. In some embodiments, the method may include a step of labelling thymic epithelial cells for CD90, CD24 and BCAM. In some embodiments, the method may include a step of labelling thymic epithelial cells for CD49F, CD90, CD24 and BCAM.

In some embodiments, the method may include a step of isolating thymic epithelial cells which are BCAM^{pos}, CD49F^{pos}, CD90^{pos} or CD24^{neg}, or combinations thereof, to obtain isolated thymic epithelial stem cells. In some embodiments, the method may include a step of isolating thymic epithelial cells which are BCAM^{pos}. In some embodiments, the method may include a step of isolating thymic epithelial cells which are CD49F^{pos}. In some embodiments, the method may include a step of isolating thymic epithelial cells which are CD90^{pos}. In some embodiments, the method may include a step of isolating thymic epithelial cells which are CD24^{neg}. In some embodiments, the method may include a step of isolating thymic epithelial cells which are BCAM^{pos} and CD49F^{pos}. In some embodiments, the method may include a step of isolating thymic epithelial cells which are BCAM^{pos} and CD90^{pos}. In some embodiments, the method may include a step of isolating thymic epithelial cells which are BCAM^{pos} and CD24^{neg}. In some embodiments, the method may include a step of isolating thymic epithelial cells which are CD49F^{pos} and CD90^{pos}. In some embodiments, the method may include a step of isolating thymic epithelial cells which are CD49F^{pos} and CD24^{neg}. In some embodiments, the method may include a step of isolating thymic epithelial cells which are CD90^{pos} and CD24^{neg}. In some embodiments, the method may include a step of isolating thymic epithelial cells which are BCAM^{pos}, CD49F^{pos} and CD90^{pos}. In some embodiments, the method may include a step of isolating thymic epithelial cells which are BCAM^{pos}, CD49F^{pos} and CD24^{neg}. In some embodiments, the method may include a step of isolating thymic epithelial cells which are BCAM^{pos}, CD90^{pos} and CD24^{neg}. In some embodiments, the method may include a step of isolating thymic epithelial cells which are CD49F^{pos}, CD90^{pos}, and CD24^{neg}. In some embodiments, the method may include a step of isolating thymic epithelial cells which are BCAM^{pos}, CD49F^{pos}, CD90^{pos}, and CD24^{neg}.

In some embodiments, a method for isolating a thymic epithelial stem cell from thymus comprises:
a. obtaining a thymic tissue sample, and
b. isolating BCAM^{pos}CD49F^{pos}CD90^{pos}CD24^{neg} thymic epithelial cells from the thymic tissue sample to obtain isolated thymic epithelial stem cells.

In some embodiments, a method for isolating a thymic epithelial stem cell from thymus comprises:
a. obtaining a thymic tissue sample,
b. isolating thymic epithelial cells from the thymic tissue sample to obtain a thymic epithelial cell fraction, and
c. isolating BCAM^{pos}CD49F^{pos}CD90^{pos}CD24^{neg} thymic epithelial cells from the thymic epithelial cell fraction to obtain isolated thymic epithelial stem cells.

Generally, steps of separating or isolating cells (e.g. from other cell types or other tissues) in the methods described herein may be performed by any suitable method known in the art, for example, by using magnetic beads, and/or fluorescence activated cell sorting (FACS).

### Methods of Culturing

### Methods for Culturing Thymic Epithelial Stem Cells

The invention provides methods of culturing a thymic epithelial stem cell. In some embodiments, the method comprises providing at least one isolated thymic epithelial stem cell and culturing the at least one isolated thymic epithelial stem cell under conditions suitable for maintenance and expansion of the at least one isolated thymic epithelial stem cell, where maintenance and expansion have their ordinary meaning in the art.

In some embodiments, the invention provides a method of culturing a thymic epithelial stem cell, comprising:
(a) providing at least one isolated thymic epithelial stem cell,
(b) contacting the at least one isolated thymic epithelial stem cell with at least one supporting cell, and
(c) contacting the at least one isolated thymic epithelial stem cell and the at least one supporting cell with a cell culture medium.

In some embodiments, the at least one supporting cell is a feeder cell. In some embodiments, the at least one supporting cell or feeder cell is any cell suitable to support the growth of epithelial cells in culture. In some embodiments, the at least one supporting cell or feeder cell is a fibroblast cell. In some embodiments, the at least one supporting cell or feeder cell is a mouse fibroblast cell. In some embodiments, the at least one supporting cell or feeder cell is a sub-lethally irradiated mouse fibroblast cell. In some embodiments, the at least one supporting cell or feeder cell is the sub-lethally irradiated mouse fibroblast cell 3T3-J2.

In some embodiments, the cell culture medium is any medium suitable to support the growth of epithelial cells in culture. In some embodiments, the cell culture medium is serum-free medium. In some embodiments, the cell culture medium is cFAD medium. In some embodiments, cFAD medium comprises DMEM, F-12 Nutrient Mix, Fetal Bovine Serum (FBS), antibacterial agents, hydrocortisone, cholera toxin, triiodothyronine (T3) and insulin. In some embodiments, the cell culture medium is cFAD medium, wherein the cFAD medium comprises a mixture of 3:1 DMEM 1× and F-12 Nutrient mix, 10% FBS, 1% penicillin and streptomycin (100×), hydrocortisone (0.4 μg/ml), cholera toxin (10⁻¹⁰ M), triiodothyronine (2×10⁻⁹ M) and insulin (5 μg/ml). Other suitable culture mediums are known in the art.

In some embodiments, the method of culturing isolated thymic epithelial stem cells comprises contacting the isolated thymic epithelial stem cell and/or the at least one supporting cell with a growth promoting agent. In some embodiments, the growth promoting agent is a growth factor. The cells may be contacted with growth factor once, or more than once, or repeatedly, for example, every three days. In some embodiments, the growth factor may be selected from the group consisting of human epithelial growth factor (hEGF), keratinocyte growth factor (KGF), IL-22, BMP4, RANKL or a sex steroid inhibitor. Other suitable growth promoting agents and growth factors are known in the art.

In some embodiments, the invention provides a method of culturing a thymic epithelial stem cell, comprising:
(a) providing at least one isolated thymic epithelial stem cell,
(b) contacting the at least one isolated thymic epithelial stem cell with at least one sub-lethally irradiated mouse fibroblast cell, and
(c) contacting the at least one isolated thymic epithelial stem cell and the at least one sub-lethally irradiated mouse fibroblast cell with a cFAD cell culture medium.

Suitable conditions for maintaining the thymic epithelial stem cells and/or the supporting cells in culture are known in the art. For example, cells may be maintained at a temperature between about 30° C. to about 40° C., about 35° C. to about 39° C., about 36° C. to about 38° C., or at a temperature of about 37° C.

In some embodiments, the isolated thymic epithelial stem cells may be cultured until they reach confluence or sub-confluence. In some embodiments, the thymic epithelial stem cells may be cultured (e.g. maintained in culture) for a specified number of population doublings (PD). Population doubling is the total number of times the cells in a given population have doubled during in vitro culture, as known in the art.

The isolated thymic epithelial stem cell to be cultured may be any type of thymic epithelial stem cell. In some embodiments, the isolated thymic epithelial stem cell is a cortical thymic epithelial stem cell, for example one that expresses CD205. In some embodiments, the isolated thymic epithelial stem cell is CD205^{pos}EPCAM^{neg}. In some embodiments, the isolated thymic epithelial stem cell is a medullary thymic epithelial stem cell, for example one that does not express CD205. In some embodiments, the isolated thymic epithelial stem cell is CD205^{neg}EPCAM^{pos}.

As noted elsewhere, thymic epithelial stem cells of any type (e.g. cortical or medullary) can give rise to differentiated thymic epithelial cells of both cortical and medullary lineage, regardless of whether the initial isolated thymic epithelial stem cell expressed cortical or medullary markers when isolated. It should also be understood that the protein expression of an isolated thymic epithelial stem cell may change over time and in culture. For example, an isolated thymic epithelial stem cell that expresses CD205 when first isolated from thymus tissue may lose or reduce expression of CD205 in culture. Such changes in protein or gene expression do not necessarily impact the ability of the cell to self-renew or give rise to multiple differentiated cell types, i.e. they do not generally affect stemness.

Any of the culture methods described herein may also be applied to a bulk thymic cell fraction, for example, a single cell fraction obtained following dissociation of thymus tissue, without isolating thymic epithelial stem cells from other cell types. Similarly, the culture methods described herein may also be applied to a population of thymic epithelial cells, comprising at least one thymic epithelial stem cell. In some embodiments, such a population of thymic epithelial cells may be obtained by isolating thymic cells which are EPCAM^{pos}, and/or cells which are EPCAM^{neg}/CD205^{pos}.

Any of the culture method described herein may further comprise a step of separating or isolating the thymic epithelial stem cells from other cell types, for example, from the supporting cells.

### Methods for Culturing Cortical Thymic Epithelial Cells

The invention also provides methods for culturing a cortical thymic epithelial cell derived from a thymic epithelial stem cell. The inventors have surprisingly discovered that culturing thymic epithelial stem cells under certain conditions favours differentiation of the thymic epithelial stem cells into cortical thymic epithelial cells. Culturing thymic epithelial stem cells under certain conditions may also encourage the growth of the cortical thymic epithelial cells relative to other conditions. In particular, the inventors have surprisingly found that conditions of low oxygen tension favour cortical differentiation and supports the growth of cortical thymic epithelial cells. Without wishing to be bound by theory, it is believed the lower oxygen tension in culture may recreate the lower vascular density (and therefore, lower perfusion) in the cortex of the thymus as compared to the medulla.

Accordingly, in some embodiments, the invention provides a method of culturing a cortical thymic epithelial cell derived from a thymic epithelial stem cell, comprising:
(a) providing a thymic epithelial stem cell, and
(b) culturing the thymic epithelial stem cell under conditions suitable for obtaining cortical thymic epithelial cells.

In some embodiments, the method comprises culturing the thymic epithelial stem cells at low oxygen tension. In some embodiments, the method comprises culturing the thymic epithelial stem cells at an O₂ tension of about 0.5% to about 10%. In some embodiments, the method comprises culturing the thymic epithelial stem cells at an O₂ tension of about 1% to about 9%. In some embodiments, the method comprises culturing the thymic epithelial stem cells at an O₂ tension of about 2% to about 8%. In some embodiments, the method comprises culturing the thymic epithelial stem cells at an O₂ tension of about 3% to about 7%. In some embodiments, the method comprises culturing the thymic epithelial stem cells at an O₂ tension of about 4% to about 6%. In some embodiments, the method comprises culturing the thymic epithelial stem cells at an O₂ tension of about 0.5%. In some embodiments, the method comprises culturing the thymic epithelial stem cells at an O₂ tension of about 1%. In some embodiments, the method comprises culturing the thymic epithelial stem cells at an O₂ tension of about 2%. In some embodiments, the method comprises culturing the thymic epithelial stem cells at an O₂ tension of about 3%. In some embodiments, the method comprises culturing the thymic epithelial stem cells at an O₂ tension of about 4%. In some embodiments, the method comprises culturing the thymic epithelial stem cells at an O₂ tension of about 5%. In some embodiments, the method comprises culturing the thymic epithelial stem cells at an O₂ tension of about 6%. In some embodiments, the method comprises culturing the thymic epithelial stem cells at an O₂ tension of about 7%. In some embodiments, the method comprises culturing the thymic epithelial stem cells at an O₂ tension of about 8%. In some embodiments, the method comprises culturing the thymic epithelial stem cells at an O₂ tension of about 9%. In some embodiments, the method comprises culturing the thymic epithelial stem cells at an O₂ tension of about 10%. In some embodiments, the method comprises culturing the thymic epithelial stem cells at an O₂ tension of about 11%. In some embodiments, the method comprises culturing the thymic epithelial stem cells at an O₂ tension of about 12%.

In some embodiments, the method comprises culturing the thymic epithelial stem cells at a CO₂ tension of about 0.5% to about 15%. In some embodiments, the method comprises culturing the thymic epithelial stem cells at a CO₂ tension of about 1% to about 14%. In some embodiments, the method comprises culturing the thymic epithelial stem cells at a CO₂ tension of about 2% to about 13%. In some embodiments, the method comprises culturing the thymic epithelial stem cells at a CO₂ tension of about 3% to about 12%. In some embodiments, the method comprises culturing the thymic epithelial stem cells at a CO₂ tension of about 4% to about 11%. In some embodiments, the method comprises culturing the thymic epithelial stem cells at a CO₂ tension of about 5% to about 10%. In some embodiments, the method comprises culturing the thymic epithelial stem cells at a CO₂ tension of about 6% to about 9%. In some embodiments, the method comprises culturing the thymic epithelial stem cells at a CO₂ tension of about 7% to about 8%. In some embodiments, the method comprises culturing the thymic epithelial stem cells at a CO₂ tension of about 0.5%. In some embodiments, the method comprises culturing the thymic epithelial stem cells at a CO₂ tension of about 1%. In some embodiments, the method comprises culturing the thymic epithelial stem cells at a CO₂ tension of about 2%. In some embodiments, the method comprises culturing the thymic epithelial stem cells at a CO₂ tension of about 3%. In some embodiments, the method comprises culturing the thymic epithelial stem cells at a CO₂ tension of about 4%. In some embodiments, the method comprises culturing the thymic epithelial stem cells at a CO₂ tension of about 5%. In some embodiments, the method comprises culturing the thymic epithelial stem cells at a CO₂ tension of about 6%. In some embodiments, the method comprises culturing the thymic epithelial stem cells at a CO₂ tension of about 7%. In some embodiments, the method comprises culturing the thymic epithelial stem cells at a CO₂ tension of about 8%. In some embodiments, the method comprises culturing the thymic epithelial stem cells at a CO₂ tension of about 9%. In some embodiments, the method comprises culturing the thymic epithelial stem cells at a CO₂ tension of about 10%.

In some embodiments, the method comprises culturing the thymic epithelial stem cells at a CO₂ tension of about 5% to about 10% and an O₂ tension of about 1% to about 9%. In some embodiments, the method comprises culturing the thymic epithelial stem cells at a CO₂ tension of about 5% to about 10% and an O₂ tension of about 2% to about 8%. In some embodiments, the method comprises culturing the thymic epithelial stem cells at a CO₂ tension of about 5% to about 10% and an O₂ tension of about 3% to about 7%. In some embodiments, the method comprises culturing the thymic epithelial stem cells at a CO₂ tension of about 5% to about 10% and an O₂ tension of about 4% to about 6%.

Also within the scope of the invention is a cortical thymic epithelial cell obtainable by the methods described herein.

### Methods for Culturing Medullary Thymic Epithelial Cells

The invention also provides methods for culturing a medullary thymic epithelial cell derived from a thymic epithelial stem cell. The inventors have surprisingly discovered that culturing thymic epithelial stem cells under certain conditions favours differentiation of the thymic epithelial stem cells into medullary thymic epithelial cells. Culturing thymic epithelial stem cells under certain conditions may also encourage the growth of the medullary thymic epithelial cells relative to other conditions. In particular, the inventors have surprisingly found that conditions of atmospheric oxygen tension favour medullary differentiation and supports the growth of medullary thymic epithelial cells. Without wishing to be bound by theory, it is believed the higher oxygen tension in culture may recreate the higher vascular density (and therefore, increased perfusion) in the medulla of the thymus as compared to the cortex. Notwithstanding the foregoing, it is also noted that thymic epithelial stem cells may also be cultured at low O₂ tension to provide a medullary thymic epithelial cell.

Accordingly, in some embodiments, the invention provides a method of culturing a medullary thymic epithelial cell derived from a thymic epithelial stem cell, comprising:
(a) providing a thymic epithelial stem cell, and
(b) culturing the thymic epithelial stem cell under conditions suitable for obtaining medullary thymic epithelial cells.

In some embodiments, the method comprises culturing the thymic epithelial stem cells under conditions of atmospheric oxygen tension. In some embodiments, the method comprises culturing the thymic epithelial stem cells at an O₂ tension of about 0.5% to 10%. In some embodiments, the method comprises culturing the thymic epithelial stem cells at an O₂ tension of about 10% to about 30%. In some embodiments, the method comprises culturing the thymic epithelial stem cells at an O₂ tension of about 11% to about 29%. In some embodiments, the method comprises culturing the thymic epithelial stem cells at an O₂ tension of about 12% to about 28%. In some embodiments, the method comprises culturing the thymic epithelial stem cells at an O₂ tension of about 13% to about 27%. In some embodiments, the method comprises culturing the thymic epithelial stem cells at an O₂ tension of about 14% to about 26%. In some embodiments, the method comprises culturing the thymic epithelial stem cells at an O₂ tension of about 15% to about 25%. In some embodiments, the method comprises culturing the thymic epithelial stem cells at an O₂ tension of about 16% to about 24%. In some embodiments, the method comprises culturing the thymic epithelial stem cells at an O₂ tension of about 17% to about 23%. In some embodiments, the method comprises culturing the thymic epithelial stem cells at an O₂ tension of about 18% to about 22%. In some embodiments, the method comprises culturing the thymic epithelial stem cells at an O₂ tension of about 19% to about 21%. In some embodiments, the method comprises culturing the thymic epithelial stem cells at an O₂ tension of about 10%. In some embodiments, the method comprises culturing the thymic epithelial stem cells at an O₂ tension of about 11%. In some embodiments, the method comprises culturing the thymic epithelial stem cells at an O₂ tension of about 12%. In some embodiments, the method comprises culturing the thymic epithelial stem cells at an O₂ tension of about 13%. In some embodiments, the method comprises culturing the thymic epithelial stem cells at an O₂ tension of about 14%. In some embodiments, the method comprises culturing the thymic epithelial stem cells at an O₂ tension of about 15%. In some embodiments, the method comprises culturing the thymic epithelial stem cells at an O₂ tension of about 16%. In some embodiments, the method comprises culturing the thymic epithelial stem cells at an O₂ tension of about 17%. In some embodiments, the method comprises culturing the thymic epithelial stem cells at an O₂ tension of about 18%. In some embodiments, the method comprises culturing the thymic epithelial stem cells at an O₂ tension of about 19%. In some embodiments, the method comprises culturing the thymic epithelial stem cells at an O₂ tension of about 20%. In some embodiments, the method comprises culturing the thymic epithelial stem cells at an O₂ tension of about 21%. In some embodiments, the method comprises culturing the thymic epithelial stem cells at an O₂ tension of about 22%. In some embodiments, the method comprises culturing the thymic epithelial stem cells at an O₂ tension of about 23%. In some embodiments, the method comprises culturing the thymic epithelial stem cells at an O₂ tension of about 24%. In some embodiments, the method comprises culturing the thymic epithelial stem cells at an O₂ tension of about 25%. In some embodiments, the method comprises culturing the thymic epithelial stem cells at an O₂ tension of about 26%. In some embodiments, the method comprises culturing the thymic epithelial stem cells at an O₂ tension of about 27%. In some embodiments, the method comprises culturing the thymic epithelial stem cells at an O₂ tension of about 28%. In some embodiments, the method comprises culturing the thymic epithelial stem cells at an O₂ tension of about 29%. In some embodiments, the method comprises culturing the thymic epithelial stem cells at an O₂ tension of about 30%.

In some embodiments, the method comprises culturing the thymic epithelial stem cells at a CO₂ tension of about 0.5% to about 15%. In some embodiments, the method comprises culturing the thymic epithelial stem cells at a CO₂ tension of about 1% to about 14%. In some embodiments, the method comprises culturing the thymic epithelial stem cells at a CO₂ tension of about 2% to about 13%. In some embodiments, the method comprises culturing the thymic epithelial stem cells at a CO₂ tension of about 3% to about 12%. In some embodiments, the method comprises culturing the thymic epithelial stem cells at a CO₂ tension of about 4% to about 11%. In some embodiments, the method comprises culturing the thymic epithelial stem cells at a CO₂ tension of about 5% to about 10%. In some embodiments, the method comprises culturing the thymic epithelial stem cells at a CO₂ tension of about 6% to about 9%. In some embodiments, the method comprises culturing the thymic epithelial stem cells at a CO₂ tension of about 7% to about 8%. In some embodiments, the method comprises culturing the thymic epithelial stem cells at a CO₂ tension of about 0.5%. In some embodiments, the method comprises culturing the thymic epithelial stem cells at a CO₂ tension of about 1%. In some embodiments, the method comprises culturing the thymic epithelial stem cells at a CO₂ tension of about 2%. In some embodiments, the method comprises culturing the thymic epithelial stem cells at a CO₂ tension of about 3%. In some embodiments, the method comprises culturing the thymic epithelial stem cells at a CO₂ tension of about 4%. In some embodiments, the method comprises culturing the thymic epithelial stem cells at a CO₂ tension of about 5%. In some embodiments, the method comprises culturing the thymic epithelial stem cells at a CO₂ tension of about 6%. In some embodiments, the method comprises culturing the thymic epithelial stem cells at a CO₂ tension of about 7%. In some embodiments, the method comprises culturing the thymic epithelial stem cells at a CO₂ tension of about 8%. In some embodiments, the method comprises culturing the thymic epithelial stem cells at a CO₂ tension of about 9%. In some embodiments, the method comprises culturing the thymic epithelial stem cells at a CO₂ tension of about 10%.

In some embodiments, the method comprises culturing the thymic epithelial stem cells at a CO₂ tension of about 5% to about 10% and an O₂ tension of about 10% to about 30%. In some embodiments, the method comprises culturing the thymic epithelial stem cells at a CO₂ tension of about 5% to about 10% and an O₂ tension of about 11% to about 29%. In some embodiments, the method comprises culturing the thymic epithelial stem cells at a CO₂ tension of about 5% to about 10% and an O₂ tension of about 12% to about 28%. In some embodiments, the method comprises culturing the thymic epithelial stem cells at a CO₂ tension of about 5% to about 10% and an O₂ tension of about 13% to about 27%. In some embodiments, the method comprises culturing the thymic epithelial stem cells at a CO₂ tension of about 5% to about 10% and an O₂ tension of about 14% to about 26%. In some embodiments, the method comprises culturing the thymic epithelial stem cells at a CO₂ tension of about 5% to about 10% and an O₂ tension of about 15% to about 25%. In some embodiments, the method comprises culturing the thymic epithelial stem cells at a CO₂ tension of about 5% to about 10% and an O₂ tension of about 16% to about 24%. In some embodiments, the method comprises culturing the thymic epithelial stem cells at a CO₂ tension of about 5% to about 10% and an O₂ tension of about 17% to about 23%. In some embodiments, the method comprises culturing the thymic epithelial stem cells at a CO₂ tension of about 5% to about 10% and an O₂ tension of about 18% to about 22%. In some embodiments, the method comprises culturing the thymic epithelial stem cells at a CO₂ tension of about 5% to about 10% and an O₂ tension of about 19% to about 21%. In some embodiments, the method comprises culturing the thymic epithelial stem cells at a CO₂ tension of about 5% to about 10% and an O₂ tension of about 20%.

Also within the scope of the invention is a medullary thymic epithelial cell obtainable by the methods described herein.

### Morphology

Thymic epithelial stem cells according to the invention may also be identified by their distinct cellular morphology in culture, for example by their shape, structure, form, size and organisation. The inventors have surprisingly discovered that thymic epithelial stem cells in culture can be distinguished from other cell types based on their morphology. The inventors have found that cultured (i.e. isolated cells, expanded in vitro) thymic epithelial stem cells exhibit a “refractive edges” morphology, in which the cells are highly motile instead of adhering to each other. Thymic epithelial stem cells with a refractive edges morphology may stain weakly for Rhodamine B in vitro. Thymic epithelial stem cells with a refractive edges morphology may express or co-express the markers described above (for example, TE-7, THY1, VIM, FN1, TIMP1, IFITM3).

### Multipotent

As used herein, the term “multipotent” refers to the ability of a cell, usually a stem cell, to self-renew by dividing and to develop into multiple specialised cell types. Multipotent cells are capable of giving rise to terminally differentiated cells. In the present invention, isolated thymic epithelial stem cells are generally multipotent. Isolated thymic epithelial stem cells as described herein may retain multi-lineage differentiation potency in vitro.

In particular, thymic epithelial stem cells as described herein may give rise to both cortical and medullary differentiated thymic cell types, irrespective of whether the thymic epithelial stem cell was cortical or medullary in origin. For example, an isolated cortical thymic epithelial stem cell (e.g. one expressing CD205 and/or having low levels of EPCAM expression) is generally able to give rise to both cortical and medullary differentiated cells.

### Pharmaceutical Composition

As used herein, the term “pharmaceutical composition” refers to a composition in which an active agent is formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. A pharmaceutical composition may be formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous, intratumoral, or epidural injection as a sterile solution or suspension, or sustained-release formulation; topical application, for example, as a cream, ointment, or a controlled-release patch or spray applied to skin, lungs, or oral cavity; intravaginally, intrarectally, sublingually, ocularly, transdermally, nasally, pulmonary, and to other mucosal surfaces.

In some embodiments, a pharmaceutical composition as described herein may comprise a pharmaceutically acceptable carrier, diluent or excipient. As used herein, the term “pharmaceutically acceptable” applied to the carrier, diluent, or excipient used to formulate a composition as disclosed herein means that the carrier, diluent, or excipient must be compatible with the other ingredients of the composition and not deleterious to the recipient thereof.

### Protein Expression and Surface Markers

The term “surface markers” refers to a molecule, such as a protein, that is expressed on the surface of a cell. Patterns of protein expression and the presence of particular surface markers can be used to identify and isolate thymic epithelial stem cells of the invention, and to distinguish them from other cell populations. Protein expression and cell surface markers can be detected by any suitable method. For example, protein expression can be detected by immunohistochemistry (IHC), immunocytochemistry (ICC), magnetic-activated cell sorting (MACS), fluorescence activated cell sorting (FACS), mass cytometry or proteomics.

In some embodiments, thymic epithelial stem cells express BCAM. In some embodiments, thymic epithelial stem cells express CD49F. In some embodiments, thymic epithelial stem cells express CD90. In some embodiments, thymic epithelial stem cells do not express CD24. In some embodiments, thymic epithelial stem cells express BCAM and CD49F. In some embodiments, thymic epithelial stem cells express CD49F and CD90. In some embodiments, thymic epithelial stem cells express BCAM and CD90. In some embodiments, thymic epithelial stem cells express BCAM and CD49F and do not express CD24. In some embodiments, thymic epithelial stem cells express CD49F and CD90 and do not express CD24. In some embodiments, thymic epithelial stem cells express BCAM and CD90 and do not express CD24. In some embodiments, thymic epithelial stem cells express BCAM, CD49F and CD90. In some embodiments, thymic epithelial stem cells express BCAM, CD49F and CD90 and do not express CD24.

Expression of a surface marker or level of expressed protein may be described in positive or negative terms. A cell may be negative for a particular marker when the level of expression of that marker is equivalent or comparable to that of a negative control, for example a cell that is known not to express that particular marker. For example, a cell may be described as negative for a marker when the detectable level of that marker corresponds to a fluorescence intensity signal with a distribution comparable to the one of the negative control on a fluorescence log plot. The negative control may either be an unlabelled sample (e.g. a sample that does not comprise a detectable label) or the compensation control, cells stained with all the fluorophores minus the one of the target protein, a.k.a. Fluorescence Minus One (FMO). A negative control may be used to set the PMT voltage values. A cell may be positive for a particular marker when the level of expression of that marker is increased compared to a control, e.g. a negative control. A cell may be positive for a particular marker when the level of expression of that marker is increased compared to a negative control by at least 10%, for example increased by at least 20%, increased by at least 30%, increased by at least 40%, increased by at least 50%, increased by at least 60%, increased by at least 70%, increased by at least 80%, increased by at least 90% or increased by at least 100% compared to a negative control. A cell may be positive for a particular marker when the level of expression of that marker is comparable to a positive control (i.e. a marker known to be expressed on the same cells).

The amount, degree or level of expression of a surface marker or expressed protein may also vary. For example, cells may exhibit a low level of expression of a surface marker. Cells may exhibit a high level of expression of a surface marker. Cells may exhibit an intermediate level of expression of a surface marker. The level of expression of a surface marker can be defined according to whether the surface marker is expressed to a level that is above or below a threshold value. The level of expression of a surface marker can be determined by any suitable method known in the art. For example, the level of expression of a surface marker may be determined by FACS.

In some embodiments, a cell has an intermediate level of expression of a surface marker if the cell expresses the marker at a level 1-2 logs (i.e. 10 to 100 times) higher compared to a negative control (e.g. a cell known not to express the surface marker). In some embodiments, a cell has a high level of expression of a surface marker if the cell expresses the marker at a level 1-2 logs (i.e. 10 to 100 times) higher than a cell that expresses the surface marker at an intermediate level (e.g. a cell as defined above), or 3-4 logs (i.e. 100 to 1000 times) higher compared to a negative control (e.g. a cell known not to express the surface marker). Methods of determining expression thresholds are known in the art.

In some embodiments, thymic epithelial stem cells exhibit a high level of expression of CD49F (CD49F^{high}). In some embodiments, thymic epithelial stem cells exhibit a high level of expression of CD90 (CD90^{high}). In some embodiments, thymic epithelial stem cells are BCAM^{pos} and CD49F^{high}. In some embodiments, thymic epithelial stem cells are BCAM^{pos} and CD90^{high}. In some embodiments, thymic epithelial stem cells are CD49F^{high} and CD90^{high}. In some embodiments, thymic epithelial stem cells are BCAM^{pos}, CD49F^{high}, and CD90^{high}. In some embodiments, thymic epithelial stem cells are BCAM^{pos}, CD49F^{high}, and CD24^{neg}. In some embodiments, thymic epithelial stem cells are BCAM^{pos}, CD90^{high} and CD24^{neg}. In some embodiments, thymic epithelial stem cells are CD49F^{high}, CD90^{high} and CD24^{neg}. In some embodiments, thymic epithelial stem cells are BCAM^{pos}, CD49F^{high}, CD90^{high} and CD24^{neg}.

In some embodiments, thymic epithelial stem cells express EPCAM, FN1, IFITM3, TIMP1 or combinations thereof. In some embodiments, thymic epithelial stem cells express EPCAM. In some embodiments, thymic epithelial stem cells express FN1. In some embodiments, thymic epithelial stem cells express IFITM3. In some embodiments, thymic epithelial stem cells express TIMP1. In some embodiments, thymic epithelial stem cells express FN1 and IFITM3. In some embodiments, thymic epithelial stem cells express FN1 and TIMP1. In some embodiments, thymic epithelial stem cells express IFITM3 and TIMP1. In some embodiments, thymic epithelial stem cells express FN1, IFITM3 and TIMP1.

In some embodiments, isolated thymic epithelial cells may express proteins equivalent to or encoded by the genes described herein.

### Sample

As used herein, the terms “sample” or “biological sample” (used interchangeably) typically refers to a sample obtained or derived from a biological source (e.g., a tissue or organism or cell culture) of interest, as described herein. A source of interest may be an organism, such as an animal or human. The sample may comprise biological tissue or fluid.

The methods described herein relate to isolating a thymic epithelial stem cell from thymus, in particular from a thymic tissue sample. In some embodiments, the thymus or thymic tissue sample is a human thymus or thymic tissue sample. In some embodiments, the thymus or thymic tissue sample is a non-human mammal thymus or thymic tissue sample. In some embodiments, the thymus or thymic tissue sample is a mouse thymus or thymic tissue sample.

In some embodiments, the method is conducted on a sample obtained from a subject, for example a human subject, at an earlier point in time. In other embodiments of the invention, the method may comprise a step of obtaining the sample from the subject, using any suitable method.

In some embodiments, the thymic epithelial stem cell is a human cell. In some embodiments, the thymic epithelial stem cell is from a non-human mammal. In some embodiments, the thymic epithelial stem cell is from a mouse.

### Thymus

The thymus is the central organ for the development of mature, self-tolerant T cells that recognize peptide antigens in the context of self major histocompatibility (MHC) antigens. The requirement for self MHC molecules to present antigen is termed MHC restriction. Athymic individuals do not have an organ in which to generate normal numbers of MHC restricted T cells and are therefore immune-incompetent.

The thymus is made up of immature T cells called thymocytes, as well as lining cells called epithelial cells which help the thymocytes develop. T cells that successfully develop react appropriately with MHC immune receptors of the body (called positive selection) and not against proteins of the body (called negative selection). The thymus is largest and most active during the neonatal and pre-adolescent periods. By the early teens, the thymus begins to decrease in size and activity and the tissue of the thymus is gradually replaced by fatty tissue. Nevertheless, some T cell development continues throughout adult life.

Abnormalities of the thymus can result in a decreased number of T cells and autoimmune diseases such as autoimmune polyendocrine syndrome type 1 and myasthenia gravis. These are often associated with cancer of the tissue of the thymus, called thymoma, or tissues arising from immature lymphocytes such as T cells, called lymphoma. Removal of the thymus is called thymectomy.

The thymus consists of two lobes, merged in the middle, surrounded by a capsule that extends with blood vessels into the interior. The lobes consist of an outer cortex rich with cells and an inner less dense medulla. The lobes are divided into smaller lobules 0.5-2 mm diameter, between which extrude radiating insertions from the capsule along septa.

The cortex is mainly made up of thymocytes and epithelial cells. The thymocytes, immature T cells, are supported by a network of the finely-branched epithelial reticular cells, which is continuous with a similar network in the medulla. This network forms an adventitia to the blood vessels, which enter the cortex via septa near the junction with the medulla. Other cells are also present in the thymus, including macrophages, dendritic cells, and a small amount of B cells, neutrophils and eosinophils.

In the medulla, the network of epithelial cells is coarser than in the cortex, and the lymphoid cells are relatively fewer in number. Concentric, nest-like bodies called Hassall's corpuscles (also called thymic corpuscles) are formed by aggregations of the medullary epithelial cells. These are concentric, layered whorls of epithelial cells that increase in number throughout life. They are the remains of the epithelial tubes, which grow out from the third pharyngeal pouches of the embryo to form the thymus.

### Thymic Construct

As used herein, the term “thymic construct” refers to an anatomic phenocopy of native thymus reconstructed in vitro. Generally, the isolated thymic epithelial stem cells as described herein are capable of repopulating a scaffold in order to phenocopy the unique 3D epithelial network of the thymus and produce a thymic construct. The thymic construct is functional, and may establish a functional microenvironment supporting human T cell development from lymphoid progenitors in vitro and from haematopoietic stem cells in vivo. In some embodiments the thymic construct supports mature T cell development in vivo following transplantation into humanised immunodeficient mice. In some embodiments, the thymic construct may be considered an artificial organ, in particular an artificial thymus.

In some embodiments, the invention provides a thymic construct suitable for implantation into a subject, comprising an isolated thymic epithelial stem cell or cell culture composition as described herein. Thymic constructs as described herein may be suitable for use in methods of treating a disease or disorder in a subject, for example, in methods of treating athymia or thymic atrophy.

In some embodiments, the invention provides a method of producing a thymic construct suitable for implantation into a subject, the method comprising the steps of:
(i) providing an acellular scaffold;
(ii) seeding the acellular scaffold with an isolated thymic epithelial stem cell as described herein; and
(iii) culturing the seeded scaffold to produce said construct.

In some embodiments, the acellular scaffold may be a decellularized tissue scaffold or a synthetic scaffold. Such scaffolds and methods for their production are well known in the art. For example, WO0214480 describes a number of scaffold categories: (1) non-degradable synthetic polymers; (2) degradable synthetic polymers; (3) non-human collagen gels, which are non-porous; (4) non-human collagen meshes, which are processed to a desired porosity; and (5) decellularized tissue.

An acellular scaffold typically does not comprise cells or cellular components. However, it will be appreciated that for example where a scaffold is used from a biological source, e.g. a decellularized scaffold, it is possible that some cells may remain on the scaffold e.g. after decellularization, as discussed below.

In one embodiment the scaffold is an artificial scaffold, which may be a synthetic or natural polymer scaffold.

Other synthetic scaffolds may be proteinaceous in nature e.g. primarily consist of purified proteins such as collagen. Non-synthetic scaffolds may also be proteinaceous in nature, or primarily consist of a collagenous extracellular matrix (ECM) from tissue. The scaffold may be a 3-D printed scaffold, which may comprise any of the aforesaid materials.

Preferably the scaffold will be a decellularized (biological) matrix.

In some embodiments the scaffold comprises an acellular thymus scaffold.

In preferred embodiments the acellular thymus scaffold is an acellular whole thymus scaffold, preferably a whole thymus which has been decellularized; and in preferred embodiments the resultant construct is reconstructed thymus.

In some embodiments, the step of seeding the acellular scaffold with an isolated thymic epithelial stem cell includes seeding the acellular scaffold with a population of isolated thymic epithelial cells. In some embodiments, the population of isolated thymic epithelial cells comprises thymic epithelial stem cells. In some embodiments, the isolated thymic epithelial cells for seeding onto the acellular scaffold are substantially all thymic epithelial stem cells.

In some embodiments, the acellular scaffold is seeded with other thymic cells in addition to the thymic epithelial stem cells. Other suitable thymic cells include thymic interstitial cells, endothelial cells, fibroblasts, haematopoietic progenitors, mesenchymal cells and haematopoietic stromal cells such as dendritic cells.

The method of producing a thymic construct is generally carried out in vitro, though it will be appreciated that further cell proliferation and/or differentiation and generation of the construct can occur after implantation in vivo. Thus, preferably the production of the construct is carried out in vitro until a construct is generated which is sufficiently populated with thymic epithelial cells that are sufficiently differentiated to allow successful implantation into a subject.

Further cellular proliferation in and/or on the scaffold can then subsequently occur e.g. after implantation. It will be appreciated therefore that a scaffold need not be entirely populated with the seeded cells to be useful for implantation into a subject. For example, it is possible that the scaffold has areas where seeded cells are not present, for example the scaffold may have seeded cells across at least 70%, 80%, 90%, 95% or at least 99% of its surface.

The cells used in the present methods will typically be autologous i.e. originate from or are derived from the intended recipient of the tissue or organ construct generated by the method of the invention. However, cells for use in the method may also be allogeneic, i.e. obtained or derived from a subject who is not the recipient of the tissue or organ construct to be generated. Further, xenogeneic cells may be used, i.e. cells derived from a different species to the recipient of the tissue/organ construct. The cells may also be produced from pluripotent stem cells or induced pluripotent stem cells.

Aspects and embodiments described herein with the term “comprising” may include other features or steps within the scope. It is also understood that aspects and embodiments described as “comprising” also describes aspect and embodiments wherein the term “comprising” is replaced by the term “consisting essentially of” or “consisting of”.

The phrase “selected from the group comprising” may be substituted with the phrase “selected from the group consisting of” and vice versa, wherever they occur herein.

It is also understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

The invention will now be further described by way of the following Examples, which are meant to serve to assist one of ordinary skill in the art in carrying out the invention and are not intended in any way to limit the scope of the invention, with reference to the Figures.

### EXAMPLES

### Materials and Methods

### Human Tissues

Postnatal thymi were donated by patients (age 3 days to 82 years old). Written informed consent has been obtained from the patient's parents or legally authorised representatives under ethical approval. Human foetal liver was provided by the Joint MRC/Wellcome Trust Human Developmental Biology Resource (HDBR) under informed ethical consent with Research Tissue Bank ethical approval.

### Thymic Epithelial Cell (TEC) and Thymic Epithelial Stem Cell (TESC) Isolation

Thymic tissue fragments were dissociated to single cells with enzymatic treatment (0.4 mg/mL Collagenase D (Roche), 0.6 mg/mL Dispase II (Roche), 40 μg/mL DNAse I (Roche)) for around 30-45 minutes, using the Gentle MACS machine (Miltenyi). After the dissociation, the supernatant was collected, filtered via cell-strainer (100 μm), centrifuged at 1200 r.p.m. for 5 minutes and cells counted with trypan blue (SIGMA-ALDRICH) to assess viability. One portion of total dissociated cell suspension was used for culture (see chapter below) and the other depleted for CD45+ and CD235+ cells by staining them with biotinylated antibodies, then incubated with magnetic negative beads (Magnisort SAV Negative Beads, Invitrogen) and ending into a magnet (STEMCELL Technologies) for 10 min. The flowthrough fraction was collected and re-passed at least 3× times through the magnet. The final enriched fraction (CD45−CD235−) was stained for surface markers to isolate epithelial cells EpCAM, CD205 and in addition for CD49F, CD90, CD24 and BCAM. Cells were sorted using FACS Aria III machine (BD) and sorted events plated in culture or were lysated in RTL for transcriptomic analysis or processed for 10-× single cell sequencing.

### Thymic Epithelial Cell (TEC) and Thymic Epithelial Stem Cell (TESC) Culture

The thymic epithelial cells, derived from the dissociation and/or sorting, were plated on a layer of sub-lethally irradiated mouse fibroblast (3T3-J2) as described previously (J. G. Rheinwald & H. Green (1975), Serial cultivation of strains of human epidermal keratinocytes: the formation of keratinizing colonies from single cells, Cell, 1975 November; 6(3).331-43). These cells were kept in culture with cFAD medium composed by a mixture of 3:1 of DMEM1× (Gibco) and F-12 Nut Mix (Gibco), 10% Fetal Bovine Serum (SIGMA-ALDRICH and Gibco), 1% penicillin and streptomycin (100×, Sigma), Hydrocortisone (0.4 μg/ml, Calbiochem), Cholera Toxin (10⁻¹⁰ M, Sigma), Triodothyronine (T3) (2×10⁻⁹ M Sigma) and Insulin (5 μg/ml, SIGMA-ALDRICH). All the reagents were filtered through a 0.22 μm strainer. Thymic epithelial cultures total dissociated and sorted EpCAM+ were kept in incubator at 37° C. in a 6% CO₂ and 20% O₂ atmosphere, while CD205^{pos}EpCAM^{neg} fraction was grown at 37° C. in a 6% CO₂ and 5% 02 atmosphere Human epithelial growth factor (hEGF, 10 ng/ml, PeproTech) was added to the cultures after three days of culture and then every other day. The thymic epithelial cells were plated 2000-6000 cells/cm². Once subconfluent, epithelial cells were harvested using TrypLe express (Gibco) for 3-5 minutes at 37 C, blocked with medium, spun down 1200 rpm and counted.

### Colony Forming Efficiency Assay

The colony forming efficiency assay or plating efficiency was performed every other passage. A specific number of cells (e.g. 300-500 cells for Bulk cultures (total dissociated); 500-1500 for sorted cells) were plated by a serial dilution in MW6 or 60 mm dishes previously plated with lethally irradiated 3T3-J2 cells. At day 4 and 8, the culture was supplemented with hEGF (10 ng/ml, PeproTech). After 12 days of culture, cells were fixed by 4% Paraformaldehyde (PFA, SIGMA-ALDRICH a) for 10 minutes and stained with Rhodamine B (1%, SIGMA-ALDRICH) for 15 min. The dish was washed with tap water and left to dry at room temperature.

Single cells cloning has been performed as follow: thymic epithelial cells have been trypsinised and counted as described above. Once obtained the single-cell suspension, serial dilutions have been made to plate one single cell in each well into a 48-well plate, pre-coated with a layer of sub-lethally irradiated mouse fibroblast (3T3-J2).

### Coverslips Immunophenotypic Analysis

TEC were seeded at the density of 1200-2500 cells/cm2 per each well (12 wells plate) onto glass coverslips pre-seeded with irradiated 3T3-J2 and cultured up to 7 days. Then, cultured cells were fixed by 4% Paraformaldehyde (PFA, SIGMA-ALDRICH) for 10 minutes and washed twice with PBS and kept at 4° C. until the immunofluorescence (IF) staining. Quality control of primary cell cultures included Mycoplasma PCR screening, STR authentication to confirm unique profile and KarioStat™ Array (ThermoFisher, cat #905403) to screen for possible chromosomal abnormalities.

### Thymic Epithelial Cell Differentiation Assay

After harvesting, thymic epithelial cells have been plated onto the membrane of the thincert cell culture insert for well plates (Greiner Bio) with a density of 400-800 cells/mm². TEC on the membrane have been cultured in cFAD until day 2, when they have been exposed to the air, maintaining the media only below the insert for the rest of the culture. Cells have been differentiated in either Pneumocult Maintenance Medium (Stem cells technologies) or in Neurocult Base (Gibco) supplemented with B-27™ Supplement (50×, serum free Gibco), N-2 Supplement (100×, Gibco); DAPT (Gamma γ-secretase inhibitor) 10 uM has been added during the differentiation phase. From day 2 onward the media has been changed every other day. Differentiation has been stopped at several time points up to 30 days for immunofluorescence and transcriptomic analysis (qPCR). For IF, the membrane has been washed with PBS and then fixed with 4% PFA (SIGMA-ALDRICH) for 10 min. After fixation, the membrane has been washed twice in PBS and stored at 4° C. or used immediately for immunostaining. Alternatively, the membrane has been covered and washed with the BL-buffer (Promega) for the collection of the RNA and stored at −80 C up to extraction.

### Flow Cytometry Analysis

Single-cell suspensions were stained with ad hoc antibody mix in Hanks Balanced Salt Solution (HBSS, Life Technologies) supplemented with 2% FBS (Life Technologies) for 30 min on ice. DAPI (SIGMA-ALDRICH) or Zombie Live-Dead dye (Invitrogen) was used to discriminate live from dead cells. FACS phenotypic analysis was performed using Fortessa X-20 machine (BD Bioscience) and FlowJo™ software.

In Vivo Assay: Grafting Thymic Rat Scaffolds into NSG-Nude Mice

All animal procedures were in accordance with ethical approval and UK Home Office Project License (PPL) PP9619702 NOD.Cg-Prkdcscid.Il2Rγctm1WjI (NSG) and NOD.CgFoxn1em1Dvs. Prkdcscid.Il2Rγctm1WjI (NSG-Nude, Stock No: 026263) were obtained from Jackson Laboratory, re-derived and maintained at The Francis Crick Institute's biological resource facility.

CD and Winster ham rats were purchased from Charles River Laboratories. Rat thymi vascular microsurgery, perfusion and decellularization has been performed as described in [44].

Foetal Liver (FL) CD34+ were isolated from 18 weeks post conception (wpc) human foetal liver samples. Tissue digestion was performed at 37° C. using an enzymatic solution (0.1 U/mL Collagenase A (Roche), 0.8 U/mL Dispase II (Gibco) and 100 μg/ml DNase I (Roche) in RPMI, 2% FBS and 1% Penicillin/Streptomycin). Cells were pelleted and processed for ammonium chloride red cell lysis. FL mononuclear cells (MNCs) were magnetically sorted for CD34 positivity using EasySep™ Human CD34 Positive Selection Kit II, according to manufacturer's instructions (STEMCELL Technologies).

NSG-nude and NSG (8 to 12-week-old) mice were sub-lethally irradiated with 2.25 Gy from a 137Caesium source (IBL 637 Gamma Irradiator). For primary engraftment, 200k of purified foetal liver (18wpc) CD34+ cells were injected intravenously per mouse. Engraftment of human cells in the murine bone marrow was assessed at sacrifice. Scaffold repopulation was achieved as follows: cell suspension in cFAD medium of TEC and thymic interstitial cells (TIC) were injected (2M cells, ratio 5:1 respectively in 100 μl per lobe) using Insulin Syringes (Terumo, 29.5 G) into decellularized rat thymi scaffolds and cultured for five days in cFAD. huFoetal liver CD34+(18wpc) (230,000)±VeraVec (100,000) cells were injected the day before implant (day 6) in 50 ul volume of co-culture medium (DMEM 1× (Gibco), 10% FBS (SIGMA-ALDRICH), 1% penicillin and streptomycin (100×, SIGMA-ALDRICH), Triodothyronine (T3) (2×10-9 M SIGMA-ALDRICH), Insulin (5 μg/ml, SIGMA-ALDRICH) and Cytokines (Interleukin-7, 5 ng/mL (PeproTech), Stem Cell Factor 5 ng/mL (PeproTech) and FLT3-L (5 ng/mL, PeproTech)) Sub-cutaneous implantation of the scaffold was performed in NSG and NSG-Nude mice 4 to 5 weeks post-CD34+ injection as previously described. Mice were culled at 10 and 16 wpt week post-transplant (wpt). We carried out subcutaneous transplantation in three humanized NSG-Nude and three humanized NSG mice: all mice showed bone marrow reconstitution. The mice were implanted each with 4 repopulated scaffolds for a total of 24 repopulated scaffolds, 20 of which were retrieved.

### RNA Isolation and RT-qPCR

Cultured cells were collected for gene expression analysis in either BL+TG-Buffer from ReliaPrep™ kit (Promega) or in Trizol TRI Reagent (SIGMA-ALDRICH) following the manufacturer's instructions. Precipitated and dried RNA was re-suspended in nuclease free water (Qiagen). RNA concentration was measured using Nanodrop1000 (ThermoScientific). RNA was converted into cDNA with GoScript™ Reverse Transcriptase kit (Promega) according to the manufacturer's protocol. cDNA concentration was adjusted to 10 ng/μl. Quantitative (q)PCR was performed using PCR master mix (PrecisionPLUS-R—Primerdesign Ltd) with low-ROX and Taqman qPCR probes (Integrated DNA Technology in MicroAmp Fast Optical 96 well Reaction Plates (Applied Biosystems) using the QuantStudio 3 Real-Time PCR System (Applied Biosystems).

### Single-Cell RNA Sequencing—10× Genomics of Thymic Fresh Tissue and Cultivated Cells

Trypsinized cells and FACS-sorted events were resuspended in final volume of 50 μl of HBSS+0.04% BSA solution.

Cell numbers were confirmed using an Eve automated cell counter (NanoEnTek). Where possible an appropriate volume for 10,000 cells was adjusted with nuclease-free water. Reverse transcription and library construction were prepared by following Chromium single-cell 3′ reagent v3 protocol (10× Genomics) according to the manufacturer's recommendations. Total complementary-DNA synthesis was performed using 12 amplification cycles, with final cDNA yields ranging from ˜3 ng/μl to 15 ng/μl. The 10× Genomics sequencing libraries were constructed as described and sequenced on an Illumina HiSeq 4000.

### Bioinformatics Data Analysis of Single-Cell Data

10× FASTQ-files were aligned with the CellRanger toolkit (10× Genomics, version 5.0.0) toolkit to the Ensembl human GRCh38 reference transcriptome. To identify mouse feeder cells in the in vitro experiment, the in vitro dataset was also aligned to the combined human GRCh38 and mouse mm10 reference transcriptome. Mouse feeder cells were filtered from the overall cell population.

Individual samples were assessed using the Seurat R-package (version 4.0.5) (Stuart et al, 2019) and filtered based on the following percentages of mitochondrial genes: In vivo samples: Epip6<20%; Epip7<20%; Epip10<30%; Epip14<30%; all in vitro culture samples were filtered at <20%; Filtering on the Seurat nFeature RNA parameter was done at nFeature RNA>200 for all in vivo samples and at >750 for all in vitro samples. Individual samples in the in vitro and in vivo experiment were integrated using the canonical correlation analysis method of the Seurat R-package.

For in vivo datasets, we profiled 3872 cells for sorted EpCAM^{low} CD205^{pos} (cortex) and 4935 cells for sorted EpCAM^{high} CD205^{neg} (medulla); 1349 cells for cTEC CD49f^{pos} and 1414 for mTEC CD49f^{pos}.

For in vitro datasets, we profiled 2798 and 1871 cells for sorted cTEC-Polykeratin; 3463 and 3430 cells for sorted mTEC-Polykeratin; 3463 and 3430 cells for bulk TEC cultures; and 5509 cells for skin keratinocytes cultures after performing QC and mouse feeder layer reads removal. For second in vitro dataset, two additional replicates of thymic epithelial cells (5968 and 3876 cells), one of skin keratinocytes (7633 cells) and one of basal airways cells (4237 cells) have been profiled after performing QC and mouse feeder layer reads removal

### Differential Gene Expression and Trajectory Analyses

Differential gene expression analyses between various single-cell populations were carried out using the glmGamPoi R-package version 1.2.0. (Ahlmann-Eltze and Huber, 2021). Unbiased trajectory analyses were carried out using the monocle3 R-package version 0.2.2. Marker-gene directed trajectory analyses were carried out using the Ouija R-package version 0.99.1 [40].

Gene Expression Profile nCounter Analysis

The multiplexed NanoString nCounter™ Stem Cells panel enriched with a bespoke Stem Plus panel was used as expression assay for profiling of 780+25 (thymic) human genes (NanoString Technologies, Inc., Seattle, WA, USA). The assay was performed according to manufacturer's protocol. In brief, crude cell lysate was used as input material and sorted TEC (CD205^{pos} BCAM^{pos}, CD205^{neg} BCAM^{neg}, EpCAM^{pos} BCAM^{pos} and EpCAM^{pos} BCAM^{neg}) were lysed in RLT lysis buffer (Qiagen) at 2.000-10.000 cells/sample.

Samples were snap-frozen on liquid nitrogen and stored at −80° C. mRNA expression was measured on NanoString nCounter™ MAX system in a final volume of 15 μl by using 2 μl cell lysates mixed with a 3′ biotinylated Capture Probe/Capture probe+ and a 5′ Reporter Probe/Reporter probe+ tagged with a fluorescent barcode. Probes and target transcripts were hybridized overnight at 65° C. for 22 hours following manufacturer recommendations. Data were collected on an nCounter digital analyzer (NanoString™), and imported into nSolver Analysis Software v4.0 (www.nanostring.com) for data quality check, background thresholding and normalization. The quality of the run for each sample was confirmed by the quality control that considered the 6 spiked-in RNA Positive Control and the 8 Negative controls present in the panel, the FOV (fields of view per sample) counted and the binding density.

Gene expression data were normalized in two steps: (a) by using all the 12 housekeeping genes present in the panels and (b) by adjusting the number of cells/sample to 2000 cells total for each sample. Background level was determined by mean counts of 8 negative control probes plus two standard deviations. Samples that contain less than 50% of probes above background, or that have imaging or positive control linearity flags, were excluded from further analysis. Probes that have raw counts below background in all samples were excluded from differential expression analysis to avoid false positive results. For the differential gene expression analysis, we followed the procedure lined out previously [26]. NanoString count matrices were normalized using the RUVSeq Rpackage and differential gene expression was performed using the R-package DESeq2

### Histology

Human thymic samples were fixed (for 2 hrs to overnight) in 4% PFA and processed for either cryo- or paraffin-embedding. For cryo-embedding, fixed tissue was equilibrated in sucrose 25% and embedded in O.C.T. compound (VWR). Cryosections (thickness, 7 μm) were cut on a Leica Cryostat 3050. For paraffin-embedding, a Leica PelorisII tissue processor and Sakura Tissue-Tech embedding station were used. Paraffin section (thickness, 3-5 μm) were produced using ThermoFisher rotary microtome.

Cryo- or Paraffin sections were stained with haematoxylin-eosin using an automatic station (Tissue-Tek Prisma) to verify histology of each tissue and subsequently used for immunohistochemistry analysis.

### Immunostaining

OCT embedded tissue sections or coverslips fixed in 4% PFA were directly blocked and permeabilised simultaneously using a solution of 5% Normal Donkey Serum (NDS, Jackson 19 Immuno Research) in PBS, containing 0.5% of TrtonX (TritonTMX-100, SIGMAALDRICH). Paraffin-embedded samples underwent heat inactivated antigen retrieval process in Cytrate Buffer (Sigma-Aldrich) pH 6.0 prior to blocking. Tissue sections/coverslips were incubated with primary antibodies 5% NDS, 0.01% TritonTMX solution overnight at 4° C. Secondary antibodies were incubated at room temperature (RT) for 45 minutes. Nuclei were counterstained with Hoechst 33432 (10-6 M) or DAPI present in the Fluoroshield™ Mounting Medium (Abcam).

### Example 1—Identification and Characterisation of Thymic Epithelial ‘Polykeratin’ Cell Cluster

Single cell RNA-sequencing (scRNA-seq) allows to define cellular heterogeneity of an organ microenvironment. Yet, full characterization of thymic stroma is affected by several factors, including the limiting number of epithelial cells (<0.02%) that can be efficiently isolated from an organ where the vast majority (>99%) of the cellularity is represented by developing thymocytes [27,28,29,44].

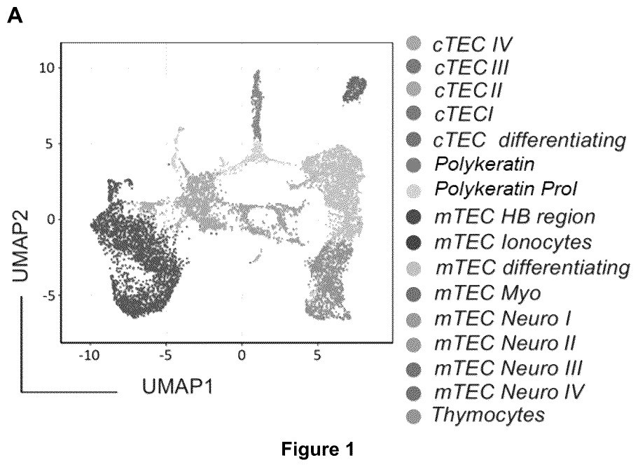

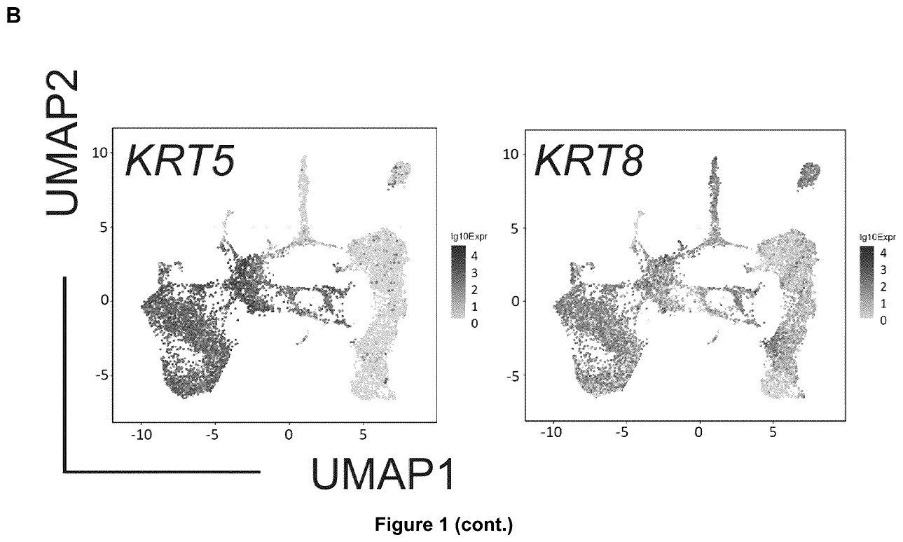

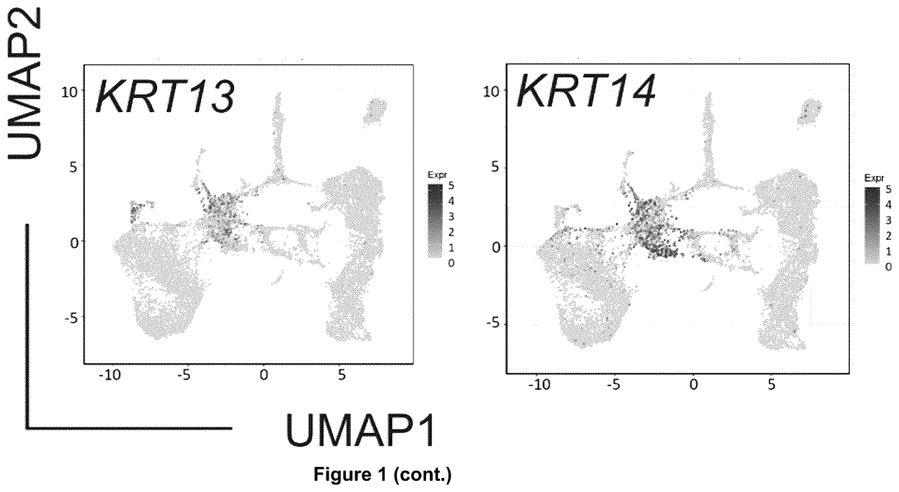

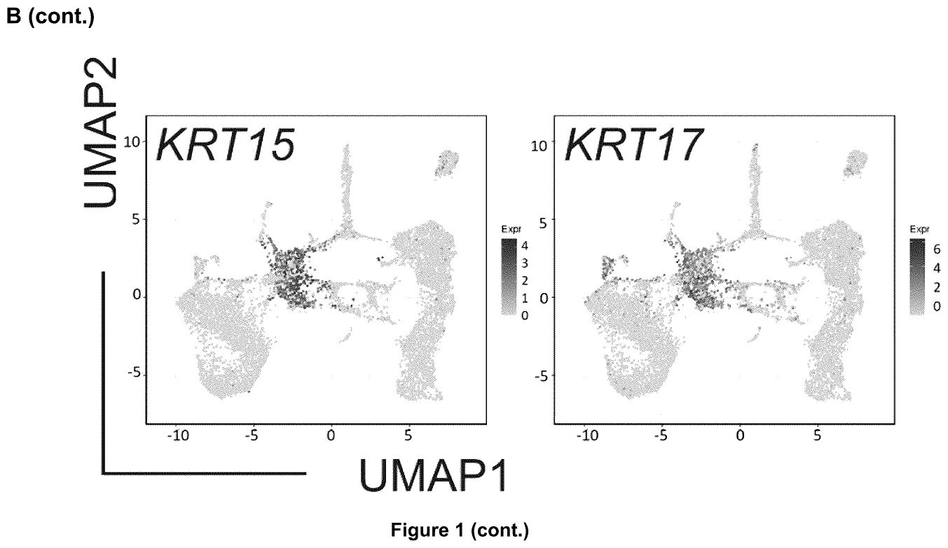

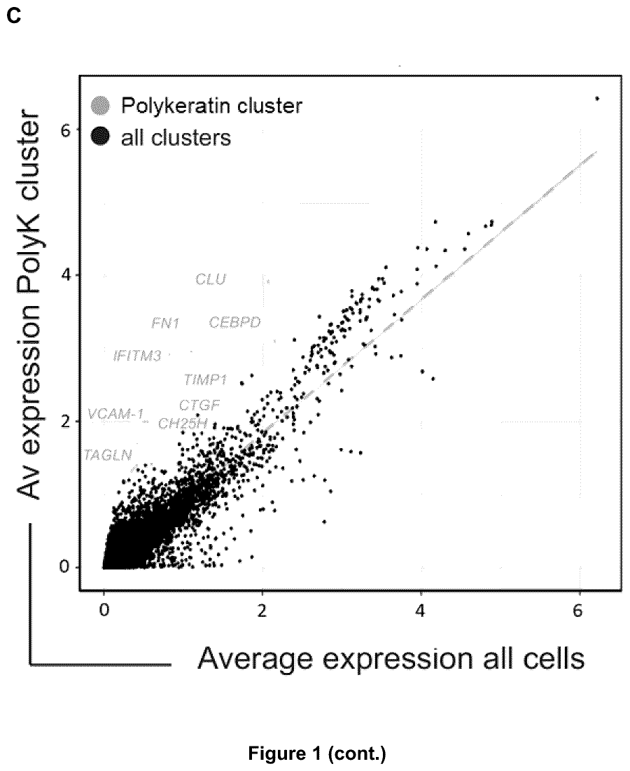

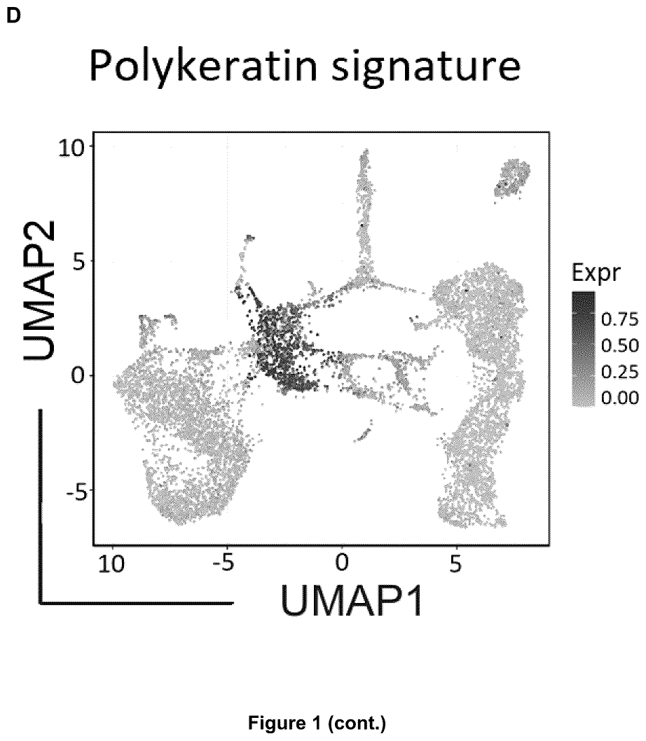

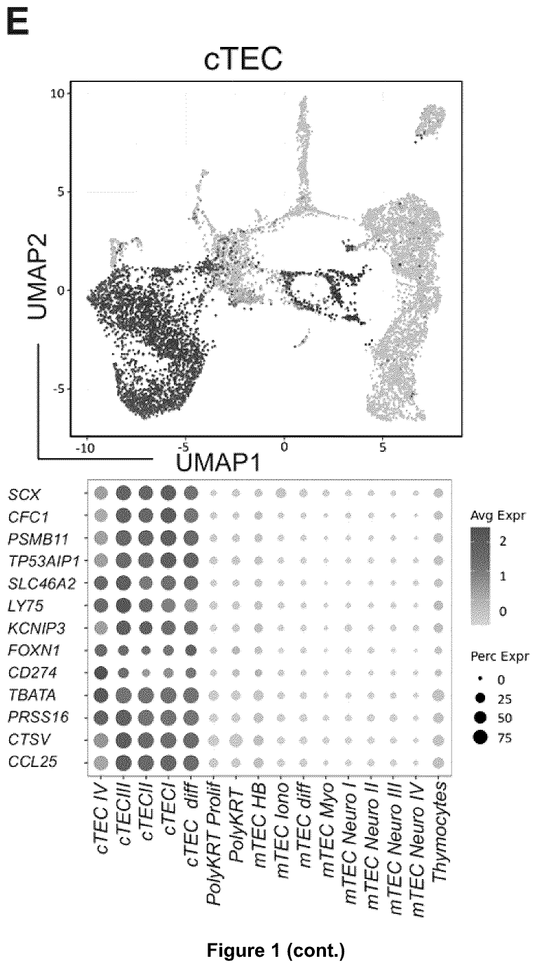

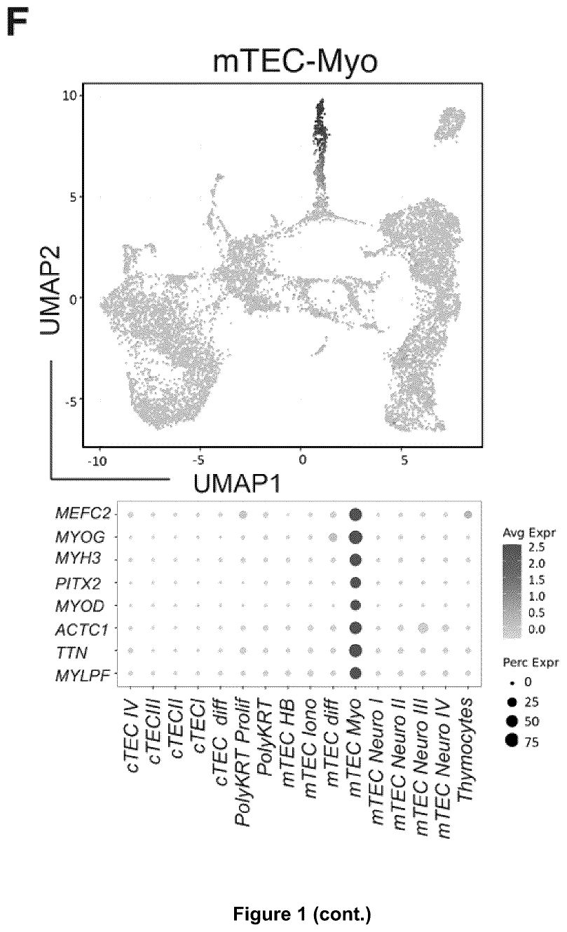

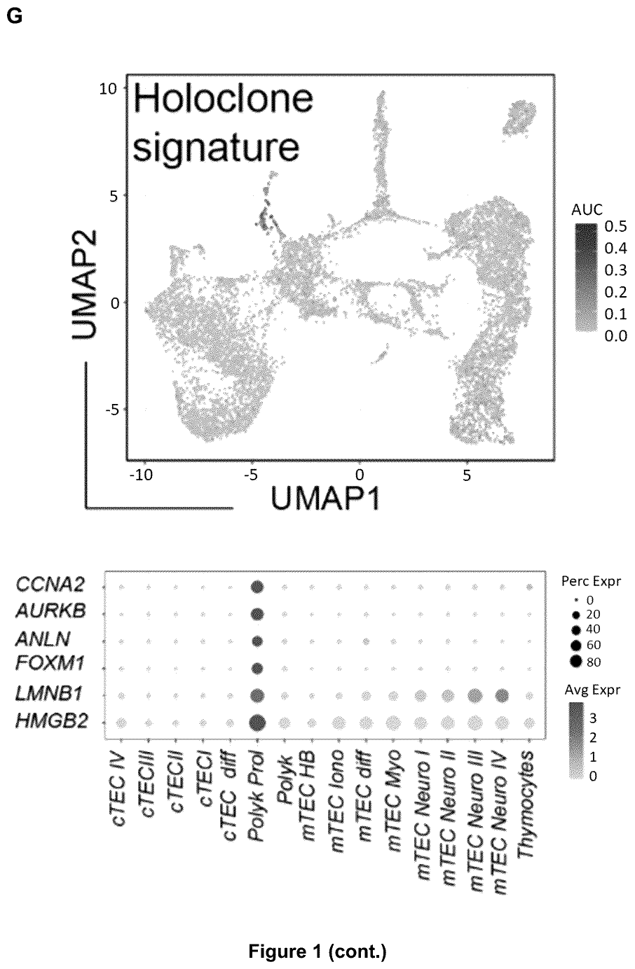

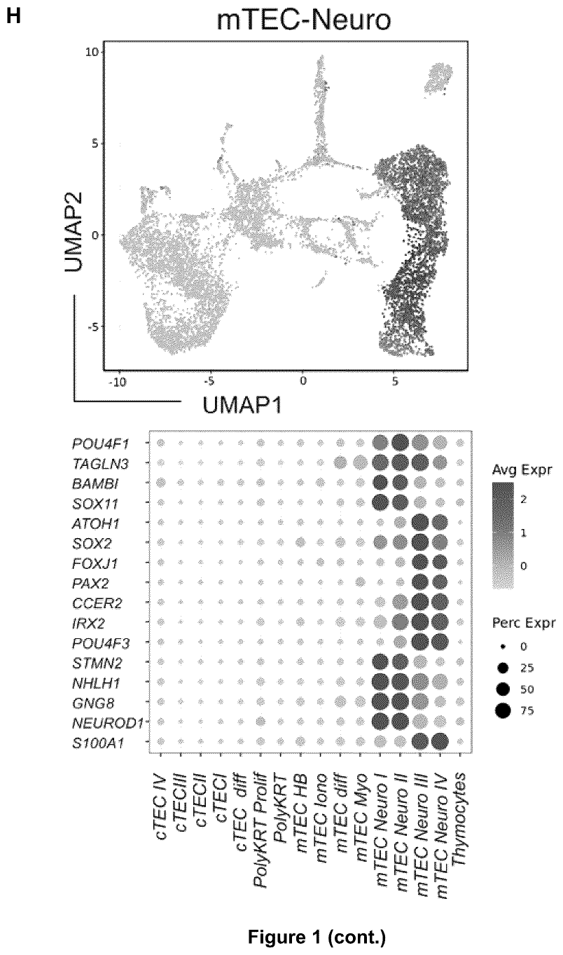

To identify at high resolution all epithelial cells of the postnatal thymus, we performed independent scRNA-sequencing analysis of cortical and medullary populations sorted based on EpCAM^{neg}CD205^{pos}(cortex) and EpCAM^{pos}CD205^{neg} (medulla) after several rounds of stromal cell enrichment. All thymic epithelial cells (TEC) were visualised in a (Uniform Manifold Approximation and Projection) UMAP plot where cortical cell (cTEC) clusters are in light/dark green colours and medullary cell (mTEC) clusters in pink/red (FIG. 1A). Independent scRNA-seq of each of the cTEC and mTEC sorted populations allowed us to define a specific cluster common to cortex and medulla that we named ‘Polykeratin’ and is visualized in yellow/orange in the UMAP plot (FIG. 1A). In this cluster, cells expressed multiple cytokeratins (KRTs) which are the intermediate filament proteins that under physiological conditions define the specific lineage differentiation of simple, stratified, or glandular epithelial cell types in different tissues. Keratins expressed included KRT5, KRT8, KRT13, KRT14, KRT15, KRT17, KRT18, and KRT19 (FIG. 1B). Of note, Polykeratin cells co-expressed KRTs that in other tissues are associated with either proliferating SC (e.g. KRT15) or differentiated layers (e.g. 15 KRT13) (FIG. 1B). They also co-expressed KRTs that are usually found only in simple (e.g. KRT8/18) or stratified (e.g. KRT5/14) epithelia and that in the mature thymus define cTEC and mTEC respectively. Nonetheless, some KRTs that characterize other differentiated cell types, e.g. KRT7 (lung ionocytes) and KRT1/KRT10 (upper layers of epidermis), were expressed only by thymic specialized clusters, i.e. Ionocytes (Io) and cornified Hassall's Body region (HB) (FIG. 1A). Thus, the KRT profile of Polykeratin cells was broad but not wholly promiscuous.

We further studied the transcriptional profile of this cluster and defined, in addition to KRTs, a signature atypical for epithelial cells that is visualised in the average gene expression scatter plot and in the feature view UMAP (FIG. 1C, D). Of note, transcripts of several genes encoding for extracellular matrix (ECM) proteins or molecules for anchoring to ECM (including FN1, TIMP1, VCAM1) argued that Polykeratin cells could produce components of their own niche. In addition, we noted expression of genes associated with inflammation and more recently also to stemness such as CLU, CEBPD and IFITM3 (FIG. 1C, D) [30,31,32].

Another small cluster common to cortex and medulla was characterized by genes related to active proliferation, besides poly-KRT (FIG. 1A). We hypothesized that this might represent an ‘activated’ status of Polykeratin cells and that Polykeratin and Polykeratin-proliferating cells might represent the putative stem/progenitors of the postnatal thymus. Clonogenicity is a concept established for epidermal keratinocytes where the ‘holoclone’ represents the activated/proliferating, long-term expanding stem cell [6]. Given that a‘holoclone’ signature (containing CCNA2, AURKB, FOXM1, ANLN, LMNB1, HMGB2) was recently reported [15], we asked whether it was expressed by Polykeratin thymic cells. Indeed, this signature was detected in the Polykeratin-proliferating cluster, consistent with these cells represent the activated/proliferating thymic stem cells in vivo (FIG. 1G).

### Example 2—Cortical and Medullary Cell Heterogeneity Defines New Functional Clusters

As a first step to pursuing this hypothesis, we analysed in detail all other clusters of both cortex and medulla to assign their identity and gene signatures. We found four cortical clusters (cTEC-1, cTEC-II, cTEC-III and cTEC-IV) which were clearly identified by a differentiated cortical cell signature (e.g. TBATA, PRSS16, CD74, CTSV, KCNIP3) as shown in the feature view UMAP and dot plots (FIG. 1E). cTEC-I-III confirmed cortical clusters previously described by us and others [44,27,28], whereas identification of the fourth cTEC-IV cluster reflected the higher level of resolution of this study. When we determined marker genes for cTEC-IV, we observed that these differentiated cTEC expressed the signature of recently activated neurons (i.e. EGR1, ARC, JUN, FOS, ATF3) that develop synaptic plasticity [33]. This signature may reflect a specific functional status of cortical cells which express also CD274, a check in addition to the conventional cortical functional genes FOXN1, TBATA and CD205 (FIG. 1E) [34]. CD274 (PD-L1) is reported to be broadly upregulated in thymic neoplasms [35,36]. Its expression in a specialized sub-population of healthy cTEC highlights PD-L1 role in controlling activation of immature thymocytes undergoing positive selection.

In the medulla, we defined the signatures of seven differentiated and specialized cells that we grouped into five categories: myoid cells (mTEC-Myo), a progressively maturing population with molecules and transcription factors of smooth, skeletal and cardiac cells (FIG. 1F); two main groups of neuroendocrine cells (mTEC-Neurol/II), characterised by expression of GKAP1, HIGD1B, NEUROD1, NHLH1, NLRP1, SOX11, STMN2; and mTEC-NeuroIII/IV characterized by expression of IRX2, ATOH1, SOX2, PAX2, POU4F3, CCER2, S100A1 (FIG. 1H). These clusters represent two main polyendocrine cell types with novel signatures that define at single cell level the endocrine compartment of the thymus gland.

Ionocytes were defined, among others, by CFTR, FOXI1, KRT7, reflecting the CFTR-expressing pulmonary ionocyte population [37]. Finally, we defined HB-cluster cells characterized by the expression of AIRE, FEZF2, KRT1, KRT6A, KRT10 crucial for tolerance induction.

In addition to the Polykeratin, cortical and medullary specialized clusters, we identified ‘transition’ clusters that in the UMAP plot stemmed out from the Polykeratin toward either medullary or cortical differentiated cells (FIG. 1A). We asked whether these novel clusters may drive differentiation toward some mature fates and therefore would share a pre-medullary and pre-cortical signature respectively.

### Example 3—Multilineage Differentiation of Thymic Epithelial Polykeratin Cells

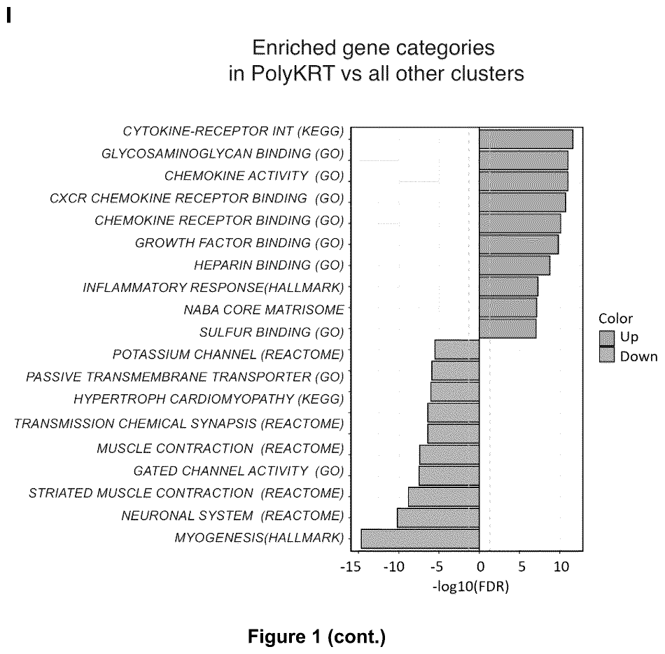

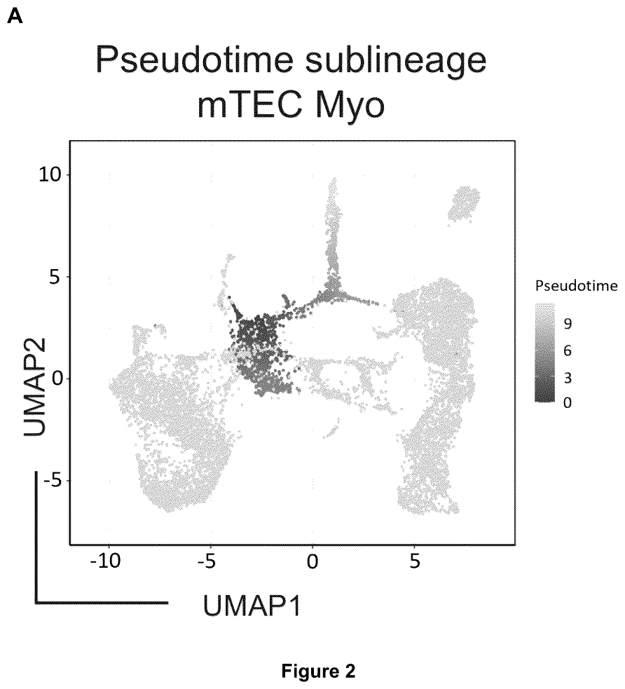

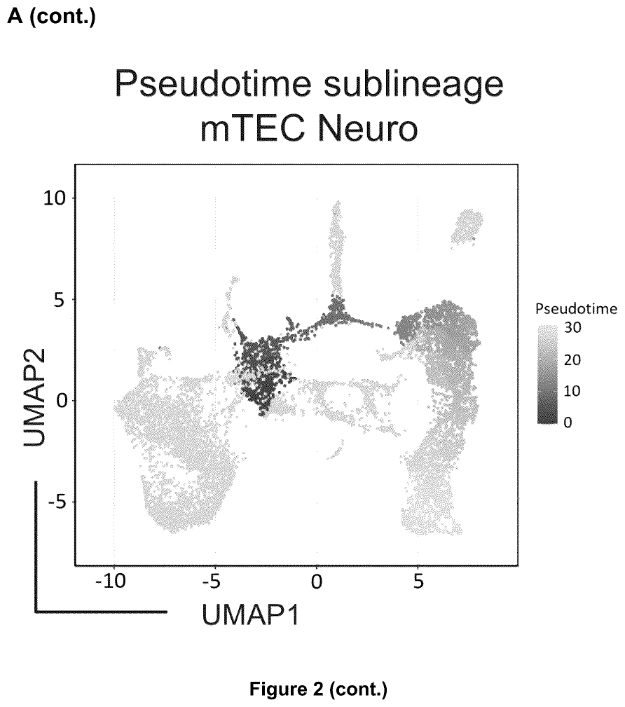

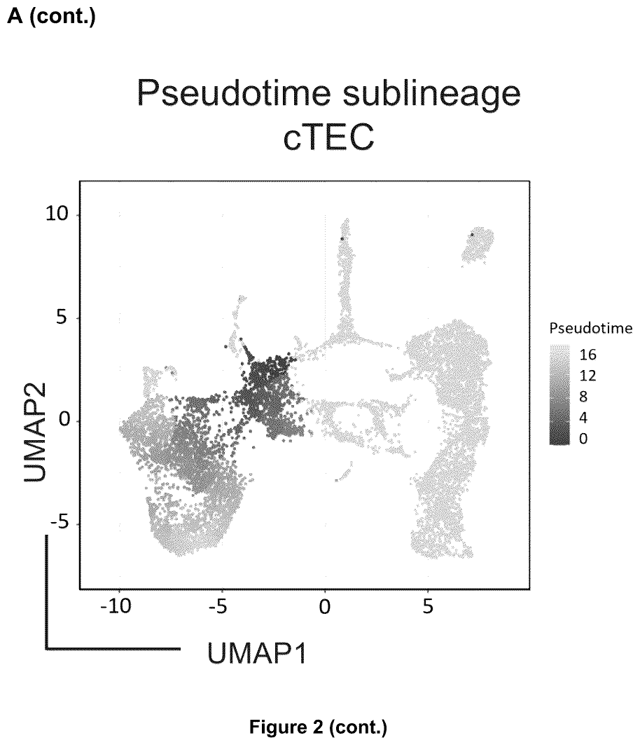

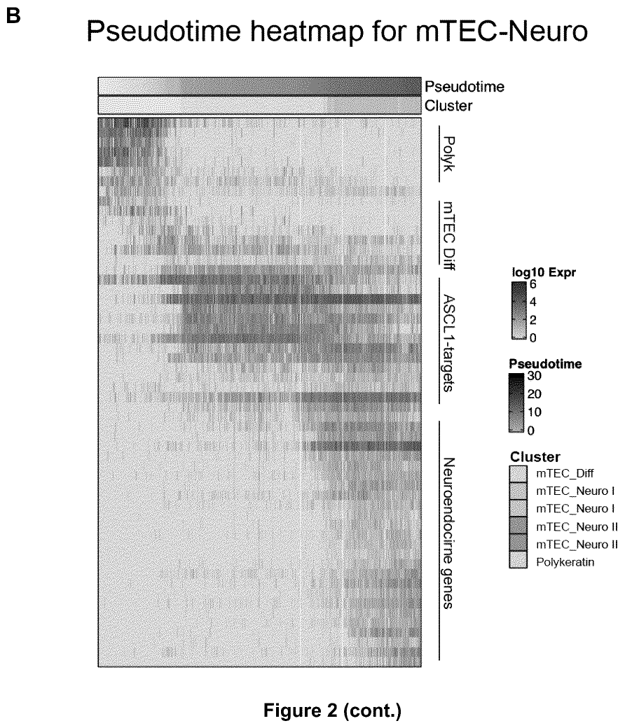

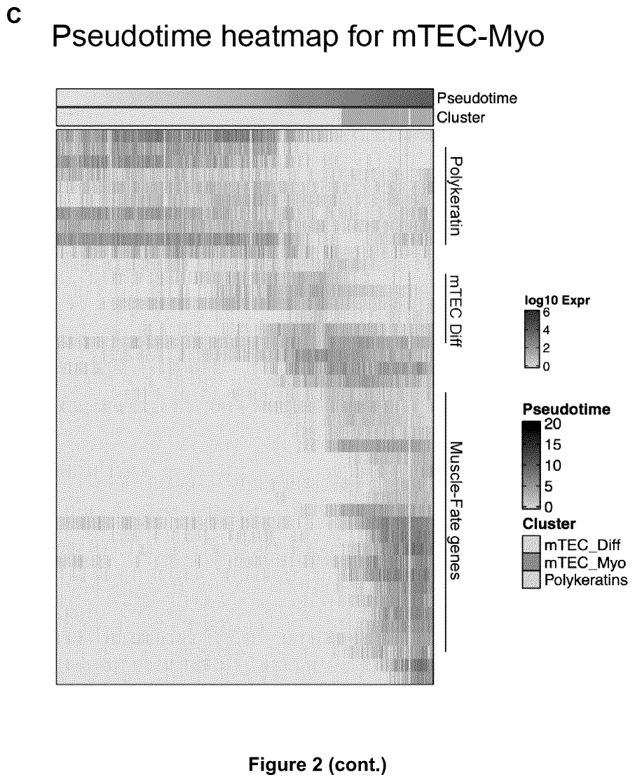

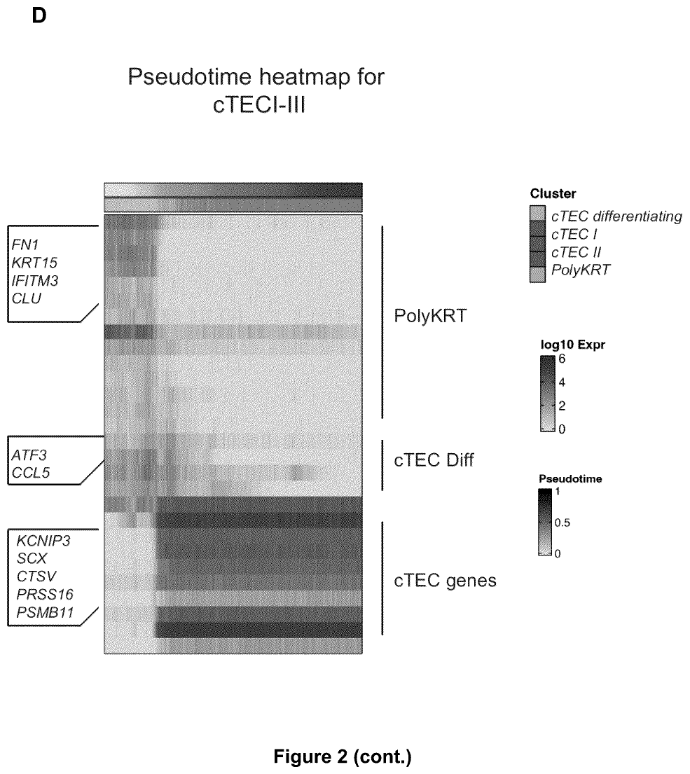

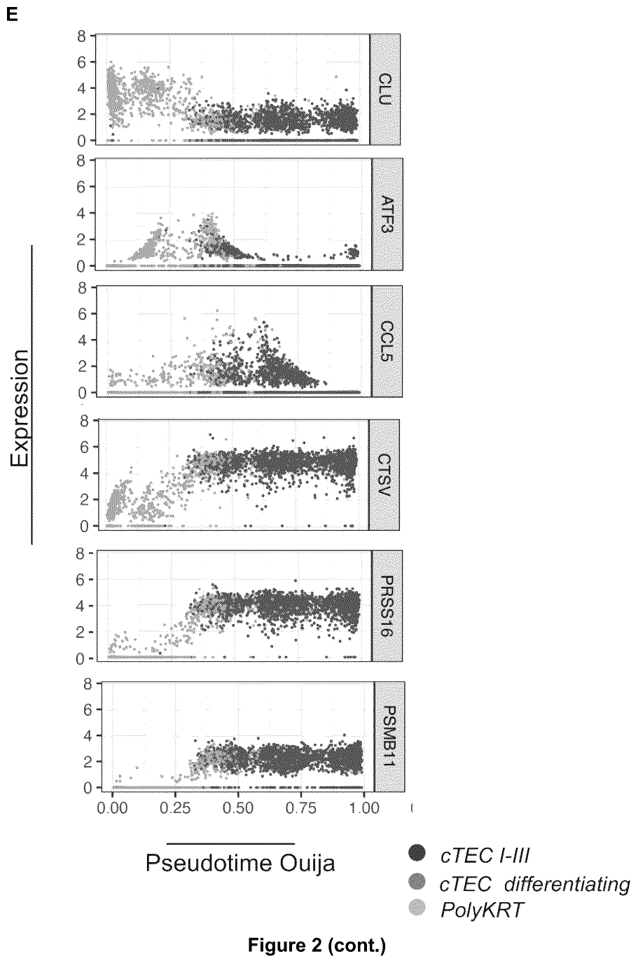

We performed Pseudotime analysis with two independent methods. The Pseudotime UMAP plots obtained with Monocle algorithm highlighted different trajectories from the Polykeratin cluster toward either cTEC-I/III, or mTEC-Myo and mTEC-Neuro differentiated cells passing through the two transition clusters (FIG. 2A). Therefore, we studied which genes were progressively upregulated in these clusters. The feature view UMAP plots for selected genes including KCNIP3, SCX1, and IFIT3, showed they were upregulated in cTEC transition cluster and were driving cTEC fate; similarly, myoid cells (mTEC-Myo) and neuroendocrine cells (mTEC-Neuro-I/II, mTEC-Neuro-III/IV) derived from the transitioning mTEC cluster, characterised by ASCL1 and CLDN3, CLDN4 upregulation. The upregulation of transcription factor ASCL1 that plays a key role for activating neuronal pathways [38,39] was an unexpected finding in a differentiating epithelial population.

The Pseudotime heatmaps represented in more detail the genes involved in the main differences over the whole trajectories towards specialized clusters. mTEC-Neuro (FIG. 2B) and mTEC-Myo (FIG. 2C) heatmaps displayed several sequential steps of differentiation with progressive gene down and upregulation through the transition clusters. Interestingly, cTEC fate was determined by coordinated upregulation of functional cortical genes that remained stably expressed in mature cortex.

We noticed that CD24 was expressed in all mTEC differentiated clusters (HB region, Ionocytes, mTEC-transition, mTEC-Neuro and mTEC-Myo); its expression specific to the medulla was confirmed by immunohistochemistry.

In addition, when we interrogated the main sources of biological variation by Principal Component Analysis (PCA), we observed that Polykeratin cells separated from differentiated clusters in a multidirectional manner toward all specialized clusters including HB region, cTEC I-IV and mTEC-Myo and mTEC-Neuro. This was also supported by category cluster markers displayed in PCA plots.

Thus, these data, obtained with complementary bioinformatic analyses, lend support to the fact that the Polykeratin epithelial population, common to cortex and medulla represents the multipotent, multilineage stem cells of the postnatal thymus.

The Ouija algorithm [40] allowed us to retrospectively confirm the accuracy of the unsupervised Pseudotime and to infer from a set of genes how cTEC fate was acquired. Interestingly, cTEC fate was determined by coordinated upregulation of functional cortical genes that remained stably expressed in the mature cortex as shown by the Pseudotime heatmap (FIG. 2D), which was further supported by selected single gene plots e.g., PSMB11, PRSS16 and CTSV (FIG. 2E). Concomitantly, Polykeratin genes such as CLU, were progressively downregulated and others, e.g., ATF3, CCL5 were transiently expressed in the transition cluster and then downregulated when the cells acquired cTEC identity (FIG. 2E). Thus, acquisition of cTEC fate appeared to be determined by synchronous activation of cortical transcription factors and marker genes (e.g., KCNIP3, SCX, PSMB11, CTSV, PRSS16) that established both differentiation commitment and traits associated with cortical function. Viewed collectively, our data strengthen a conclusion that the Polykeratin epithelial population is common to cortex and medulla and represents stem cells of the postnatal thymus with strikingly high pleiotropic multilineage potency.

### Example 4—Polykeratin Cells can be Prospectively Isolated and are the Clonogenic TEC

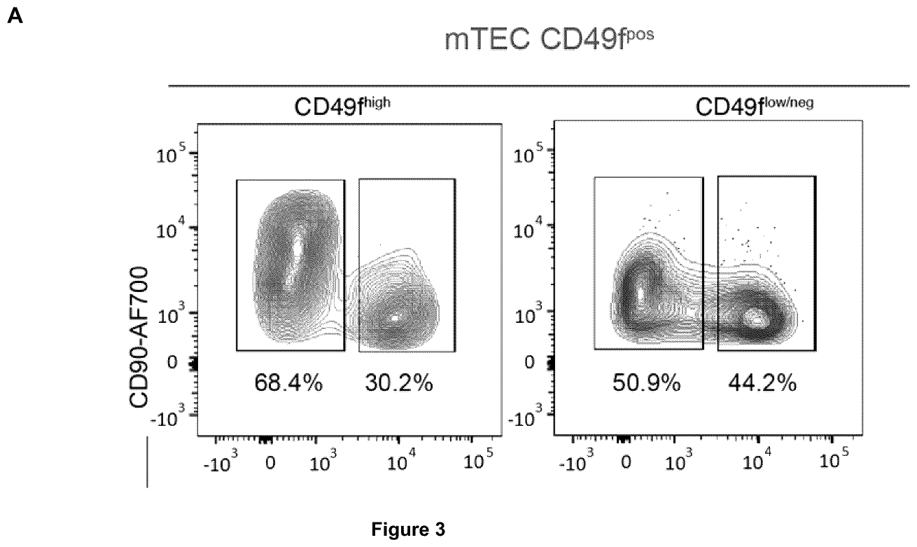

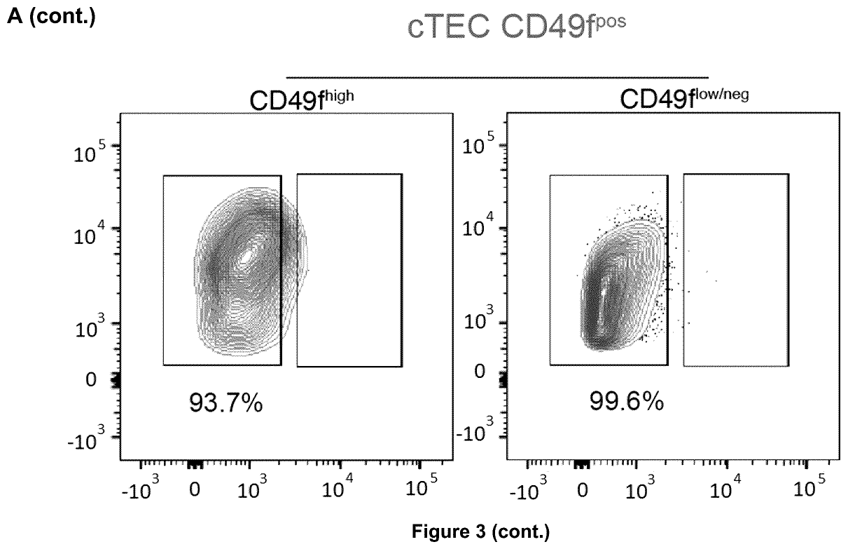

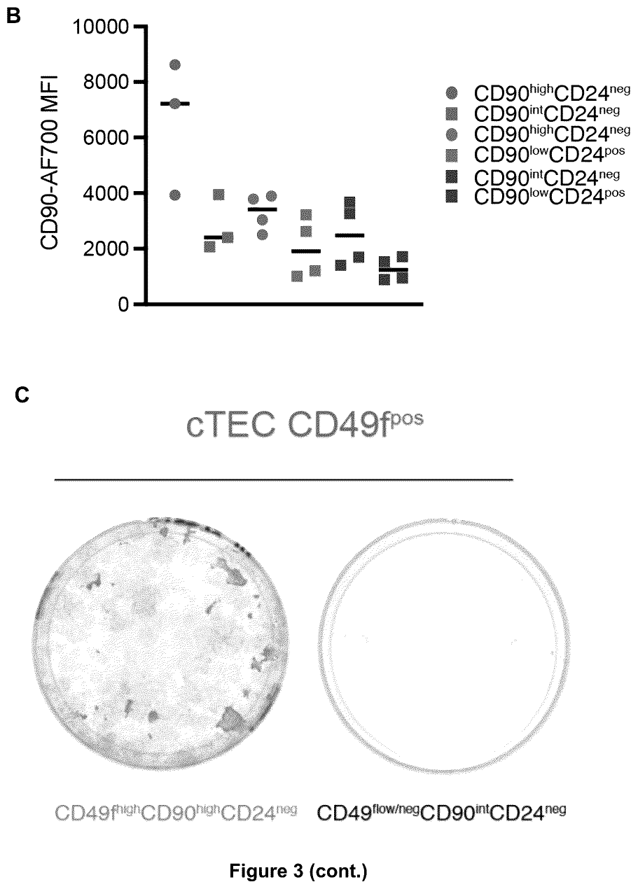

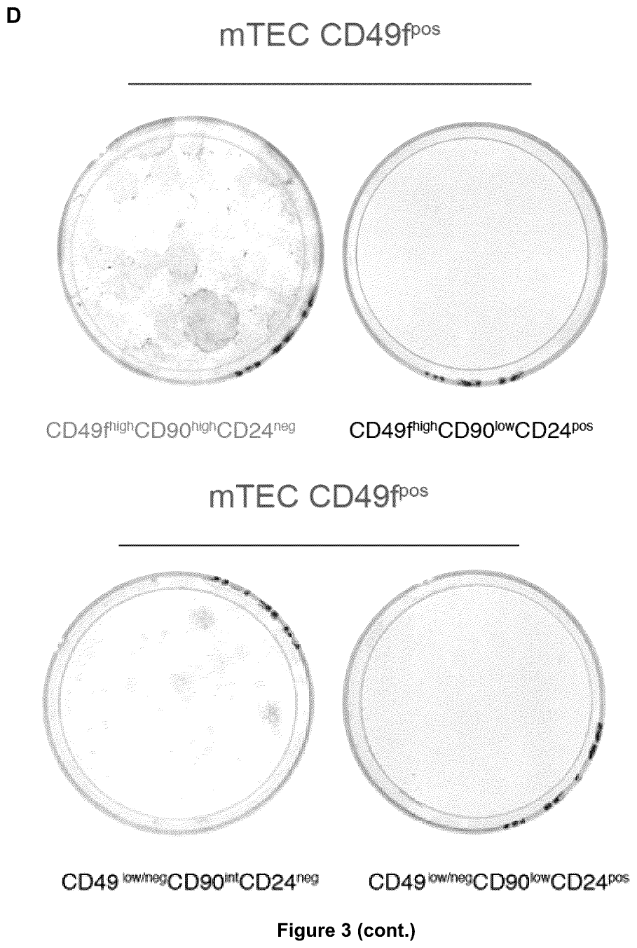

To corroborate that Polykeratin cells are the thymic stem cells, we set out to achieve a high resolution scRNA-seq dataset for each cTEC and mTEC sub-population positive for the surface marker CD49F (ITGA6), reported to enrich for epithelial progenitors in various tissues [41,42]. In the UMAP dotplot, red dots displayed single cells sorted as mTEC-CD49F^{pos} and green dots cells sorted as cTEC-CD49F^{pos}. Clustering analysis of these two populations demonstrated that the Polykeratin and Polykeratin-proliferating signatures were the two main clusters in both cTEC-CD49F^{pos} and mTEC-CD49F^{pos} although they were more abundant in mTEC-CD49F^{pos}. In addition to Polykeratin, cTEC-CD49F^{pos} also contained differentiated cells of cTEC-IV cluster, while mTEC-CD49F^{pos} cells also contained cells of mTEC-Myo and mTEC-Ionocyte clusters. With the aim of defining a panel of surface molecules to purify the Polykeratin subclusters for prospective isolation, we considered that differentiated cells expressed CD24 whereas Polykeratin cells did not (FIG. 3B). We proceeded with multiple enrichment steps of stromal cells and analysed the cells by FACS for CD24 and CD90 (THY1); the latter is a surface molecule that we previously reported to be expressed by TEC with an epithelial-mesenchymal hybrid phenotype [44]. Thus, cortical (CD205^{pos}EpCAM^{neg}) and medullary (CD205^{neg}EpCAM^{pos}) cells were further subdivided based on level of expression for CD49F, CD24 and also CD90 (FIG. 3A) as shown by mean fluorescence intensity (MFI) quantification (FIG. 3B).

These findings led us to adopt the following sorting strategy: two cortical populations were isolated as CD49F^{pos}CD90^{pos}CD24^{neg} and CD49F^{meg} CD90^{int}CD24^{neg}; and four medullary populations as CD49F^{pos}CD90^{pos}CD24^{neg}; CD49F^{pos}CD90^{neg}CD24^{pos}; CD49F^{neg}CD90^{int}CD24^{neg}; CD49F^{neg}CD90^{neg}CD24^{pos}. When all these fractions were assessed independently for clonogenicity in culture, only CD49F^{pos}CD90^{pos}CD24^{neg} cells gave rise to expanding colonies irrespective of their medullary or cortical origin (FIGS. 3C, 3D). The CD49F^{neg}CD90^{int}CD24^{neg} mTEC fraction gave rise to few colonies that could not be subcultured (FIG. 3D). In summary, CD49F^{pos}CD90^{pos}CD24^{neg} defined thymic epithelial clonogenic cells with long-term expanding potential for both the cortex and medulla. Given that their surface molecules excluded cells of differentiated clusters, we now refer to them as cTEC and mTEC clonogenic cells.

### Example 5—scRNA-Sequencing of TEC in Culture Defines a Thymus-Specific Signature

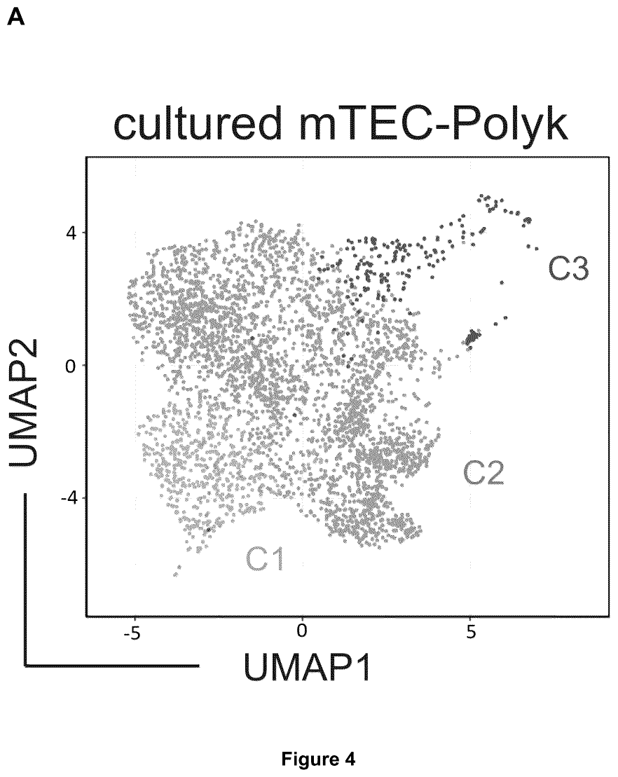

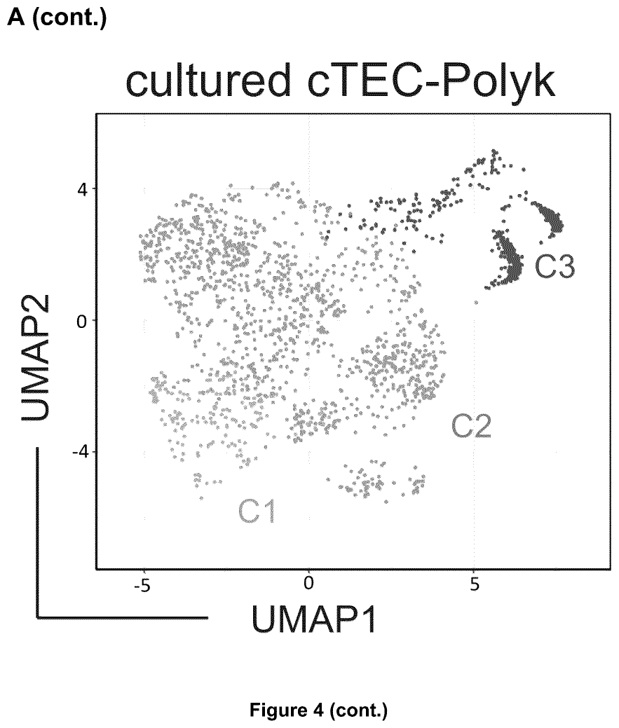

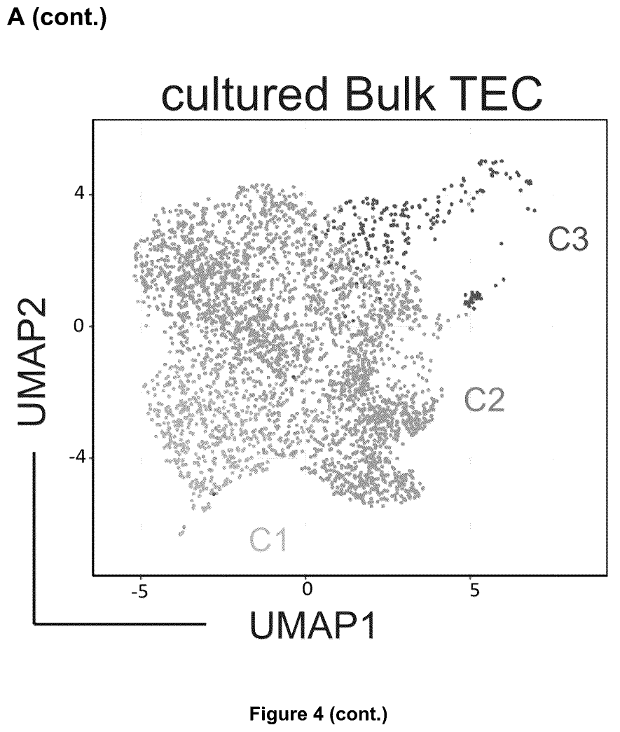

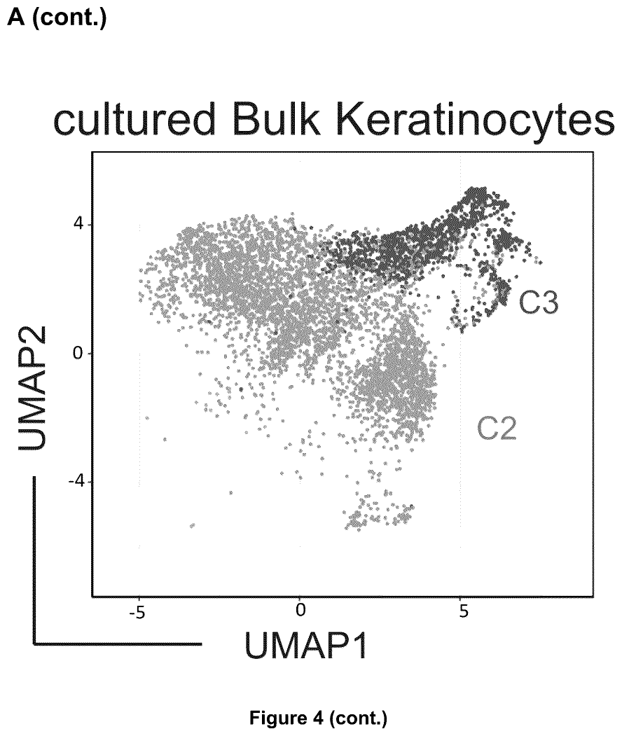

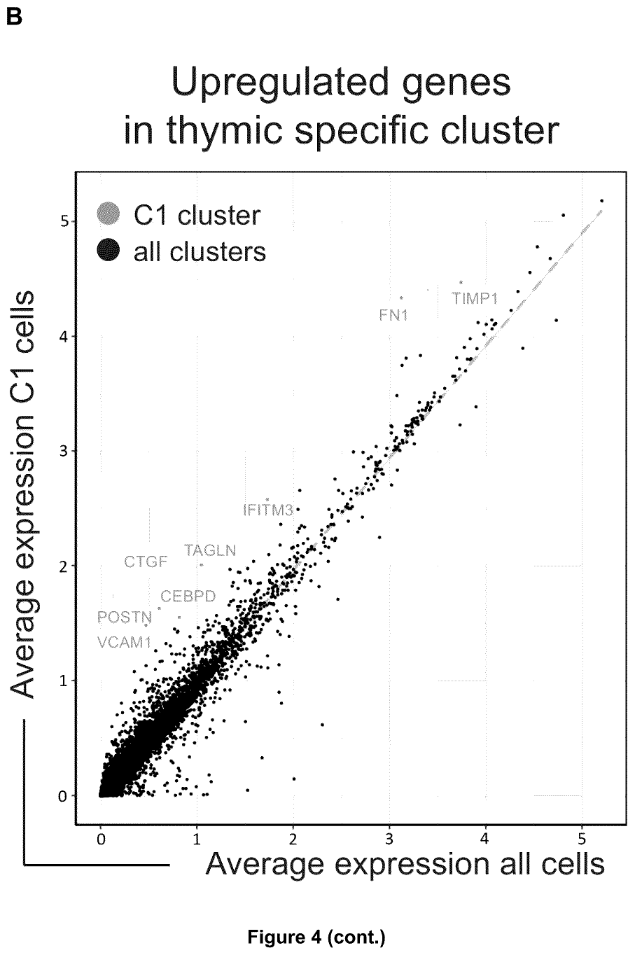

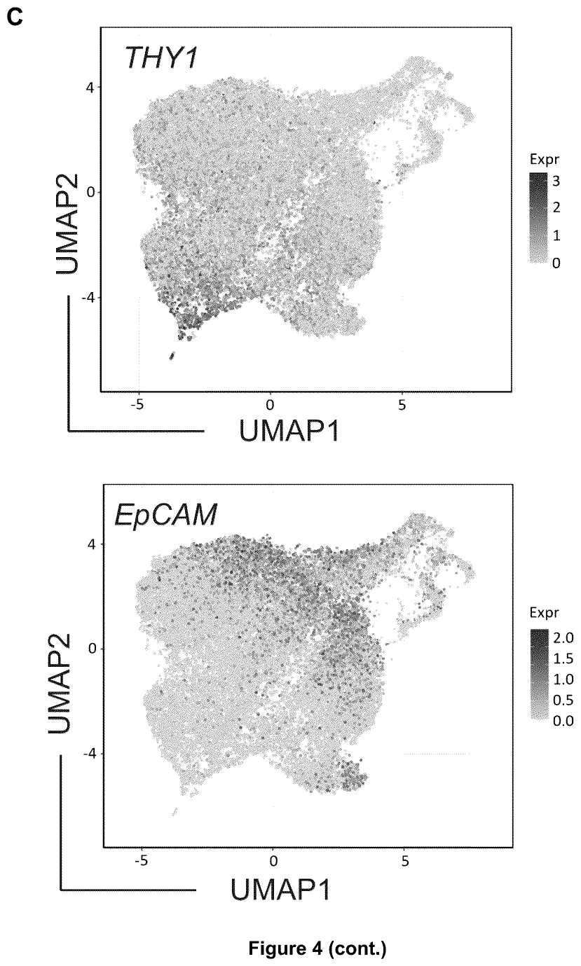

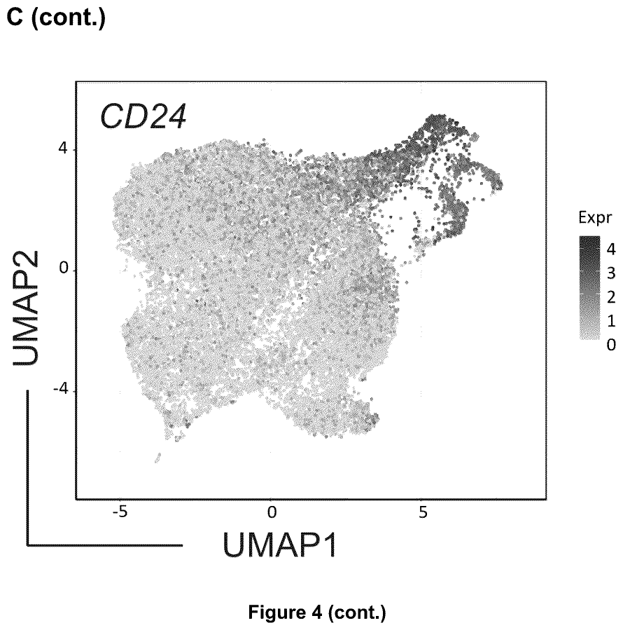

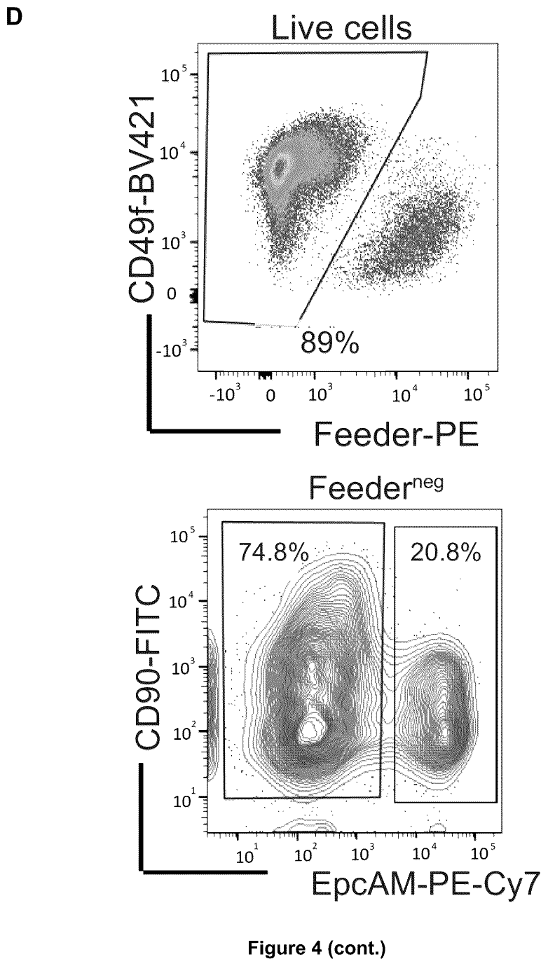

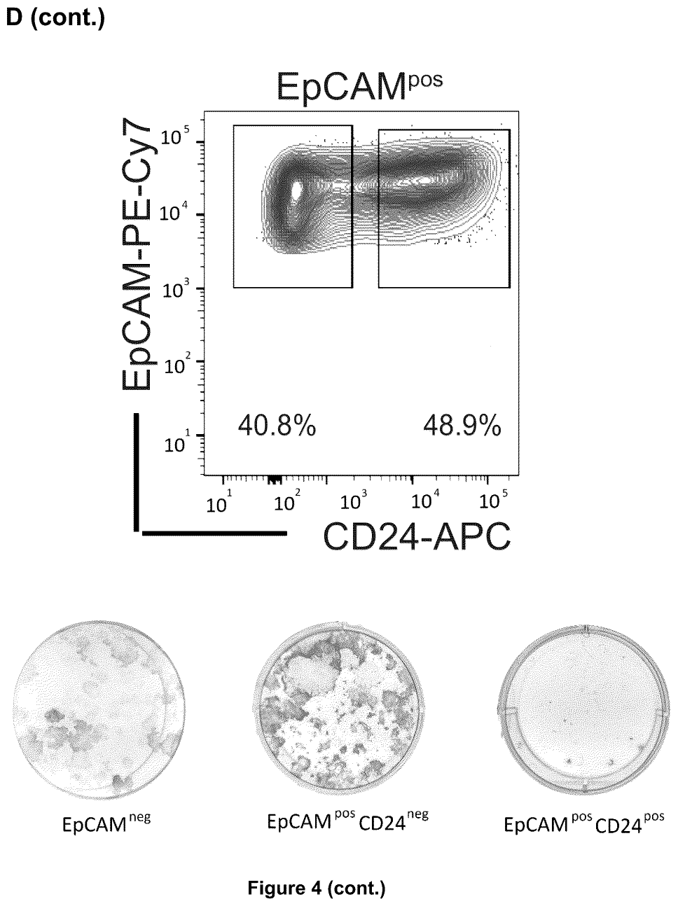

To investigate whether clonogenic epithelial cells retain Polykeratin traits in culture we performed scRNA-sequencing of in vitro expanded cells. We performed scRNA-seq on two biological replicates for each culture type: clonogenic cTEC, mTEC and TEC isolated without prospective isolation a.k.a. bulk cultures (FIG. 4A, B). Such analysis allowed us to address if clonogenic cells would differ depending on their compartment of origin (cortex or medulla) and/or by isolation method. We also included one sample of epidermis-derived clonogenic stem cells with long-term regeneration capacity [15]. Epidermal bulk culture was used as a comparison with thymic cultures. Cells were expanded and sub-confluent cultures at day 5 after plating were harvested and processed for 10× Genomic single cell sequencing. We profiled 2796 and 4297 cells for sorted cTEC-Polykeratin; 2455 and 4297 cells for sorted mTEC-Polykeratin; 5155 and 4787 cells for bulk TEC cultures; and 9478 cells for epidermal bulk cultures. All samples were further processed for sub-clustering to eliminate mouse feeder cells.

All thymic cultures, independently of their derivation, showed comparable profiles in the UMAP plots with identification of three main cell groups (FIG. 4A). Clustering analysis showed that thymic and epidermal cells shared most of the clusters, although a thymic-specific (C1) cluster emerged (FIG. 4A, 4B). The thymic-specific C1 cluster highlighted the atypical epithelial signature expressed by Polykeratin cells in vivo (i.e. FN1, TIMP1, IFITM3, VCAM1) in addition to CD90 (THY1) (FIG. 4C); this is consistent with the above mentioned epithelial-mesenchymal hybrid phenotype [44]. Clusters C2 and C3 were common to TEC and epidermal keratinocyte cultures and expressed markers of stratified and cornified epithelia. Interestingly, cluster C1 (CD90^{pos}, purple dots in feature view plot) did not express markers which were progressively expressed by stratified cluster C2 (EpCAM^{pos}) and then cornified cell cluster C3 (CD24^{pos}) (FIG. 4D).

Therefore, we set out to define the surface molecule profile of TEC clusters in vitro. We analysed by FACS thymic cultures stained with surface markers used above for prospective cell subset isolations: EpCAM, CD49F, CD90 (THY1), and CD24. Cortical surface protein CD205 (LY75) was not expressed by cultured expanding cells either as transcripts or as protein, and consequently was not included in the panel. We excluded mouse 3T3-J2 feeder cells by staining them with a Feeder-PE antibody. Cultured TEC were identified as CD49F^{pos}Feeder-PE^{neg} (FIG. 4). Surprisingly, most of thymic epithelial cells in culture downregulated EpCAM including those coming from EpCAM^{pos} mTEC sorted cells. A large proportion was positive for CD90, consistent with the hybrid epithelial-mesenchymal phenotype. In contrast, CD24 was highly expressed by a subpopulation of EpCAM^{pos} cells (FIG. 4D). Conversely, skin keratinocytes were all CD90^{neg}EpCAM^{pos} and expressed CD24.

We concluded that clonogenic TEC display a thymic-specific EpCAM^{neg} subpopulation characterized by the atypical Polykeratin signature.

### Example 6—Polykeratin Stem Cells in Culture Display a ‘Refractive-Edges’ Morphology

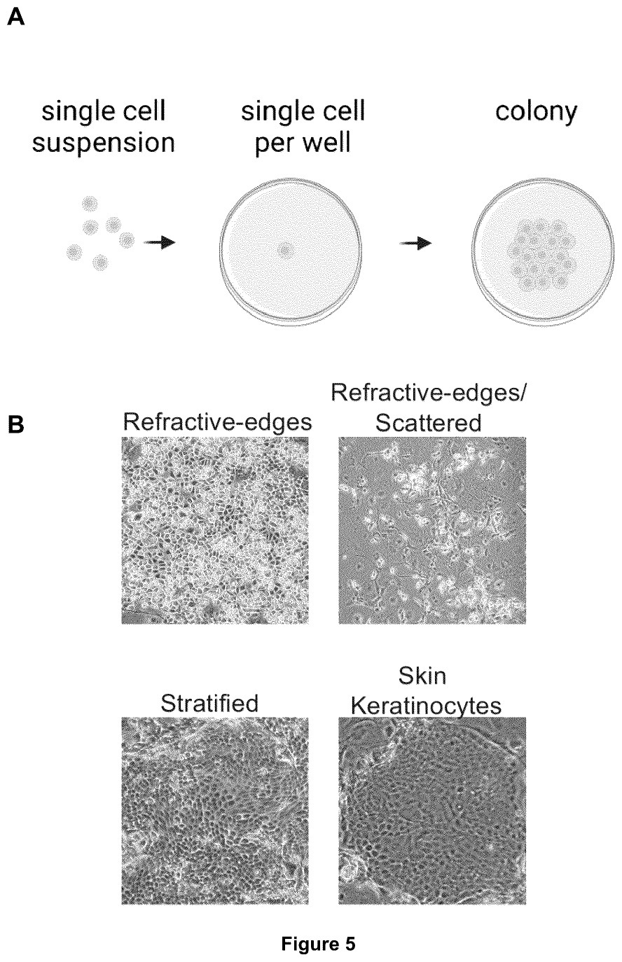

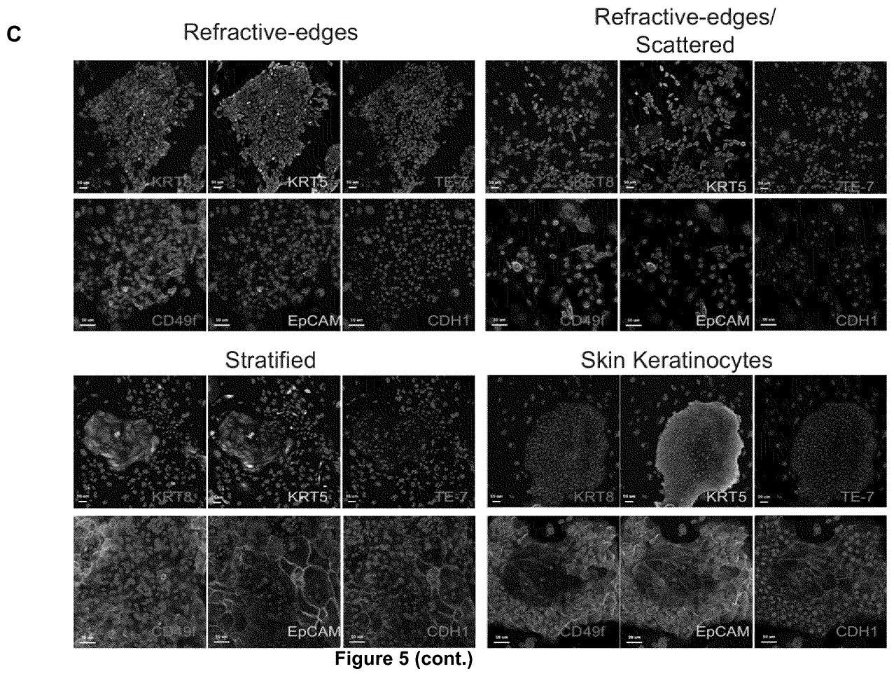

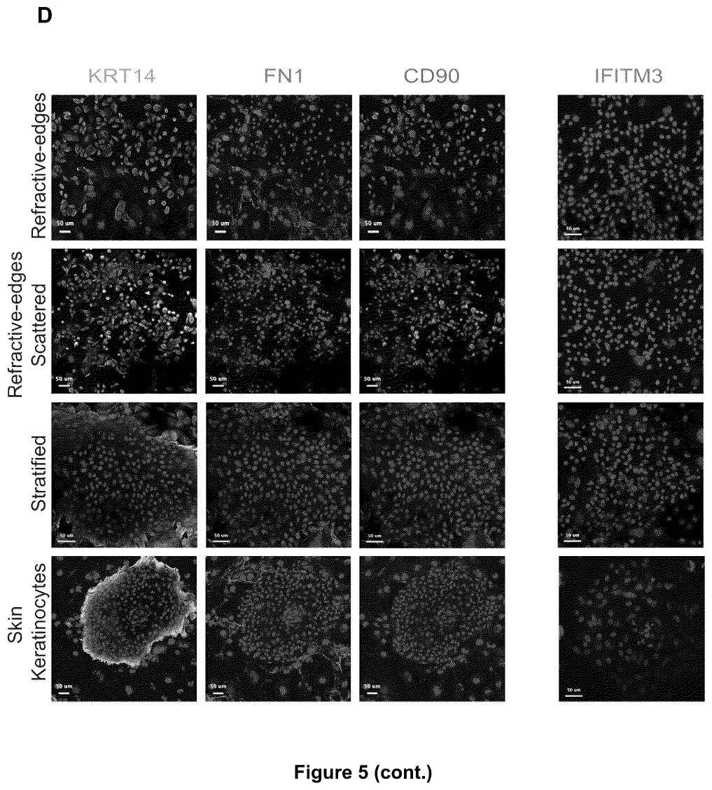

To further investigate the nature of the thymic-specific clonogenic cells, we performed single cell clonal analysis of TEC at limiting dilution (FIG. 5A). This enabled us to classify TEC based on different colony morphologies and Rhodamine-B staining. Rhodamine-B is a dye that is used to evaluate epidermal cell keratinization in cultured cells that correlates with its intensity [43]. Phase contrast images of individual colonies highlighted a morphological heterogeneity: we defined ‘refractive-edges’ colonies that were composed of cells with refringent borders; we named ‘refractive-edges/scattered’ colonies as those containing cells that were highly motile instead of adhering to each other; and another morphology was termed ‘stratified’ for its similarity to the colony morphology of cultured stratified epithelia, e.g. epidermal keratinocytes (FIG. 5B). Colonies that grew and displayed a pile-up differentiation were named ‘aborted’ as keratinocyte aborted colonies. When we analysed them by immunocytochemistry, the expression of EpCAM with additional epithelial and mesenchymal markers such as KRT5, KRT8, E-Cadherin (CDH1) and TE-7, we noticed that all TEC morphologies corresponded to epithelial cells as they co-expressed KRT5/KRT8 (FIG. 5C). However, CDH1 and EpCAM stained only colonies with ‘stratified’ morphology, while TE-7 stained only ‘refractive-edges’ and ‘refractive-edges/scattered’ colonies (FIG. 5C). Based on the co-expression of the TE-7 mesenchymal protein with KRT, we concluded that our previously described motile, hybrid epithelial-mesenchymal phenotype [44] corresponded to ‘refractive-edges’ colonies.

We therefore studied expression of marker genes that identified the Polykeratin cluster in vivo as well as thymic-specific C1 cluster in vitro. We stained these colonies for the newly identified Polykeratin-specific proteins such as FN1, IFITM3 and TIMP1. The results demonstrated expression of these proteins only in ‘refractive-edges’ morphologies, but not in ‘stratified’ colonies of either thymus or epidermis (FIG. 5D). In addition, FACS analysis of cultivated TEC showed expression of another Polykeratin-specific marker VCAM1 only in EpCAM^{neg}CD90^{pos} fraction.

Next, we expanded single clones to study the hierarchical relationship of each cell type. The dimmed Rhodamine B-stained colonies corresponded to refractive-edges cell morphology, while stratified clones were strongly stained with Rhodamine B. These results confirmed that single cells with ‘refractive-edges’ colony-forming capacity were able to generate all morphology types upon subculture while ‘stratified’ produced only ‘stratified’ and terminally differentiated colonies. Thus, the properties of EPCAM^{neg} ‘refractive-edges’ cells were consistent with those expected of a multipotent clonal thymic population in culture and corresponded to the cell type identified as thymic-specific C1 cluster in our scRNA-seq dataset.

### Example 7—Polykeratin Stem Cells Retain Multi-Lineage Differentiation Potency In Vitro

The above Examples describe complementary bioinformatic analyses of the scRNA-seq dataset indicating that Polykeratin cells were capable of multilineage differentiation in vivo. To determine if in vitro expanded Polykeratin SCs retained multilineage differentiation potency, we developed an assay that favoured TEC differentiation, which we named ‘tissoid’, obtained by seeding only one expanded epithelial cell type, with no support from other stromal or haematopoietic cells. The ‘tissoid’ is equivalent to the ‘organoid’ assay to assess lineage differentiation of expanding stem cells, although expansion occurs in 2D instead of 3D.

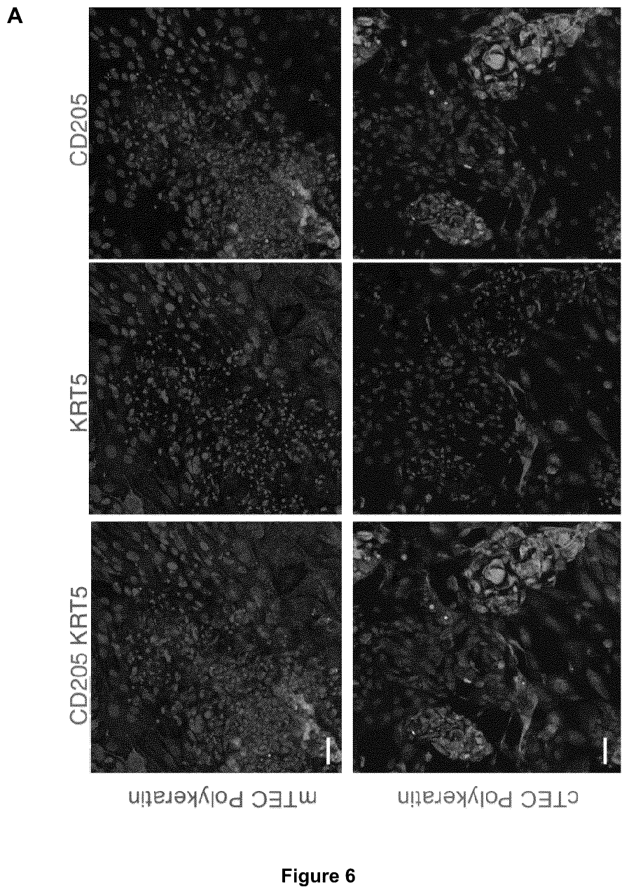

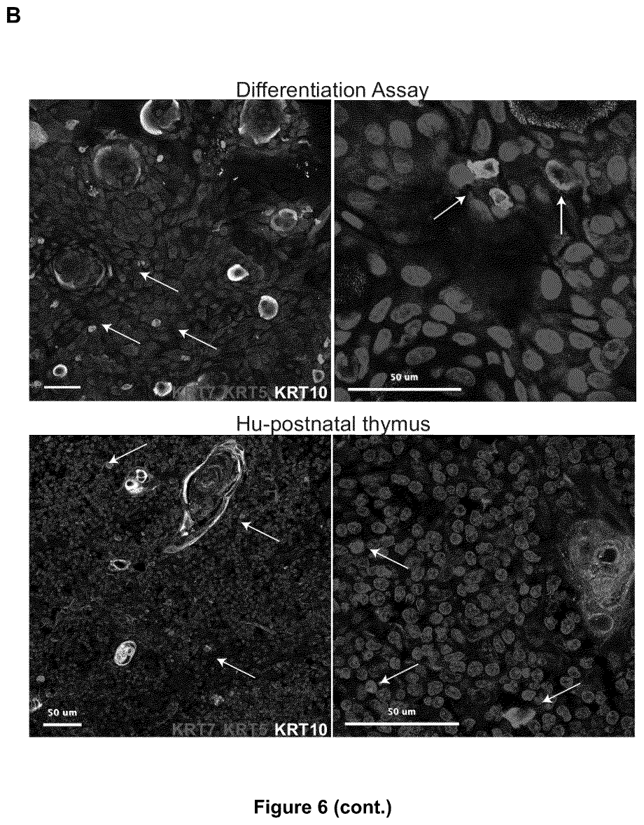

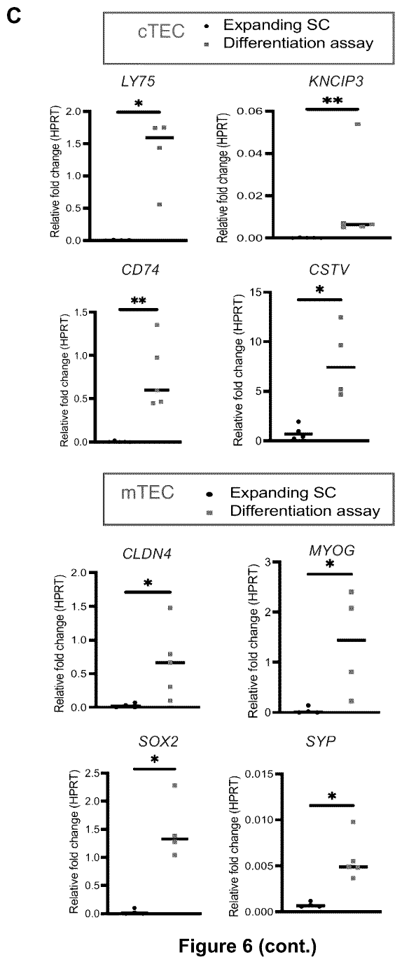

Expanded Polykeratin TEC were seeded on a membrane at high density for two days until confluence. Expansion medium was then substituted by differentiation medium changed every other day for at least 14 days to a maximum of 21 days. Differentiated cells were then either fixed for immunohistochemistry (IHC) or their lysate processed for RT-qPCR analysis. Cortical (CD205^{pos}KRT5^{neg}KRT14^{neg}) and medullary (CD205^{neg}KRT5^{pos}KRT14^{pos}) differentiation was achieved independently from the cell type (cTEC or mTEC Polykeratin) that initiated the culture, medullary and cortical areas were clearly distinguishable and mutually exclusive (FIG. 6A). Of note, cultured cells were able to generate mTEC HB-like regions (KRT10+), sparse ionocytes (KRT7+) similarly to medullary areas of the native thymus (FIG. 6B). Medullary fates were further confirmed by upregulation of mTEC transient cluster (ASCL1, CLDN3, CLDN4), neuroendocrine (SOX2, SOX11, SYP) and myoid (MYOG) cell lineage genes whereas cortical fate by upregulation of CTSV, FOXN1, CD74, CD274 and KCNIP3 genes in differentiated cultures compared to the same stem cells in expansion conditions (FIG. 6C).

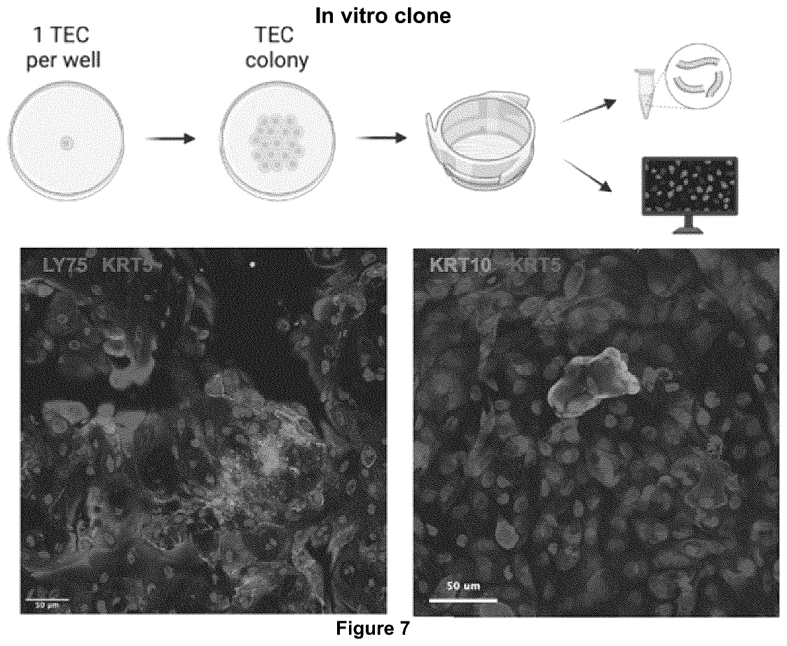

To conclusively demonstrate the intrinsic multipotency of cTEC and mTEC Polykeratin, we expanded single clones and their progenies were assessed by the same assay described above. All clones capable of expansion growth, demonstrated multilineage differentiation potency giving rise to multiple medullary and cortical fates (FIG. 7).

### Example 8—Polykeratin Stem Cells Retain Multi-Lineage Differentiation Potency In Vivo

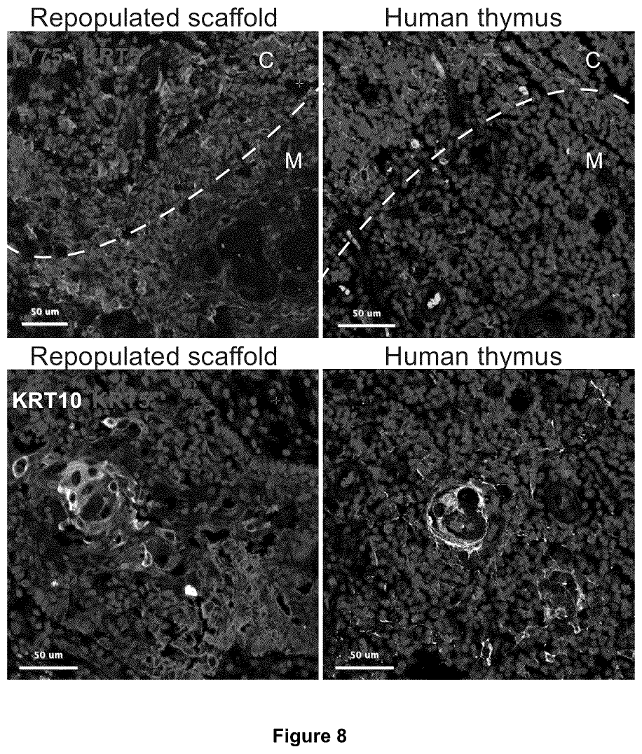

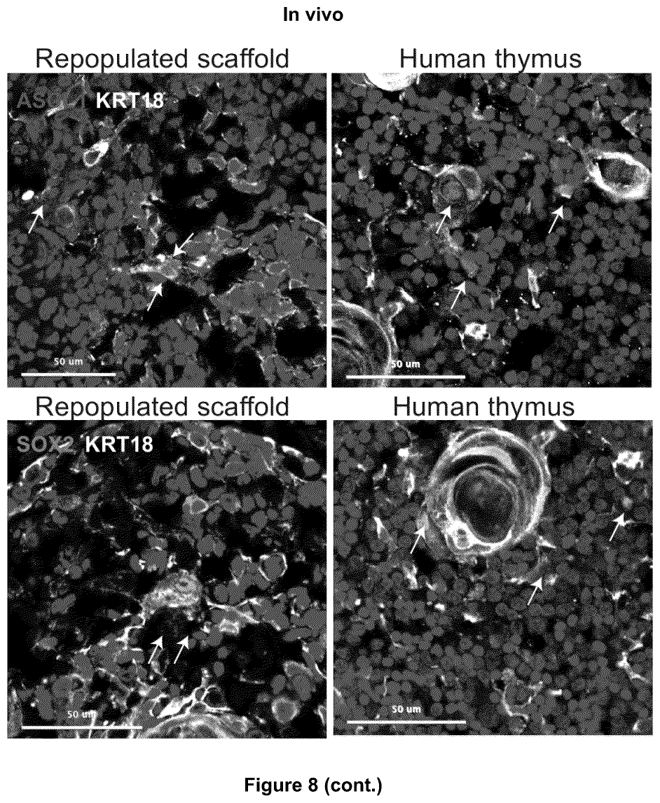

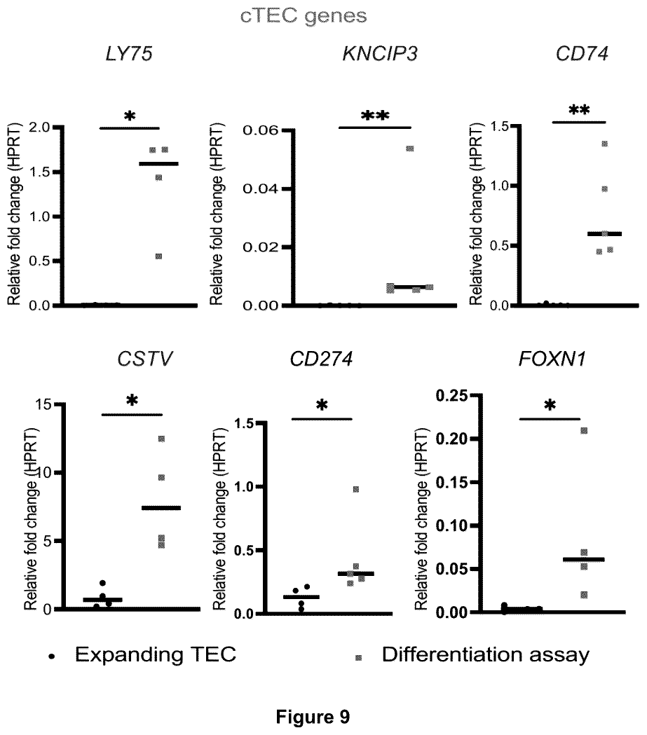

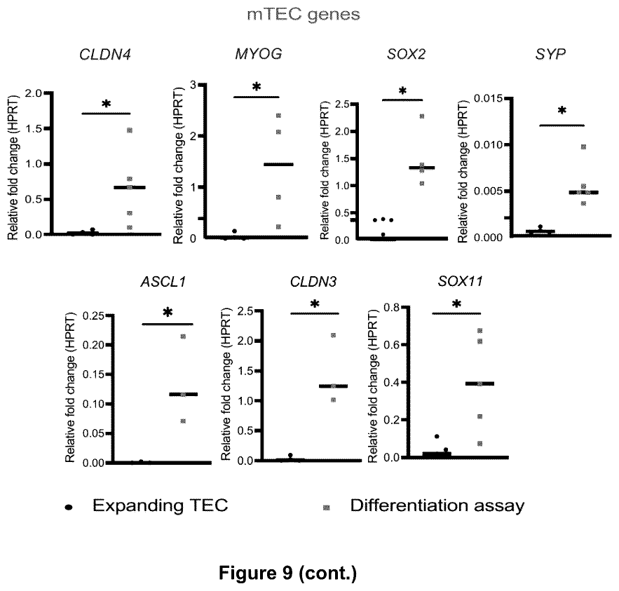

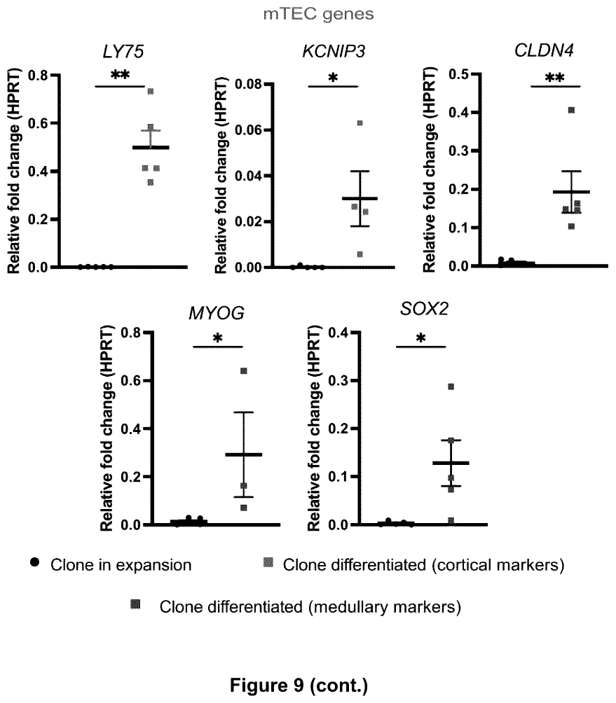

We assessed whether multilineage differentiation of clonogenic SC would also be achieved in vivo using a whole organ thymus reconstitution assay that we previously developed [44]. In this assay, clonogenic TEC (as defined above) were injected together with cultured thymic interstitial cells (TIC) into acellular thymic scaffolds subsequently transplanted into humanized NSG and NSG-Nude (athymic) mice where they become vascularized and, if functional, can attract hematopoietic progenitors from reconstituted bone marrow. Grafts were harvested at 10- and 16-weeks post-transplantation (wpt). Thymus reconstitution was characterized by progressive maturation of the stroma and its compartmentalization into cortical and medullary regions, while seeding of hematopoietic progenitors followed by thymocyte (CD3+ cells) development demonstrated the appropriate functioning of the reconstituted organs. Indeed, thymocyte repopulation increased from 10 to 16 wpt. Thymus reconstitution by expanded polykeratin TEC was characterized by stroma compartmentalized into cortical and medullary regions including HB formation, medullary progenitors (ASCL1⁺) and mature neuroendocrine cells (SOX2⁺) which phenocopied the tissue organization of the native thymus (FIG. 8).

Thus, we could conclude that, similarly to PolyKRT in vivo, clonogenic Polykeratin TEC retained multipotency after isolation and in vitro expansion with the capacity to reconstitute multiple thymic compartments even from a single clone. These are akin to the defining criteria for human multipotent SC proven capable of self-organization and organ reconstitution. We concluded that clonogenic Polykeratin stem cells retained multipotency upon isolation and in vitro expansion.

### Example 9—Subcapsular and Perivascular Spaces Represent the In Vivo Niche of Polykeratin Cells

Noting the expression of ECM-related genes by Polykeratin cells, we investigated their protein expression within the human postnatal thymus with the intention of localising them in vivo. We performed immunohistochemistry on thymus sections for EpCAM, CD49F (ITGA6), KRT13, KRT14, KRT15, KRT17, KRT18, as well as the above mentioned Polykeratin-specific proteins FN1, IFITM3 and TIMP1.

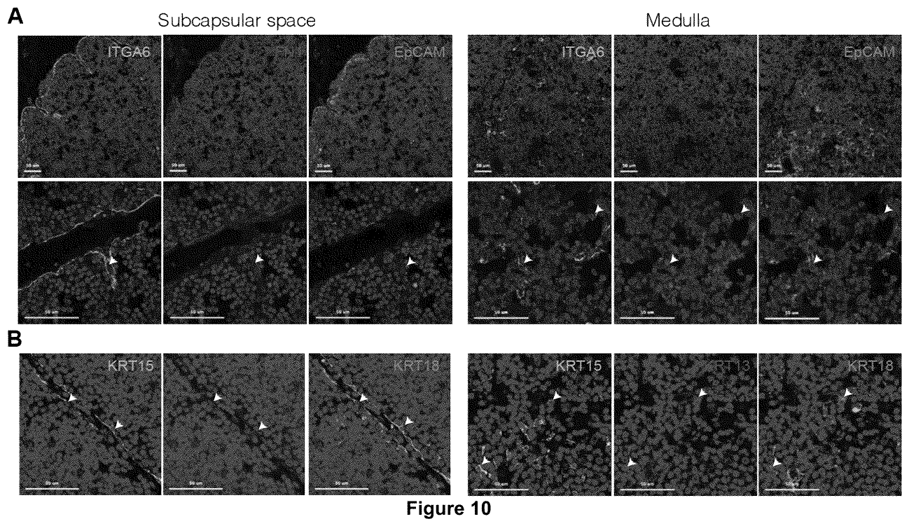

Both medullary and cortical areas were screened and imaged by confocal microscopy. Triple co-localization of EpCAM, CD49F, FN1 or IFITM3 and CD49F, FN1 and TIMP1 were detected in rare cells in subcapsular areas and in scattered cells within the medulla (FIG. 10A). Polykeratin cells displayed a strong signal for CD49F and we further confirmed their epithelial nature with EpCAM immunostaining that was bright in the medulla and dimmer in the cortex in line with their FACS profile. Importantly, they co-expressed the novel markers IFITM3 and FN1 (FIG. 10A). In addition, triple co-localization of cytokeratins (KRT13, KRT18 and KRT15 or KRT17) displayed a similar pattern of expression in both subcapsular and medulla regions (FIG. 10B).

We next investigated the pattern of expression of TP63 transcription factor (TF) and of its isoform ΔNTP63α expressed in the basal layer of the epidermis and previously associated with stemness in various epithelia including that of the thymus [12,14]. Our data showed that TP63 antibody (recognising all TP63 isoforms) had a broader expression pattern in the thymus with some brightly stained cells in both the cortex and medulla that co-localized with KRT17 and KRT18. ΔNTP63α isoform had a more restricted pattern of expression with a distribution reflecting that of the Polykeratin cells.

The ECM and in particular the basal laminae represent important components of the epithelial stem cell niches [45]. We performed 3D reconstruction of confocal images of 300 μm thick sections for FN1 which is one of the main ECM components (also produced by the Polykeratin cells) in order to define the spatial distribution of the basal laminae of the postnatal human thymus. FN1 immunostaining clearly defined the subcapsular and the perivascular spaces within the cortex and medulla, thus suggesting that the basal laminae may represent the in vivo niches of the thymic SC.

We interrogated our scRNA-seq dataset and found that the Basal Cell Adhesion Molecule (BCAM) was one of the genes defining the Polykeratin signature. BCAM is the receptor of Laminin-A5 (LAMA5), an ECM glyco-protein of the basal laminae [46,47] and we confirmed its expression in the same areas of Polykeratin cells. Therefore, we set out to isolate BCAM-expressing cells using our serial enrichment and sorting strategy. Sorted BCAM^{pos}cTEC (EpCAM^{neg}CD205^{pos}) and BCAM^{pos}mTEC (EpCAM^{pos}CD205^{neg}) gave rise to long-term expanding cultures.

We concluded that Polykeratin cells localised in the subcapsular and perivascular niches in vivo are the long-lived thymic stem cells.

### Example 10—BCAM as an Identifying Marker of Clonogenic Thymic Stem Cells

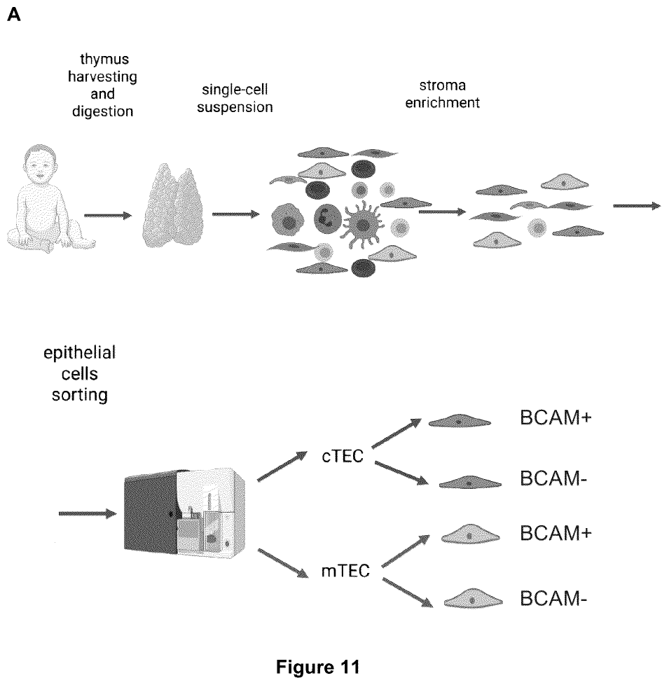

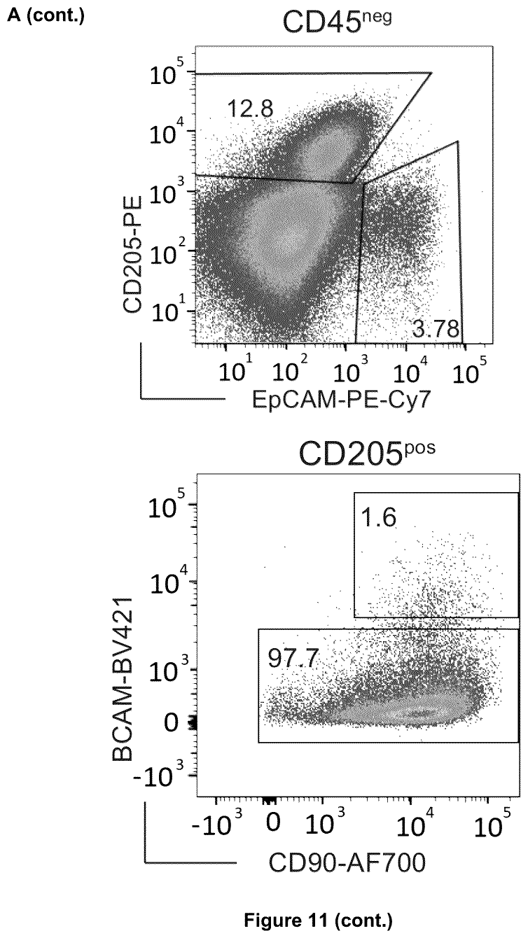

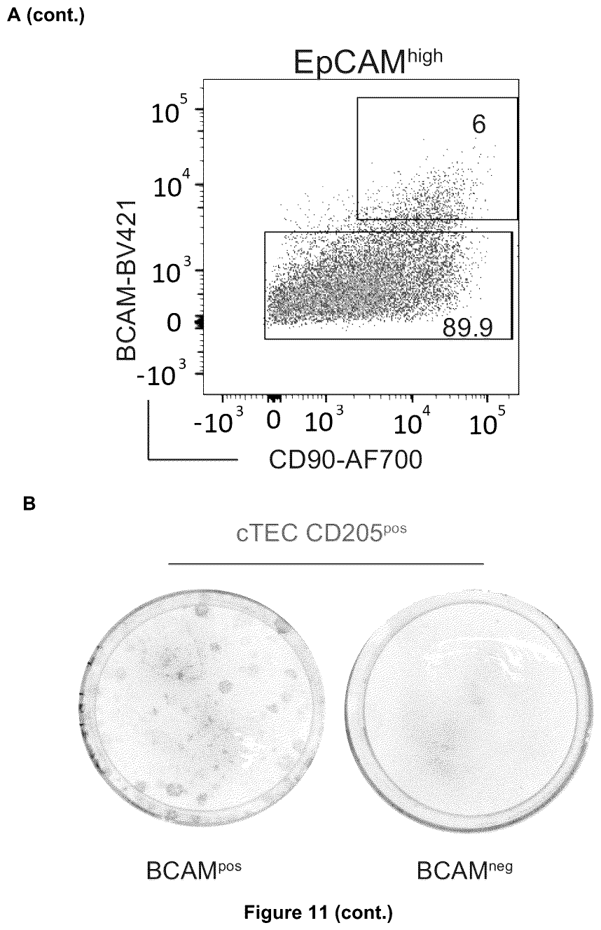

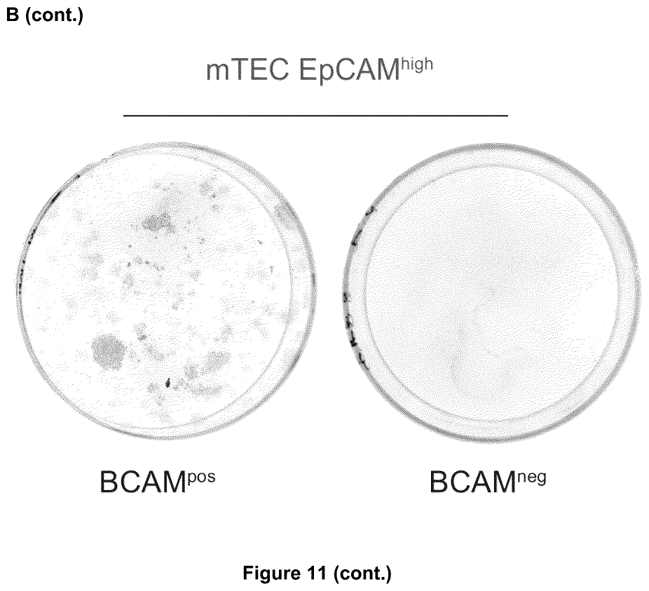

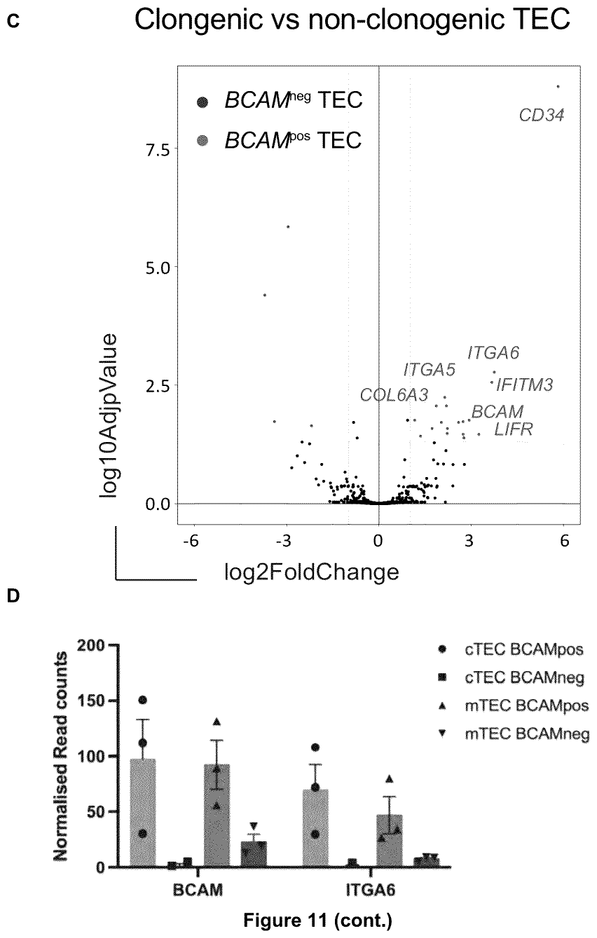

We set out to purify both cortical and medullary Polykeratin cells based on the level of expression of the newly validated basal cell adhesion molecule, BCAM. Whole thymic tissues were dissociated into single cells and stained for cortical (CD205^{pos} EpCAM^{low}) and medullary (CD205^{neg} EpCAM^{high}) surface markers and further subdivided into BCAM^{pos} and BCAM^{neg} TEC (FIG. 11A).

We endeavoured to isolate BCAM-expressing cells by FACS and found that only BCAM^{pos} cells in both cortex and medulla were clonogenic and gave rise to epithelial colonies able to be sub-cultured and expanded (FIG. 11B). Completely consistent with the spatial phenotyping above, our flow cytometry studies also confirmed that the medulla contains a larger proportion of clonogenic cells (FIG. 11A). Freshly isolated clonogenic (BCAM^{pos}) and specialized, non-clonogenic (BCAM^{neg}) TEC, sorted cells were subjected to nCounter® automated analysis (NanoString) that allows multiplex gene expression profiling.

Of note, clonogenic BCAM^{pos} cells when freshly isolated, retained some of their cortical or medullary identity reflected by differential expression of compartment-specific genes such as cortical CTSV, FOXN1, PSMB11, SCX, LY75 and medullary CCL21, EpCAM, CLDN3 and CLDN4, respectively. Of note, however, the volcano plot displaying the differentially expressed genes (DEG) between clonogenic and non-clonogenic TEC provided an independent confirmation, using an independent method, of Polykeratin gene expression in the clonogenic fractions, together with BCAM and ITGA6 adhesion molecules (FIG. 11 A, B).

### CONCLUSIONS

Our data reveal that the postnatal thymus harbors bona fide epithelial stem cells (SC) with multilineage differentiation potency. These cells are characterized by a hierarchical differentiation where multipotent stem cells give rise to different progenitors that represent intermediate stages of differentiation, including cortical differentiating cells that upregulate CD205, CTSV, FOXN1 and KCNIP3. Regarding medullary fate, we observed a ‘transition’ cluster of cells that upregulate CLDN3 and CLDN4, previously reported to be expressed in mTEC restricted progenitors [48]. These cells also upregulate ASCL1, a basic helix-loop-helix (BHLH) transcription factor that is involved in the regulation of neuroendocrine cell development in several organs (e.g., lung and gut).

In fact, our thymic epithelial SC are multipotent and do not express most of the genes previously associated with the progenitor phenotype (e.g. FOXN1 or CLDN3/4). Instead, they lie upstream and are defined by gene expression signatures that reflect their capacity to sustain multiple functions, resist stress and insults, and reside in “strategic” areas where they might sense tissue tension/modifications (subcapsular) and metabolite supply (perivascular). Our data support a new model whereby postnatal SC can be isolated from both cortex and medulla compartments, based on common features and surface molecules. In vivo they give rise to both cortical and medullary specialized cell types, including those that are not obviously epithelial in nature, including neuroendocrine and myoid cells that were previously considered of uncertain origin [27]. The finding of SC with multilineage differentiation and presence of different types of progenitors may be crucial to understand the high heterogeneity of thymic epithelial tumours (i.e. thymoma and thymic carcinoma), often associated also to autoimmunity [49].

Of note, once isolated, thymic SC could be extensively expanded, akin to stem cells of constantly renewing tissues such as the epidermis. This seems paradoxical given that the epidermis is characterized by high cell turnover and is entirely replaced every three weeks lifelong, whereas the thymic epithelium is not actively proliferating and the thymus itself involutes with progressively decreasing functional outputs throughout postnatal life.

In addition to sharing some key properties such as a profound in vitro proliferative capacity, the thymic epithelial SC displayed some unique traits that provide new important insight into epithelial stem cell biology.

First, they co-express several cytokeratins (KRT) that individually define specific lineages of simple, stratified, or glandular epithelia; thus, thymic SC display a unique Polykeratin signature so far described among epithelia. This reflects a promiscuous but regulated trait for proteins such as KRT that are typically known for their specificity and lineage determination [50,51]. This might underpin the plasticity of epithelial stem cells—the capacity to change or increase their potency during differentiation—that has been reported for thymic epithelial cells in the context of transplantation [52].

Second, thymic epithelial stem cells constitutively express several genes involved in immune and 10 inflammatory responses such as CLU, CEBPD and IFITM3. For example, IFITM3 likely represents an increased resistance of these cells to viral infections, a property that has been observed in other stem cells [53]. Furthermore, IFITM3 may have roles that go beyond its classical antiviral activity such as regulation of epithelial-mesenchymal transition (EMT), a process important in oncogenesis and cancer progression [54]. Indeed, its constitutive expression in unstimulated epithelial stem cells is striking when compared to its strictly regulated expression in infected or inflamed tissues or in non-epithelial stromal cells [54,55,56]. Nonetheless, we note that in vivo the thymus is an unique site of constitutive Type I IFNα expression [57] that may contribute to IFITM3 regulation.

Third, thymic multipotent SC localize in subcapsular and perivascular microenvironments where they contribute to their niche by producing extracellular matrix (ECM); they also express ECM-binding proteins, such as CD49F, important for polarization and adherence to the basal lamina. This may reflect an architectural difference from classic epithelia where mono- or pluri-epithelial layers organize on a basal lamina that separates them from the underlying mesenchyme. The thymic 3D meshwork is in fact characterized by the interconnection of epithelial and interstitial cells, which creates a unique microenvironment for thymocyte migration and development. It is worth noting in this context that TEC also express mesodermal markers such as TE-7, Vimentin and CD90 which makes them an atypical epithelium with high motility capacity [44]. The importance of epithelial adhesion to specific ECM for sternness maintenance is further demonstrated by the capacity of proteins like FN1 to inhibit human keratinocytes terminal differentiation [58]. Therefore, the niche ECM appears to share molecular features among different epithelia (including thymus); however, the unicity of thymic epithelial SC, is represented by the capacity of producing their own niche ECM and secreted proteins (e.g. FN1, TIMP1) which are mainly produced by the supporting stroma in other organs.

Finally, thymic SC can be prospectively isolated from different anatomical compartments based on the expression of surface molecules such as EpCAM, CD49F, CD90 and CD24. Alternatively, BCAM expression can be used to isolate clonogenic thymic stem cells. Once seeded in culture, they are activated and extensively expand in vitro where they retain their Polykeratin feature and the capacity of multilineage differentiation independently of their origin or isolation method. Critically, this supports the view that the capacity for thymic homeostasis and regeneration reside in the same (Polykeratin) population. These multipotent thymic-specific stem cells are easily identifiable by their characteristic refractive-edges morphology in culture, and they share the same Polykeratin features identified in vivo independently of their isolation method. This has important consequences for their future use in cell replacement therapies (i.e. transplantation in athymic patients) as minimal manipulation during isolation would reduce stress-induced senescence [58] and facilitate clinical protocols. Bulk cultures are indeed the current method for isolation of both epidermal and limbal SC in clinical use [59,60].

Exploring how to control thymic epithelial SC activation and differentiation capacity in diseased or aged thymi in vivo may offer the means to regenerate and/or rejuvenate the thymic function in several disease settings, particularly in older persons. By studying how Polykeratin cells and their niche change during thymus progressive atrophy and how they respond to exogenous factors both in vivo and in vitro, we will be able to study the mechanisms of thymic involution and design new strategies for increasing thymic output e.g. to augment vaccination responses in vulnerable subjects or improve the immune response against cancer.

### EQUIVALENTS AND SCOPE

Those skilled in the art will appreciate that the present invention is defined by the appended claims and not by the Examples or other description of certain embodiments included herein.

Similarly, the singular forms “a”, “an”, and “the” include plural referents unless the context clearly dictates otherwise.

Unless defined otherwise above, all technical and scientific terms used herein have the same meaning as commonly understood by one of ordinary skill in the art to which this invention belongs. Any methods and materials similar or equivalent to those described herein can also be used in the practice or testing of the present invention. Generally, nomenclatures used in connection with, and techniques of, cell and tissue culture, molecular biology, immunology, genetics and protein and nucleic acid chemistry described herein are those well-known and commonly used in the art, or according to manufacturer's specifications.

All publications mentioned herein are incorporated herein by reference to disclose and describe the methods and/or materials in connection with which the publications are cited.

The invention is also described in the following numbered clauses.
1. An isolated thymic epithelial stem cell, wherein the isolated thymic epithelial stem cell is BCAM^{pos}, CD49F^{pos}, CD90^{pos} and CD24^{neg}.
2. The isolated thymic epithelial stem cell of clause 1, wherein the isolated thymic epithelial stem cell is BCAM^{pos}, CD49F^{high}, CD90^{high} and CD24^{neg}.
3. The isolated thymic epithelial stem cell of clause 1, wherein the isolated thymic epithelial stem cell expresses at least one cytokeratin gene.
4. The isolated thymic epithelial stem cell of clause 1 or clause 2, wherein the at least one cytokeratin gene is selected from the group consisting of KRT5, KRT8, KRT13, KRT14, KRT15, KRT17, KRT18, and KRT19.
5. The isolated thymic epithelial stem cell of any one of clauses 1 to 3, wherein the isolated thymic epithelial stem cell expresses KRT13, KRT18, and KRT15.
6. The isolated thymic epithelial stem cell of any one of clauses 1 to 3, wherein the isolated thymic epithelial stem cell expresses KRT13, KRT18, and KRT17.
7. The isolated thymic epithelial stem cell of any one of clauses 1 to 3, wherein the isolated thymic epithelial stem cell does not express KRT7, KRT1, KRT10, KRT4, KRT16, KRT23 or KRT6A.
8. The isolated thymic epithelial stem cell of any one of clauses 1 to 6, wherein the isolated thymic epithelial stem cell further expresses at least one selected from the group consisting of: EPCAM, CD49F, FN1, TIMP1, IFITM3, VCAM1, CEPBD, CLU, CCL19, CH25H, COL7A1, CTGF, APOE, FGFR2, BOC, ITGA5, SOX17, LIFR, YAP1, PTGDS, CD34, VWF, SPARC, CAV-1, EPAS-1, TIMP3, COL4A2, COL5A1, COL6A3, TP63 (for example ΔNTP63α) and cMYC.
9. The isolated thymic epithelial stem cell of any one of clauses 1 to 7, wherein the isolated thymic epithelial stem cell further expresses EPCAM, CD49F, and FN1.
10. The isolated thymic epithelial stem cell of any one of clauses 1 to 7, wherein the isolated thymic epithelial stem cell further expresses EPCAM, CD49F, and IFITM3.
11. The isolated thymic epithelial stem cell of any one of clauses 1 to 7, wherein the isolated thymic epithelial stem cell further expresses CD49F, FN1, and TIMP1.
12. The isolated thymic epithelial stem cell of any one of clauses 1 to 11, wherein the isolated thymic epithelial stem cell further expresses COL7A1 and CTGF.
13. The isolated thymic epithelial stem cell of any one of clauses 1 to 12, wherein the isolated thymic epithelial stem cell expresses at least one selected from the group consisting of CCNA2, AURKB, FOXM1, ANLN, LMNB1, HMGB2, or combinations thereof.
14. An isolated thymic epithelial stem cell, wherein the isolated thymic epithelial stem cell is capable of ex vivo self-renewal.
15. An isolated thymic epithelial stem cell, wherein the isolated thymic epithelial stem cell is clonogenic.
16. An isolated thymic epithelial stem cell, wherein the isolated thymic epithelial stem cell exhibits long-term expansion capacity in vitro.
17. The isolated thymic epithelial stem cell of clause 16, wherein the isolated thymic epithelial stem cell is capable of at least 15 population doublings in vitro.
18. An isolated thymic epithelial stem cell, wherein the isolated thymic epithelial stem cell is capable of differentiating into cortical thymic epithelial cells and/or medullary epithelial cells.
19. The isolated thymic epithelial stem cell of and preceding clause, wherein the thymic epithelial stem cell is a cortical thymic epithelial cell and is CD205^{pos}KRT5^{neg}KRT14^{neg}.
20. The isolated thymic epithelial stem cell of any preceding clause, wherein the thymic epithelial stem cell is a medullary thymic epithelial cell and is CD205^{neg}CK5^{pos}KRT14^{pos}.
21. The isolated thymic epithelial stem cell of any one of clauses 1 to 20 wherein the thymic epithelial stem cell is multipotent.
22. The isolated thymic epithelial stem cell of any one of clauses 1 to 21, wherein the isolated thymic epithelial stem cell is a human cell.
23. A method for isolating a thymic epithelial stem cell from thymus, the method comprising:
   (a) obtaining a thymic tissue sample,
   (b) isolating thymic epithelial cells from the thymic tissue sample to obtain a thymic epithelial cell fraction, and
   (c) isolating BCAM^{pos}CD49F^{pos}CD90^{pos}CD24^{neg} thymic epithelial cells from the thymic epithelial cell fraction to obtain isolated thymic epithelial stem cells.
24. The method of clause 23, wherein step (b) comprises the steps of:
   (i) dissociating the thymic tissue sample to obtain single cells;
   (ii) separating the single cells from the thymic tissue sample to obtain a single cell fraction;
   (iii) substantially depleting the single cell fraction of haematopoietic cells and/or red blood cells;
   (iv) isolating EPCAM+ cells to obtain the thymic epithelial cell fraction.
25. The method of clause 23 or 24, wherein step (c) comprises the steps of:
   (i) labelling the thymic epithelial cells for CD49F, CD90, CD24 and BCAM; and
   (ii) separating BCAM^{pos}CD49F^{pos}CD90^{pos}CD24^{neg} cells from the thymic epithelial cell fraction to obtain isolated thymic epithelial stem cells.
26. The method for isolating a thymic epithelial stem cell from thymus according to any of clauses 23-25, comprising the steps of:
   (a) obtaining a thymic tissue sample,
   (b) dissociating the thymic tissue sample to obtain single cells,
   (c) separating the single cells from the thymic tissue sample to obtain a single cell fraction,
   (d) substantially depleting the single cell fraction of haematopoietic cells and/or red blood cells,
   (e) isolating thymic epithelial cells from the single cell fraction,
   (f) labelling the thymic epithelial cells for CD49F, CD90, CD24 and BCAM, and
   (g) isolating BCAM^{pos}CD49F^{pos}CD90^{pos}CD24^{neg} thymic epithelial cells to obtain isolated thymic epithelial stem cells.
27. The method for isolating a thymic epithelial stem cell from thymus according to any one of clauses 24-26, wherein the step of dissociating the thymic tissue sample comprises treating the thymic tissue sample with one or more enzymes.
28. The method for isolating a thymic epithelial stem cell from thymus according to any of clauses 23-27, wherein the step of isolating the thymic epithelial cells comprises isolating EPCAM^{pos} cells.
29. The method for isolating a thymic epithelial stem cell from thymus according to any of clauses 23-28, wherein the step of isolating BCAM^{pos}CD49F^{pos}CD90^{pos}CD24^{neg} epithelial cells is performed using fluorescence-activated cell sorting (FACS).
30. A cell obtainable by the method according to any of clauses 23-29.
31. A method of culturing a thymic epithelial stem cell comprising
   (a) providing at least one isolated thymic epithelial stem cell, and
   (b) culturing the at least one isolated thymic epithelial stem cell under conditions suitable for maintenance and expansion of the at least one isolated thymic epithelial stem cell.
32. The method of clause 31, comprising:
   (a) providing at least one isolated thymic epithelial stem cell,
   (b) contacting the at least one isolated thymic epithelial stem cell with at least one supporting cell, and
   (c) contacting the at least one isolated thymic epithelial stem cell and the at least one supporting cell with a cell culture medium.
33. The method of clause 31 or 32, wherein the at least one supporting cell is a feeder cell.
34. The method of clause 33, wherein the feeder cell is a mouse fibroblast cell.
35. The method of clause 33 or 34, wherein the feeder cell is a sub-lethally irradiated mouse fibroblast cell.
36. The method of any of clauses 31-35, wherein the cell culture medium is cFAD medium.
37. The method of any of clauses 31-36, comprising contacting the at least one isolated thymic epithelial stem cell and/or the at least one feeder cell with a growth promoting agent.
38. The method of culturing a thymic epithelial stem cell according to clause 31, wherein the isolated thymic epithelial stem cell of step (a) is a cortical thymic epithelial stem cell.
39. The method of culturing a thymic epithelial stem cell according to clause 38, wherein the cortical thymic epithelial stem cell is CD205^{pos}EPCAM^{neg}.
40. The method of culturing a thymic epithelial stem cell according to clause 31, wherein the isolated thymic epithelial stem cell of step (a) is a medullary thymic epithelial stem cell.
41. The method of clause 40, wherein the medullary thymic epithelial stem cell is CD205^{neg}EPCAM^{pos}.
42. An isolated population of thymic epithelial stem cells obtainable by the method of any of clauses 23-41.
43. The isolated population of thymic epithelial stem cells of clause 42, wherein the thymic epithelial stem cells display a refractive edges morphology in culture.
44. A method of culturing a cortical thymic epithelial cell derived from a thymic epithelial stem cell, comprising:
   (a) providing a thymic epithelial stem cell; and
   (b) culturing the thymic epithelial stem cell under conditions suitable for obtaining cortical thymic epithelial cells.
45. The method of clause 44, wherein step (b) comprises culturing the thymic epithelial stem cell at an oxygen (O₂) tension of about 1% to about 9%.
46. The method of clause 44 or 45, wherein step (b) comprises culturing the thymic epithelial stem cell at an oxygen (O₂) tension of about 4% to about 6%.
47. A method of culturing a medullary thymic epithelial cell derived from a thymic epithelial stem cell, comprising:
   (a) providing a thymic epithelial stem cell;
   (b) culturing the thymic epithelial stem cell under conditions suitable for obtaining medullary thymic epithelial cells.
48. The method of clause 47, wherein step (b) comprises culturing the thymic epithelial stem cell at an oxygen (O₂) tension of about 10% to about 30%.
49. The method of clause 47 or 48, wherein step (b) comprises culturing the thymic epithelial stem cell at an oxygen (O₂) tension of about 20%.
50. The method of any of clauses 44-49, wherein step (b) comprises culturing the thymic epithelial stem cell at a carbon dioxide (CO₂) tension of about 5% to about 10%.
51. A cortical thymic epithelial cell obtainable by the method of any of clauses 44-46.
52. A medullary thymic epithelial cell obtainable by the method of any of clauses 47-50.
53. A method of differentiating an isolated thymic epithelial stem cell, comprising:
   (a) providing at least one isolated thymic epithelial stem cell,
   (b) contacting the at least one isolated thymic epithelial stem cell to a membrane, wherein the at least one isolated thymic epithelial stem cell is in contact with an upper surface of the membrane,
   (c) providing a cell culture medium, wherein the cell culture medium is positioned below a lower surface of the membrane.
54. The method of clause 53, comprising:
   (a) providing at least one isolated thymic epithelial stem cell,
   (b) contacting the at least one isolated thymic epithelial stem cell to a membrane, wherein the at least one isolated thymic epithelial stem cell is in contact with an upper surface of the membrane,
   (c) contacting the at least one isolated thymic epithelial stem cell and the membrane with a first cell culture medium,
   (d) maintaining the at least one isolated thymic epithelial stem cell under conditions suitable for expansion of the isolated thymic epithelial stem cell to provide an expanded population of thymic epithelial stem cells,
   (d) removing the first cell culture medium,
   (e) providing a second cell culture medium, wherein the second cell culture medium is positioned below a lower surface of the membrane, and
   (f) maintaining the expanded population of thymic epithelial stem cells under conditions suitable for differentiation of the expanded population of thymic epithelial stem cells.
55. A cell culture composition comprising the isolated thymic epithelial stem cell of any one of clauses 1 to 22.
56. A pharmaceutical composition comprising the isolated thymic epithelial stem cell of any one of clauses 1 to 22 and a pharmaceutically acceptable carrier.
57. A thymic construct suitable for implantation into a subject comprising an isolated thymic epithelial stem cell of any one of clauses 1 to 22 or a cell culture composition of clause 55.
58. A method of treating a disease or disorder in a subject, comprising administering to the subject an isolated thymic epithelial stem cell of any one of clauses 1 to 22, a cell culture composition of clause 55, a pharmaceutical composition of clause 56 or a thymic construct of clause 57.
59. The method of clause 58, wherein the disease or disorder is a primary immunodeficiency.
60. The method of clause 58, wherein the disease or disorder is a disease or disorder associated with dysfunctional central tolerance.
61. The method of clause 58, wherein the disease or disorder is autoimmune polyendocrinopathy candidiasis ectodermal dystrophy (APECED).
62. The method of clause 58, wherein the disease or disorder is an autoimmune disease.
63. The method of clause 62, wherein the autoimmune disease is selected from myasthenia gravis, type 1 diabetes, an autoimmune myopathy, and a connective tissue disease.
64. The method of clause 58, wherein the disease or disorder is a cancer.
65. The method of clause 64, wherein the cancer is a thymoma, a thymic carcinoma, a sarcoma, or a neuroendocrine tumour of the thymus.
66. A method of preventing or reversing atrophy of the thymus in a subject, comprising administering to the subject an isolated thymic epithelial stem cell of any one of clauses 1 to 22, a cell culture composition of clause 55, a pharmaceutical composition of clause 56 or a thymic construct of clause 57.
67. A method of treating immunodeficiency in a subject, comprising administering to the subject an isolated thymic epithelial stem cell of any one of clauses 1 to 22, a cell culture composition of clause 55, a pharmaceutical composition of clause 56 or a thymic construct of clause 57.
68. A method of treating athymia in a subject, comprising administering to the subject an isolated thymic epithelial stem cell of any one of clauses 1 to 22, a cell culture composition of clause 55, a pharmaceutical composition of clause 56 or a thymic construct of clause 57.
69. A method of regenerating thymic tissue in a subject, comprising administering to the subject an isolated thymic epithelial stem cell of any one of clauses 1 to 22, a cell culture composition of clause 55, a pharmaceutical composition of clause 56 or a thymic construct of clause 57.
70. A method of increasing, restoring or regenerating a population of circulating T-cells in a subject, comprising administering to the subject an isolated thymic epithelial stem cell of any one of clauses 1 to 22, a cell culture composition of clause 55, a pharmaceutical composition of clause 56 or a thymic construct of clause 57.
71. Use of an isolated thymic epithelial stem cell of any one of clauses 1 to 22 in cell replacement therapy.
72. A method of producing a thymic construct suitable for implantation into a subject, the method comprising the steps of:
   (a) providing an acellular scaffold;
   (b) seeding the acellular scaffold with an isolated thymic epithelial stem cell of any one of clauses 1 to 22; and
   (c) culturing the seeded scaffold to produce said construct.
73. A method of drug screening comprising use of an isolated thymic epithelial stem cell of any one of clauses 1 to 22, a cell culture composition of clause 55, a pharmaceutical composition of clause 56 or a thymic construct of clause 57.
74. A kit for identifying a thymic epithelial stem cell of any one of clauses 1 to 22 comprising a means of identifying one or more cellular markers selected from the list consisting of BCAM, CD49F, CD90 and CD24.
75. The kit of clause 74, wherein the kit comprises binding molecules specific for BCAM, CD49F, CD90 and CD24.
76. An isolated thymic epithelial stem cell, wherein the isolated thymic epithelial stem cell is BCAM^{pos}.
77. The isolated thymic epithelial stem cell of clause 76, wherein the isolated thymic epithelial stem cell expresses at least one cytokeratin gene.
78. The isolated thymic epithelial stem cell of clause 77, wherein the at least one cytokeratin gene is selected from the group consisting of KRT5, KRT8, KRT13, KRT14, KRT15, KRT17, KRT18, and KRT19.
79. The isolated thymic epithelial stem cell of any one of clauses 76 to 78, wherein the isolated thymic epithelial stem cell expresses KRT13, KRT18, and KRT15.
80. The isolated thymic epithelial stem cell of any one of clauses 76 to 79, wherein the isolated thymic epithelial stem cell expresses KRT13, KRT18, and KRT17.
81. The isolated thymic epithelial stem cell of any one of clauses 76 to 80, wherein the isolated thymic epithelial stem cell does not express KRT7, KRT1, KRT10, KRT4, KRT16, KRT23 or KRT6A.
82. The isolated thymic epithelial stem cell of any one of clauses 76 to 81, wherein the isolated thymic epithelial stem cell further expresses at least one selected from the group consisting of: EPCAM, CD49F, FN1, TIMP1, IFITM3, VCAM1, CEPBD, CLU, CCL19, CH25H, COL7A1, CTGF, APOE, FGFR2, BOC, ITGA5, SOX17, LIFR, YAP1, PTGDS, CD34, VWF, SPARC, CAV-1, EPAS-1, TIMP3, COL4A2, COL5A1, COL6A3, TP63 (for example ΔNTP63α) and cMYC.

### REFERENCES

[1] S. Claudinot, M. Nicolas, H. Oshima, A. Rochat, Y. Barrandon, Long-term renewal of hair follicles from clonogenic multipotent stem cells. Proc Natl Acad Sci USA 102, 14677-14682 (2005)
[2] F. M. Watt, Role of integrins in regulating epidermal adhesion, growth and differentiation. EMBO J 21, 3919-3926 (2002)
[3] P. Rama et al., Autologous fibrin-cultured limbal stem cells permanently restore the corneal surface of patients with total limbal stem cell deficiency. Transplantation 72, 1478-1485 (2001)
[4] H. Oshima, A. Rochat, C. Kedzia, K. Kobayashi, Y. Barrandon, Morphogenesis and renewal of hair follicles from adult multipotent stem cells. Cell 104, 233-245 (2001)
[5] A. Rochat, K. Kobayashi, Y. Barrandon, Location of stem cells of human hair follicles by clonal analysis. Cell 76, 1063-1073 (1994)
[6] Y. Barrandon, H. Green, Three clonal types of keratinocyte with different capacities for multiplication. Proc Natl Acad Sci USA 84, 2302-2306 (1987)
[7] T. Sato et al., Single Lgr5 stem cells build crypt-villus structures in vitro without a mesenchymal niche. Nature 459, 262-265 (2009)
[8] T. Sato et al., Paneth cells constitute the niche for Lgr5 stem cells in intestinal crypts. Nature 469, 415-418 (2011)
[9] J. G. Rheinwald, H. Green, Serial cultivation of strains of human epidermal keratinocytes: the formation of keratinizing colonies from single cells. Cell 6, 331-343 (1975)
[10] C. Blanpain, E. Fuchs, Stem cell plasticity. Plasticity of epithelial stem cells in tissue regeneration. Science 344, 1242281 (2014)
[11] P. Bonfanti, Y. Barrandon, G. Cossu, ‘Hearts and bones’: the ups and downs of ‘plasticity’ in stem cell biology. EMBO Mol Med 4, 353-361 (2012)
[12] M. Senoo, F. Pinto, C. P. Crum, F. McKeon, p63 is essential for the proliferative potential of stem cells in stratified epithelia. Cell 129, 523-536 (2007)
[13] E. Di Iorio et al., Isoforms of DeltaNp63 and the migration of ocular limbal cells in human corneal regeneration. Proc Natl Acad Sci USA 102, 9523-9528 (2005)
[14] G. Pellegrini et al., p63 identifies keratinocyte stem cells. Proc Natl Acad Sci USA 98, 3156-3161 (2001))
[15] E. Enzo et al., Single-keratinocyte transcriptomic analyses identify different clonal types and proliferative potential mediated by FOXM1 in human epidermal stem cells. Nat Commun 12, 2505 (2021)
[16] J. Gui, L. M. Mustachio, D. M. Su, R. W. Craig, Thymus Size and Age-related Thymic Involution: Early Programming, Sexual Dimorphism, Progenitors and Stroma. Aging Dis 3, 280-290 (2012)
[17] A. Y. Kreins, P. Bonfanti, E. G. Davies, Current and Future Therapeutic Approaches for Thymic Stromal Cell Defects. Front Immunol 12, 655354 (2021)
[18] D. M. McDonald-McGinn et al., 22q11.2 deletion syndrome. Nat Rev Dis Primers 1, 15071 (2015)
[19] S. W. Rossi, W. E. Jenkinson, G. Anderson, E. J. Jenkinson, Clonal analysis reveals a common progenitor for thymic cortical and medullary epithelium. Nature 441, 988-991 (2006)
[20] C. C. Bleul et al., Formation of a functional thymus initiated by a postnatal epithelial progenitor cell. Nature 441, 992-996 (2006)
[21] K. Wong et al., Multilineage potential and self-renewal define an epithelial progenitor cell population in the adult thymus. Cell Rep 8, 1198-1209 (2014)
[22] A. Ucar et al., Adult thymus contains FoxN1(−) epithelial stem cells that are bipotent for medullary and cortical thymic epithelial lineages. Immunity 41, 257-269 (2014)
[23] M. Sekai, Y. Hamazaki, N. Minato, Medullary thymic epithelial stem cells maintain a functional thymus to ensure lifelong central T cell tolerance. Immunity 41, 753-761 (2014)
[24] Cowan et al. (2020) Postnatal Involution and Counter-Involution of the Thymus, Frontiers in Immunology 11:897
[25] Morales Torres, C., Wu, M. Y., Hobor, S., Wainwright, E. N., Smith, P. D. Swanton, C. Howell, M. Scaffidi, P., Selective inhibition of cancer cell self-renewal through a Quisinostat-histone H1.0 axis. Nature Communications, 2020. 11(1): p. 1792
[26] Bhattacharya, A., Hamilton, A. M., Furberg, H., Pietzak, E., Purdue, M. P., Troester, M. A., Hoadley, K. A., and Love, M. I. (2021). An approach for normalization and quality control for NanoString RNA expression data. Brief Bioinform 22
[27] J. L. Bautista et al., Single-cell transcriptional profiling of human thymic stroma uncovers novel cellular heterogeneity in the thymic medulla. Nat Commun 12, 1096 (2021)
[28] J. E. Park et al., A cell atlas of human thymic development defines T cell repertoire formation. Science 367, (2020)
[29] J. E. Cowan et al., Myc controls a distinct transcriptional program in fetal thymic epithelial cells that determines thymus growth. Nat Commun 10, 5498 (2019)
[30] J. R. Goldsmith et al., TNFAIP8 controls murine intestinal stem cell homeostasis and regeneration by regulating microbiome-induced Akt signaling. Nat Commun 11, 2591 (2020);
[31] B. Shapiro, P. Tocci, G. Haase, N. Gavert, A. Ben-Ze'ev, Clusterin, a gene enriched in intestinal stem cells, is required for L1-mediated colon cancer metastasis. Oncotarget 6, 34389-34401 (2015);
[32] V. Barbaro et al., C/EBPdelta regulates cell cycle and self-renewal of human limbal stem cells. J Cell Biol 177, 1037-1049 (2007)
[33] M. Salery, A. Godino, E. J. Nestler, Drug-activated cells: From immediate early genes to neuronal ensembles in addiction. Adv Pharmacol 90, 173-216 (2021)
[34] J. Santos et al., The stromal gene encoding the CD274 antigen as a genetic modifier controlling survival of mice with gamma-radiation-induced T-cell lymphoblastic lymphomas. Oncogene 29, 5265-5273 (2010)
[35] A. M. Marchevsky, A. E. Walts, PD-L1, PD-1, CD4, and CD8 expression in neoplastic and nonneoplastic thymus. Hum Pathol 60, 16-23 (2017)
[36] S. K. Padda et al., Diffuse high intensity PD-L1 staining in thymic epithelial tumors. J Thorac Oncol 10, 500-508 (2015)
[37] L. W. Plasschaert et al., A single-cell atlas of the airway epithelium reveals the CFTRrich pulmonary ionocyte. Nature 560, 377-381 (2018)
[38] Park, N. I., Guilhamon, P., Desai, K., McAdam, R. F., Langille, E., O'Connor, M., Lan, X., Whetstone, H., Coutinho, F. J., Vanner, R. J., et al. (2017) ASCL1 Reorganizes Chromatin to Direct Neuronal Fate and Suppress Tumorigenicity of Glioblastoma Stem Cells. Cell Stem Cell 21, 411
[39] Wapinski, O. L., Vierbuchen, T., Qu, K., Lee, Q. Y., Chanda, S., Fuentes, D. R., Giresi, P. G., Ng, Y. H., Marro, S., Neff, N. F., et al. (2013) Hierarchical mechanisms for direct reprogramming of fibroblasts to neurons. Cell 155, 621-635
[40] Campbell, K. R., and Yau, C. (2019). A descriptive marker gene approach to single-cell pseudotime inference. Bioinformatics 35, 28-35.
[41] K. E. Boonekamp et al., Long-term expansion and differentiation of adult murine epidermal stem cells in 3D organoid cultures. Proc Natl Acad Sci USA 116, 14630-14638 (2019)
[42] M. Ohyama et al., Characterization and isolation of stem cell-enriched human hair follicle bulge cells. J Clin Invest 116, 249-260 (2006)
[43] R. K. Ward, P. N. Nation, M. Maxwell, C. L. Barker, R. H. Clothier, Evaluation in vitro of epidermal cell keratinization. Toxicol In Vitro 11, 633-636 (1997)
[44] Campinoti, S., Gjinovci, A., Ragazzini, R. et al. Reconstitution of a functional human thymus by postnatal stromal progenitor cells and natural whole-organ scaffolds. Nat Commun 11, 6372 (2020)
[45] Donati, G., and Watt, F. M. (2015). Stem cell heterogeneity and plasticity in epithelia. Cell Stem Cell 16, 465-476
[46] A. Bartolini et al., BCAM and LAMA5 Mediate the Recognition between Tumor Cells and the Endothelium in the Metastatic Spreading of KRAS-Mutant Colorectal Cancer. Clin Cancer Res 22, 4923-4933 (2016)
[47] M. Udani et al., Basal cell adhesion molecule/lutheran protein. The receptor critical for sickle cell adhesion to laminin. J Clin Invest 101, 2550-2558 (1998)
[48] Y. Hamazaki et al., Medullary thymic epithelial cells expressing Aire represent a unique lineage derived from cells expressing claudin. Nat Immunol 8, 304-311 (2007)
[49] N. Girard, Thymic epithelial tumours: from basic principles to individualised treatment strategies. Eur Respir Rev 22, 75-87 (2013)
[50] R. Moll, M. Divo, L. Langbein, The human keratins: biology and pathology. Histochem Cell Biol 129, 705-733 (2008)
[51] K. H. Kim, J. G. Rheinwald, E. V. Fuchs, Tissue specificity of epithelial keratins: differential expression of mRNAs from two multigene families. Mol Cell Biol 3, 495-502 (1983)
[52] P. Bonfanti, Y. Barrandon, G. Cossu (2012); P. Bonfanti et al., Microenvironmental reprogramming of thymic epithelial cells to skin multipotent stem cells. Nature 466, 978-982 (2010)
[53] X. Wu et al., Intrinsic Immunity Shapes Viral Resistance of Stem Cells. Cell 172, 423-438 e425 (2018)
[54] U. S. Rajapaksa, C. Jin, T. Dong, Malignancy and IFITM3: Friend or Foe? Front Oncol 10, 593245 (2020)
[55] M. Abdulhasan et al., Stress Decreases Host Viral Resistance and Increases Covid Susceptibility in Embryonic Stem Cells. Stem Cell Rev Rep, (2021)
[56] A. D. Kenney et al., IFITM3 protects the heart during influenza virus infection. Proc Natl Acad Sci USA 116, 18607-18612 (2019)
[57] A. D. Colantonio et al., IFN-alpha is constitutively expressed in the human thymus, but not in peripheral lymphoid organs. PLoS One 6, e24252 (2011)
[58] J. C. Adams, F. M. Watt, Fibronectin inhibits the terminal differentiation of human keratinocytes. Nature 340, 307-309 (1989)
[59] T. Hirsch et al., Regeneration of the entire human epidermis using transgenic stem cells. Nature 551, 327-332 (2017)
[60] P. Rama et al., Limbal stem-cell therapy and long-term corneal regeneration. N Engl J Med 363, 147-155 (2010)

1. An isolated thymic epithelial stem cell, wherein the isolated thymic epithelial stem cell is BCAM^{pos}, CD49F^{pos}, CD90^{pos} and CD24^{neg}.

2. The isolated thymic epithelial stem cell of claim 1, wherein the isolated thymic epithelial stem cell expresses at least one cytokeratin gene.

3. The isolated thymic epithelial stem cell of claim 1 or claim 2, wherein the at least one cytokeratin gene is selected from the group consisting of KRT5, KRT8, KRT13, KRT14, KRT15, KRT17, KRT18, and KRT19, or combinations thereof.

4. The isolated thymic epithelial stem cell of any one of claims 1 to 4, wherein the isolated thymic epithelial stem cell further expresses at least one selected from the group consisting of: EPCAM, CD49F, FN1, TIMP1, IFITM3, VCAM1, CEPBD, CLU, CCL19, CH25H, COL7A1, CTGF, APOE, FGFR2, BOC, ITGA5, SOX17, LIFR, YAP1, PTGDS, CD34, VWF, SPARC, CAV-1, EPAS-1, TIMP3, COL4A2, COL5A1, COL6A3, TP63 (for example ΔNTP63α) and cMYC, or combinations thereof.

5. The isolated thymic epithelial stem cell of any one of claims 1 to 5, wherein the isolated thymic epithelial stem cell expresses at least one selected from the group consisting of CCNA2, AURKB, FOXM1, ANLN, LMNB1, HMGB2, or combinations thereof.

6. An isolated thymic epithelial stem cell, wherein the isolated thymic epithelial stem cell is capable of ex vivo self-renewal.

7. An isolated thymic epithelial stem cell, wherein the isolated thymic epithelial stem cell exhibits long-term expansion capacity in vitro.

8. The isolated thymic epithelial stem cell of claim 7, wherein the isolated thymic epithelial stem cell is capable of at least 15 population doublings in vitro.

9. An isolated thymic epithelial stem cell, wherein the isolated thymic epithelial stem cell is capable of differentiating into cortical thymic epithelial cells and/or medullary epithelial cells.

10. The isolated thymic epithelial stem cell of any preceding claim, wherein the thymic epithelial stem cell is a cortical thymic epithelial cell and is CD205^{pos}KRT5^{neg}KRT14^{neg}.

11. The isolated thymic epithelial stem cell of any preceding claim, wherein the thymic epithelial stem cell is a medullary thymic epithelial cells and is CD205^{neg}KRT5^{pos} and/or KRT14^{pos}.

12. The isolated thymic epithelial stem cell of any one of claims 1 to 11 wherein the thymic epithelial stem cell is multipotent.

13. The isolated thymic epithelial stem cell of any one of claims 1 to 12, wherein the isolated thymic epithelial stem cell is a human cell.

14. A method for isolating a thymic epithelial stem cell from thymus, the method comprising:
(a) obtaining a thymic tissue sample,
(b) isolating thymic epithelial cells from the thymic tissue sample to obtain a thymic epithelial cell fraction, and
(c) isolating BCAM^{pos}CD49F^{pos}CD90^{pos}CD24^{neg} thymic epithelial cells from the thymic epithelial cell fraction to obtain isolated thymic epithelial stem cells.

15. The method for isolating a thymic epithelial stem cell from thymus according to claim 14, wherein the step of isolating BCAM^{pos}CD49F^{pos}CD90^{pos}CD24^{neg} epithelial cells is performed using fluorescence-activated cell sorting (FACS).

16. A method of culturing a thymic epithelial stem cell comprising:
(a) providing at least one isolated thymic epithelial stem cell, and
(b) culturing the at least one isolated thymic epithelial stem cell under conditions suitable for maintenance and expansion of the at least one isolated thymic epithelial stem cell.

17. The method of culturing a thymic epithelial stem cell according to claim 16, wherein the isolated thymic epithelial stem cell of step (a) is a cortical thymic epithelial stem cell.

18. The method of culturing a thymic epithelial stem cell according to claim 16, wherein the isolated thymic epithelial stem cell of step (a) is a medullary thymic epithelial stem cell.

19. A method of culturing a cortical thymic epithelial cell derived from a thymic epithelial stem cell, comprising:
(a) providing a thymic epithelial stem cell, and
(b) culturing the thymic epithelial stem cell under conditions suitable for obtaining cortical thymic epithelial cells.

20. The method of claim 19, wherein step (b) comprises culturing the thymic epithelial stem cell at an oxygen (O₂) tension of about 1% to about 9%.

21. The method of claim 19 or 20, wherein step (b) comprises culturing the thymic epithelial stem cell at an oxygen (O₂) tension of about 4% to about 6%.

22. A method of differentiating an isolated thymic epithelial stem cell, comprising:
(a) providing at least one isolated thymic epithelial stem cell,
(b) contacting the at least one isolated thymic epithelial stem cell to a membrane, wherein the at least one isolated thymic epithelial stem cell is in contact with an upper surface of the membrane,
(c) providing a cell culture medium, wherein the cell culture medium is positioned below a lower surface of the membrane.

23. A thymic construct suitable for implantation into a subject comprising an isolated thymic epithelial stem cell of any one of claims 1 to 13.

24. A method of treating a disease or disorder in a subject, comprising administering to the subject an isolated thymic epithelial stem cell of any one of claims 1 to 13, or a thymic construct of claim 23.

25. A method of producing a thymic construct suitable for implantation into a subject, the method comprising the steps of:
(a) providing an acellular scaffold;
(b) seeding the acellular scaffold with an isolated thymic epithelial stem cell of any one of claims 1 to 13; and
(c) culturing the seeded scaffold to produce said construct.
